SEQUENCE LISTING (14 sequences)

SEQ ID NO: 1 - DNA, 18 bp, Mus musculus
agtggcaaagtggagatt

SEQ ID NO: 2 - DNA, 19 bp, Mus musculus
gtggagtcatactggaaca

SEQ ID NO: 3 - DNA, 21 bp, Mus musculus
tgtccctaatgacagctcctt

SEQ ID NO: 4 - DNA, 21 bp, Mus musculus
gcatccacccaaatgacacat

SEQ ID NO: 5 - DNA, 22 bp, Mus musculus
tgcggattgagtaccaagatgg

SEQ ID NO: 6 - DNA, 21 bp, Mus musculus
ctgtccgtttatccttgcaca

SEQ ID NO: 7 - DNA, 23 bp, Mus musculus
taccatgaggtcacttcagatgc

SEQ ID NO: 8 - DNA, 20 bp, Mus musculus
gcactctcggcctacattgg

SEQ ID NO: 9 - DNA, 21 bp, Mus musculus
cccattcctcgtcacgatctc

SEQ ID NO: 10 - DNA, 23 bp, Mus musculus
tcagactggtttgggataggttt

SEQ ID NO: 11 - DNA, 27 bp, Mus musculus
gatatgaagccatgtactccttactgg

SEQ ID NO: 12 - DNA, 20 bp, Mus musculus
ggcagaggtgcagtctgcat

SEQ ID NO: 13 - DNA, 21 bp, Mus musculus
ggaagaccactcgcattcctt

SEQ ID NO: 14 - DNA, 22 bp, Mus musculus
gtaatcagcaaccattgggtca

(19) United States
(12) Patent Application Publication
(10) Pub. No.: US 2022/0233743 A1
(43) Pub. Date: Jul. 28, 2022

(54) WOUND CLOSURE ENHANCEMENT METHODS AND MATERIALS VIA MANIPULATION OR AUGMENTATION OF LIPIN-1

(71) Applicant: Board of Supervisors of Louisiana State University and Agricultural and Mechanical College, Baton Rouge, LA (US)

(72) Inventors: Matthew WOOLARD, Shreveport, LA (US); Robert SCHILKE, Shreveport, LA (US); Cassidy BLACKBURN, Shreveport (US)

(73) Assignee: Board of Supervisors of Louisiana State University and Agricultural and Mechanical College, Baton Rouge, LA (US)

(21) Appl. No.: 17/121,685

(22) Filed: Dec. 14, 2020

Related U.S. Application Data
(60) Provisional application No. 62/947,523, filed on Dec. 12, 2019.

(51) Int. Cl. A61L 15/44; A61P 17/02; A61L 15/28; A61F 13/00

(52) U.S. Cl. CPC A61L 15/44; A61P 17/02; A61L 15/28; A61K 38/1709; A61F 13/00042; A61L 2300/404; A61L 2400/04; A61F 13/00063

(57) ABSTRACT

A composition for wound healing, comprising an amount of a macrophage proresolving polarizer, wherein the amount is effective to promote wound healing. The composition includes wherein the macrophage proresolving polarizer is lipin-1. The composition includes wherein the composition includes one, two, or three of IL-4, apoptotic cells (ACs), and AC derived lipids. The composition includes wherein the macrophage proresolving polarizer is a lipin-1 transcriptional coregulatory activity promoter. The composition includes, wherein the lipin-1 transcriptional coregulatory activity promoter is an inhibitor of lipin-1 macrophage pro-inflammatory responses enzymatic activity. A method for promoting wound healing, comprising contacting a wound on a skin of a mammal with the composition. A kit comprising one or more containers including the composition in sterile packaging. A wound healing device comprising a substrate and an amount of an amount of a macrophage proresolving polarizer, wherein the amount is effective to promote wound healing.

### CROSS REFERENCE TO RELATED APPLICATIONS/PRIORITY

The present invention claims priority to United States Provisional Patent Application No. 62/947,523 filed Dec. 12, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. RO1 HL131844 (MW) and RO1 HL119225 (BF) awarded by the National Heart, Lung, and Blood Institute. The government has certain rights in the invention.

### BACKGROUND

Open wounds affect both those in critical and chronic conditions. Trauma, including bleeding wounds, is the number one cause of death for Americans between the ages of 1 and 46 and cost over $600 billion a year in health care costs and lost productivity. Chronic wounds, or wounds that are slow to heal currently affect 6.5 million people in the U.S. and the numbers will likely increase, according to the U.S. National Institutes of Health. If untreated, chronic wounds can lead to loss of limbs or even death. With the ageing population and increase in incidence of diabetes, this is expected to impact a greater number of people each year. According to the WHO, open wounds have a potential for serious bacterial wound infections, including gas gangrene and tetanus, and these in turn may lead to long term disabilities, chronic wound or bone infection, and death. Wound infection is particularly of concern when injured patients present late for definitive care, or in disasters where large numbers of injured survivors exceed available trauma care capacity. For the foregoing reasons, there is a pressing, but seemingly irresolvable need for a method, substances and devices to expedite wound closure and healing.

### SUMMARY

Wherefore, it is an object of the present invention to overcome the above-mentioned shortcomings and drawbacks associated with the current technology.

The presently claimed invention relates to compositions for wound healing, comprising an amount of a macrophage proresolving polarizer, wherein the amount is effective to promote wound healing. According to a further embodiment, the macrophage proresolving polarizer is lipin-1. According to a further embodiment, the composition includes one, two, or three of IL-4, apoptotic cells (ACs), and AC derived lipids. According to a further embodiment, the macrophage proresolving polarizer is a lipin-1 transcriptional coregulatory activity promoter. According to a further embodiment, the lipin-1 transcriptional coregulatory activity promoter is an inhibitor of lipin-1 macrophage pro-inflammatory responses enzymatic activity. According to a further embodiment, the lipin-1 macrophage pro-inflammatory response enzymatic activity inhibitor is an antibody. According to a further embodiment, the composition is in one of powder form, aerosol form, and gel form. According to a further embodiment, the composition is applied to a support. According to a further embodiment, the composition comprises an adsorbent. According to a further embodiment, the composition comprises a blood clotting agent. According to a further embodiment, the composition comprises a blood vessel constricting agent. According to a further embodiment, the composition comprises a local anesthetic agent. According to a further embodiment, the composition comprises an antimicrobial agent.

The presently claimed invention further relates to methods for promoting wound healing, comprising contacting a wound on a skin of a mammal with the composition.

The presently claimed invention relates to kits comprising one or more containers including the composition in sterile packaging.

The presently claimed invention relates to a wound healing devices comprising a substrate and an amount of an amount of a macrophage proresolving polarizer, wherein the amount is effective to promote wound healing. According to a further embodiment, the wound healing device comprises one, two, or three of a blood vessel constricting agent, an adsorbent, and a blood clotting agent. According to a further embodiment, the macrophage proresolving polarizer is lipin-1. According to a further embodiment, the composition includes one, two, three, four, or five of IL-4, apoptotic cells (ACs), AC derived lipids, Ly6C^{hi}, and Ly6C^{lo}. According to a further embodiment, the macrophage proresolving polarizer is a lipin-1 transcriptional coregulatory activity promoter.

Macrophage responses contribute to a diverse array of pathologies ranging from infectious disease to sterile inflammation. Polarization of macrophages determines their cellular function within biological processes. Lipin-1 is a phosphatidic acid phosphatase in which its enzymatic activity contributes to macrophage pro-inflammatory responses. Lipin-1 also possesses transcriptional co-regulator activity and whether this activity is required for macrophage polarization is unknown. Using mice that lack only lipin-1 enzymatic activity or both enzymatic and transcriptional coregulator activities from myeloid cells, the inventors investigated the contribution of lipin-1 transcriptional co-regulator function toward macrophage wound healing polarization. Macrophages lacking both lipin-1 activities did not elicit IL-4 mediated gene expression to levels seen in either wild-type or lipin-1 enzymatically deficient macrophages. Furthermore, mice lacking myeloid-associated lipin-1 have impaired full thickness excisional wound healing compared to wild-type mice or mice only lacking lipin-1 enzymatic activity from myeloid cell. The inventors' study provides evidence that lipin-1 transcriptional co-regulatory activity contributes to macrophage polarization and influences wound healing in vivo.

Macrophages reprogram their metabolism to promote appropriate responses. Proresolving macrophages primarily use fatty acid oxidation as an energy source. Metabolites generated during the catabolism of fatty acids aid in the resolution of inflammation and tissue repair, but the regulatory mechanisms that control lipid metabolism in macrophages are not fully elucidated. Lipin-1, a phosphatidic acid phosphatase that has transcriptional coregulator activity, regulates lipid metabolism in a variety of cells. In this current study, the inventors show that lipin-1 is required for increased oxidative phosphorylation in IL-4 stimulated mouse (Mus musculus) macrophages. The inventors also show that the transcriptional coregulatory function of lipin-1 is required for β-oxidation in response to palmitate (free fatty acid) and (human) apoptotic cell (AC) stimulation. Mouse bone marrow-derived macrophages lacking lipin-1 have a reduction in critical TCA cycle metabolites following IL-4 stimulation, suggesting a break in the TCA cycle that is supportive of lipid synthesis rather than lipid catabolism. Together, the inventors' data demonstrate that lipin-1 regulates cellular metabolism in macrophages in response to proresolving stimuli and highlights the importance of aligning macrophage metabolism with macrophage phenotype.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### INCORPORATION OF SEQUENCE LISTING (TEXT FILE)

This application contains a text file named LSUHS_P106_AUS_ST25.txt, which is 2,431 bytes (measured in MS-DOS), which was created on Nov. 22, 2021, and is hereby incorporated by reference into the specification of this application in its entirety. The text file sequence listing contains the PCR primer sequence listings that are listed in FIG. 42 of the Drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1-3 show lipin-1 enzymatic activity does not contribute to oxidative phosphorylation. BMDMs isolated from WT and EKO mice were treated with or without 40 ng/ml IL-4 for 4 h. Oxygen consumption was analyzed via Seahorse extracellular flux analyzer.

FIG. 4 shows untreated WT and EKO macrophages.

FIG. 5 shows WT and EKO BMDMs treated with IL-4.

FIG. 6 shows basal and maximal (FCCP) OCR of WT and EKO BMDMs treated with and without IL-4. Graphed data represents mean OCR with SEM. n=3.

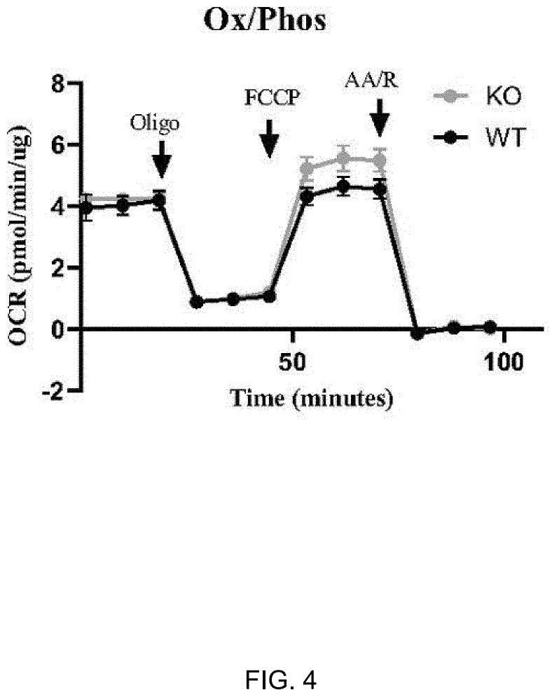

FIGS. 4-6 show lipin-1 transcriptional coregulator function contributes to oxidative phosphorylation. BMDMs isolated from WT and KO mice were treated with or without 40 ng/ml IL-4 for 4 h. Oxygen consumption was analyzed via Seahorse extracellular flux analyzer.

FIG. 4 shows untreated WT and KO macrophages.

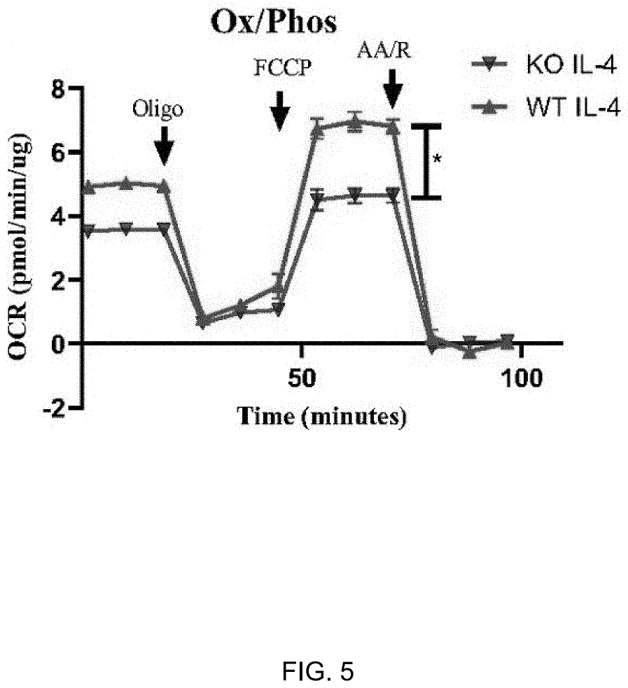

FIG. 5 shows WT and EKO BMDMs treated with IL-4.

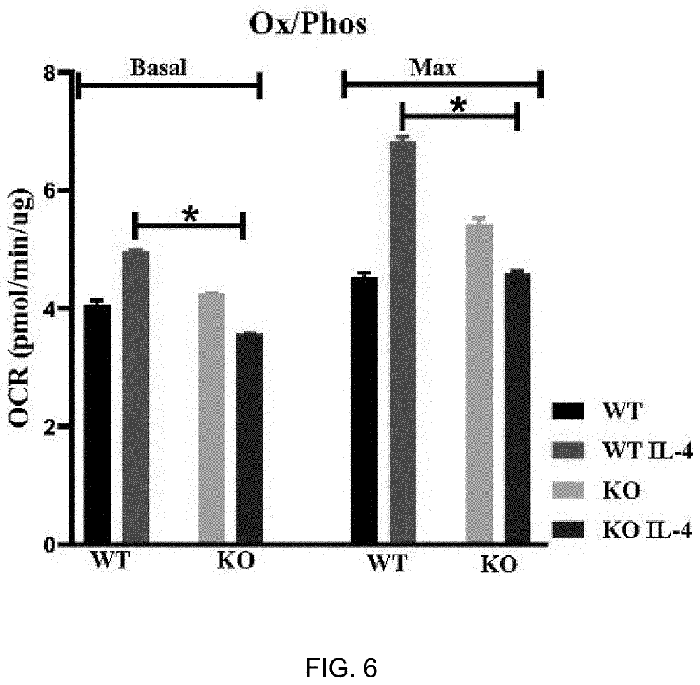

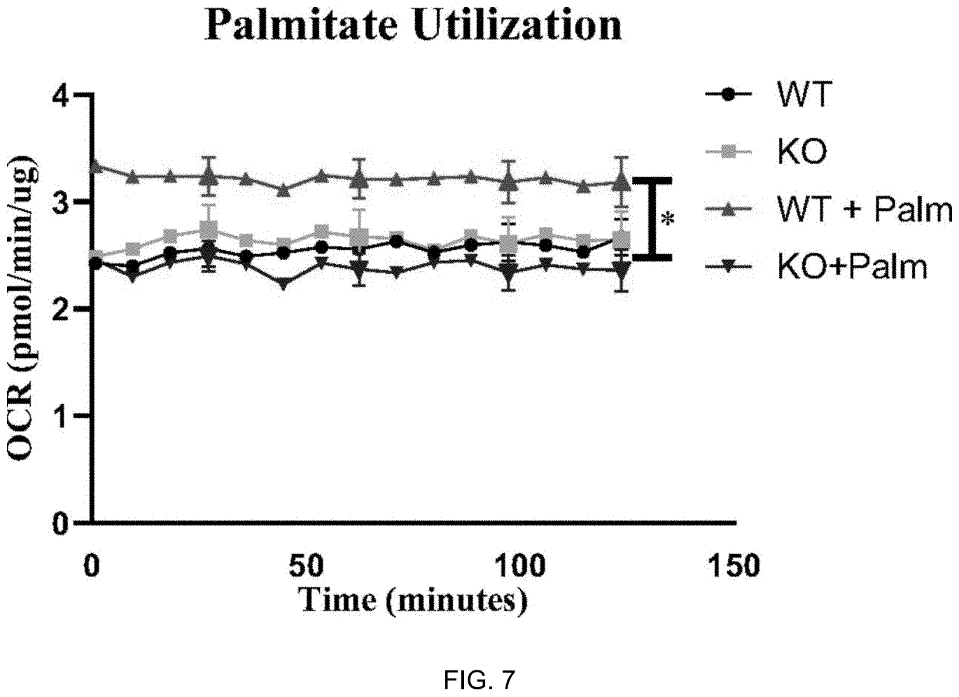

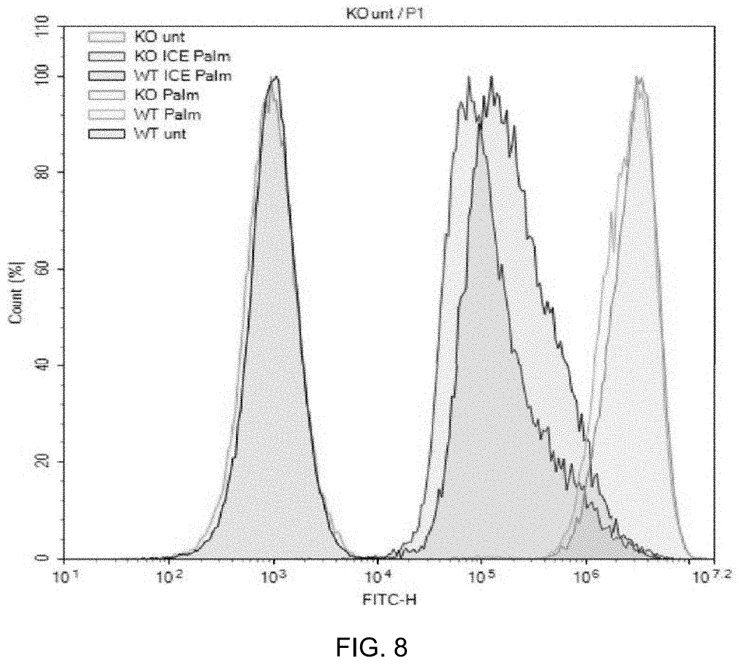

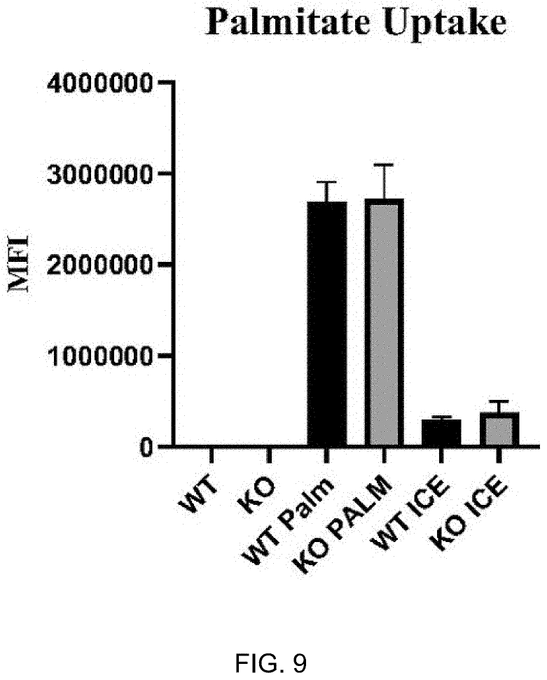

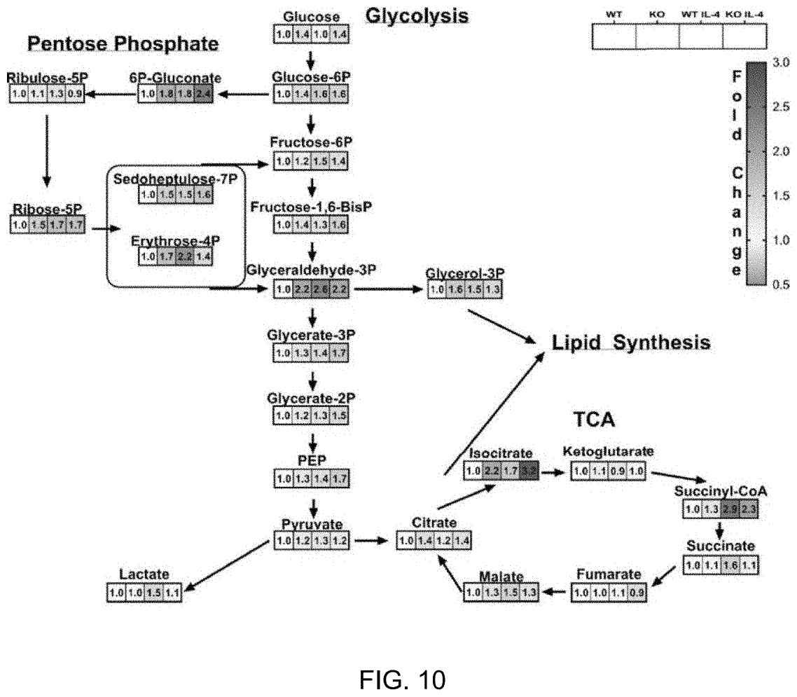

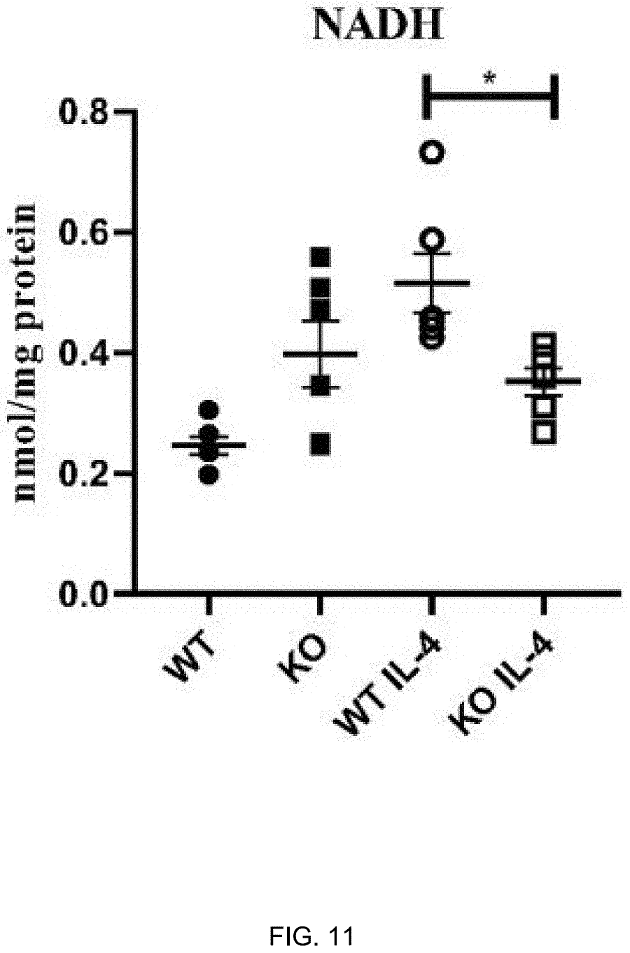

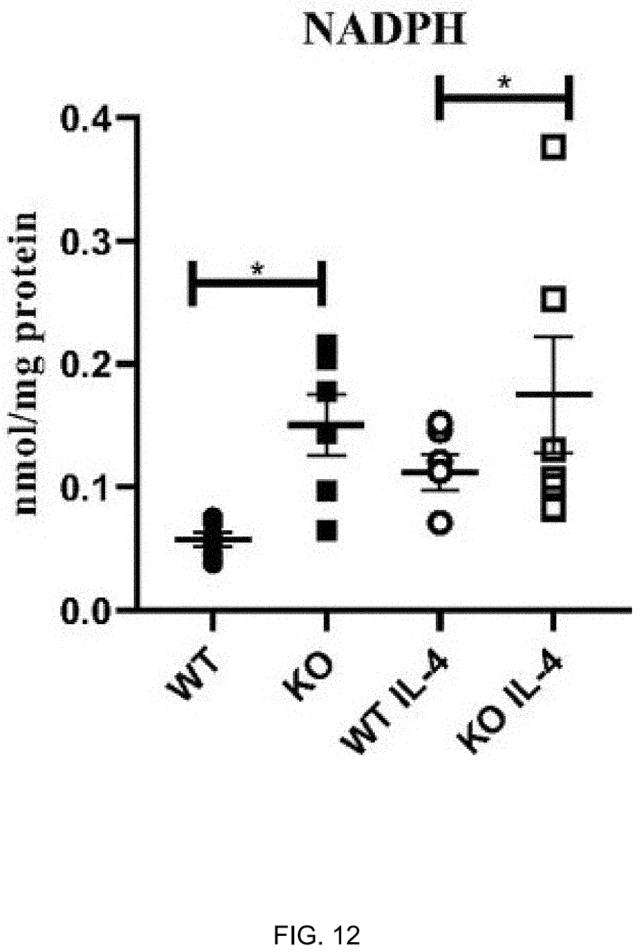

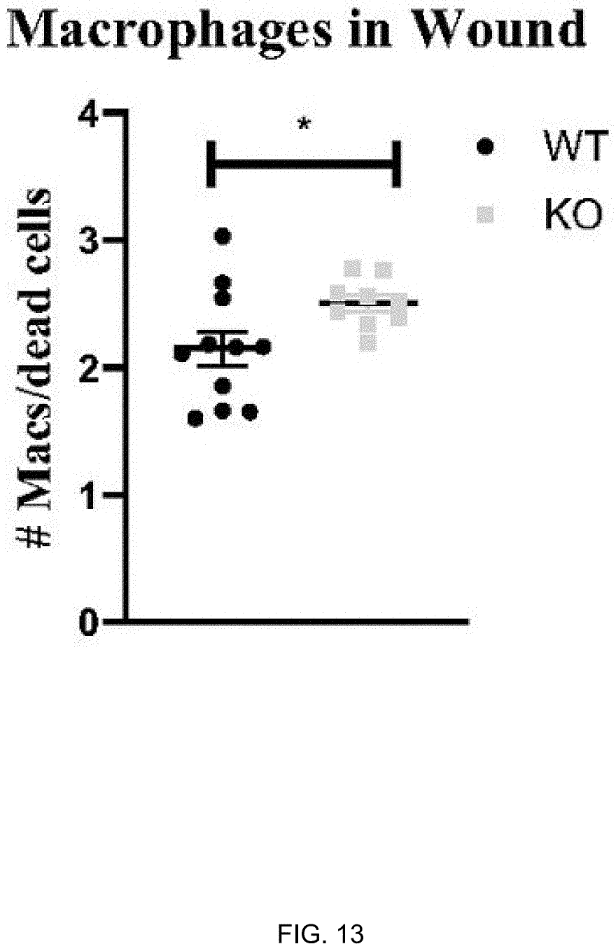

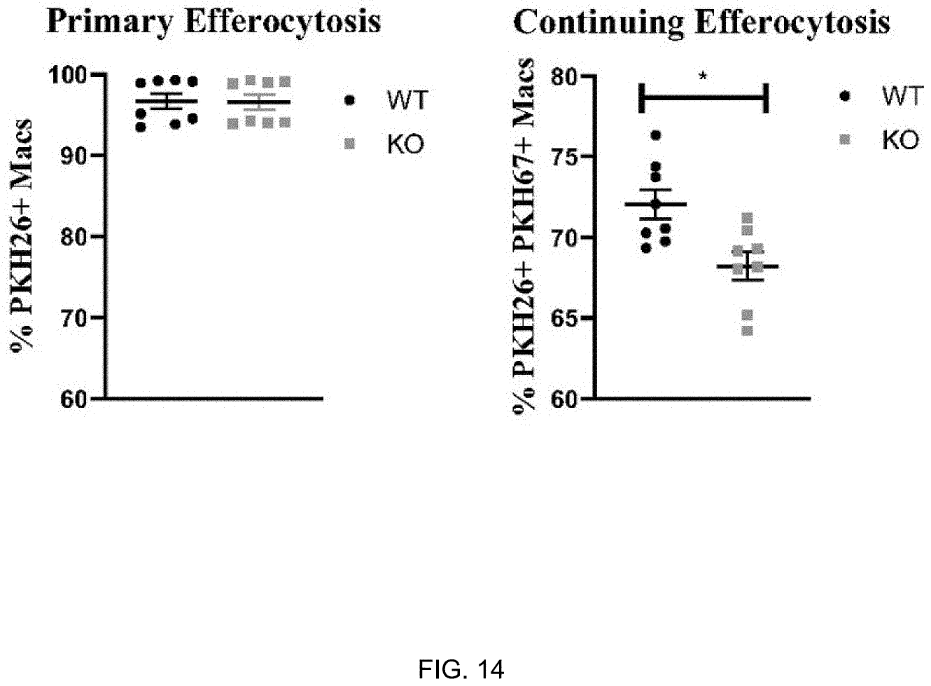

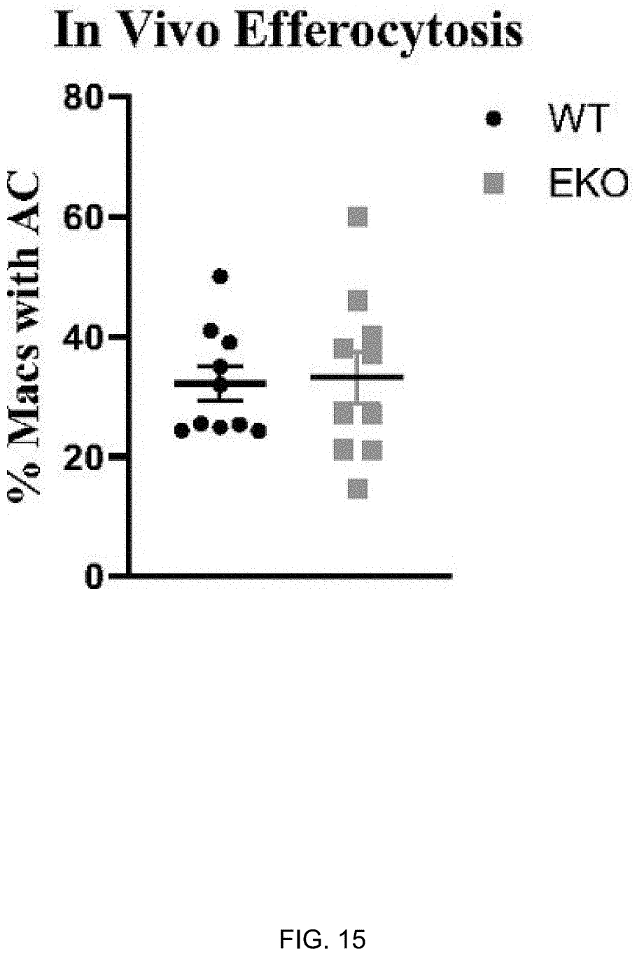

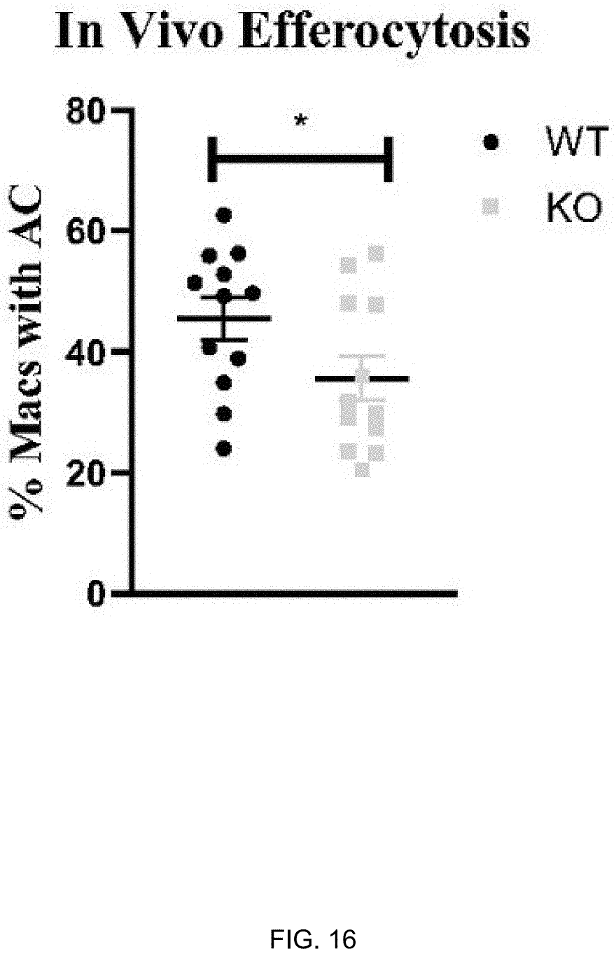

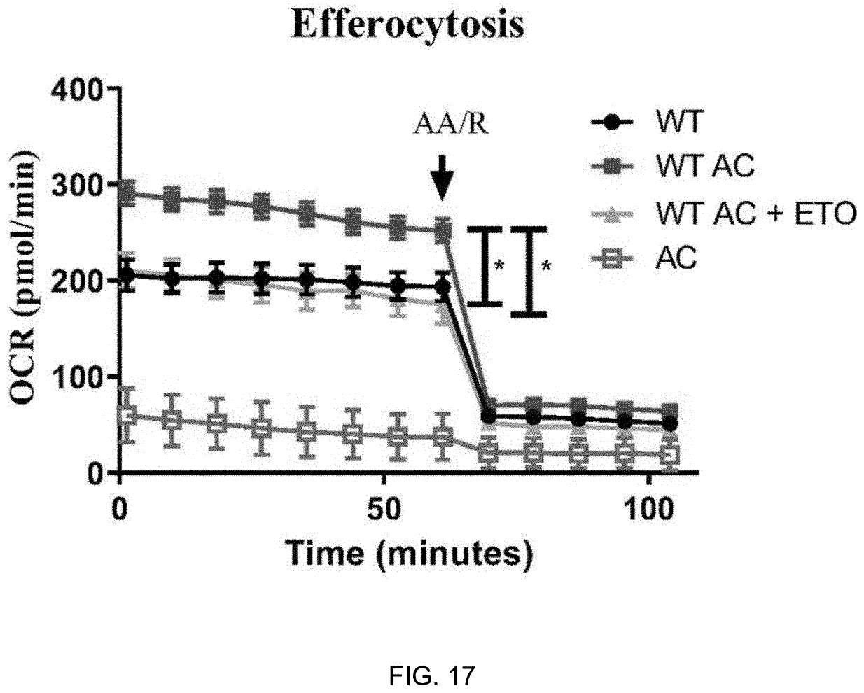

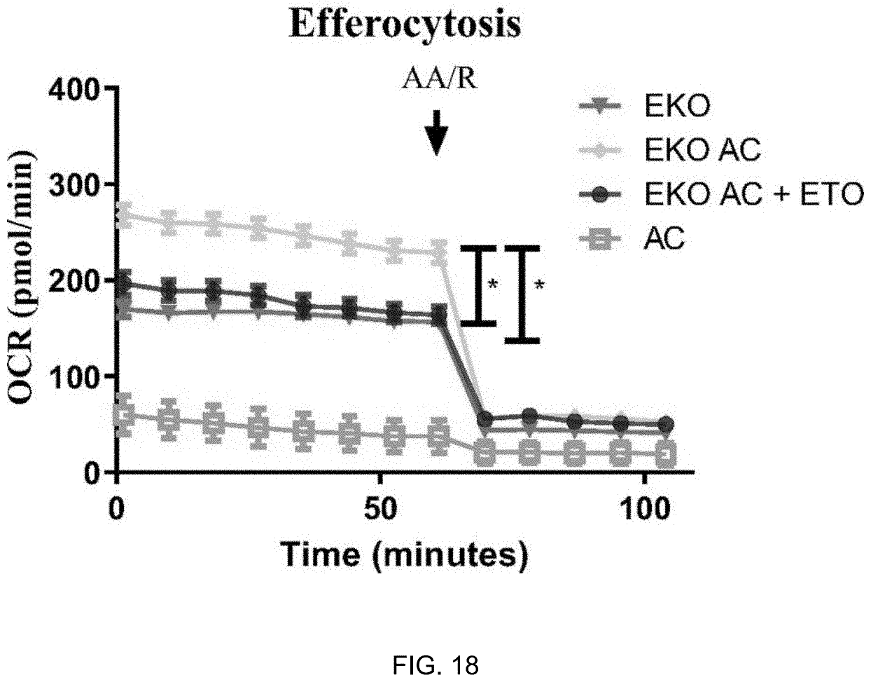

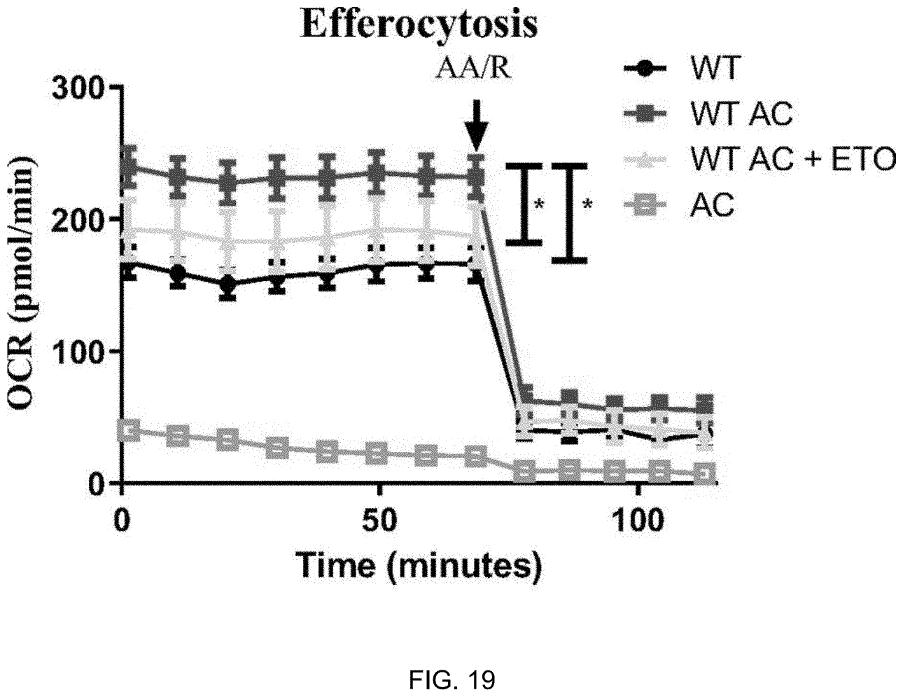

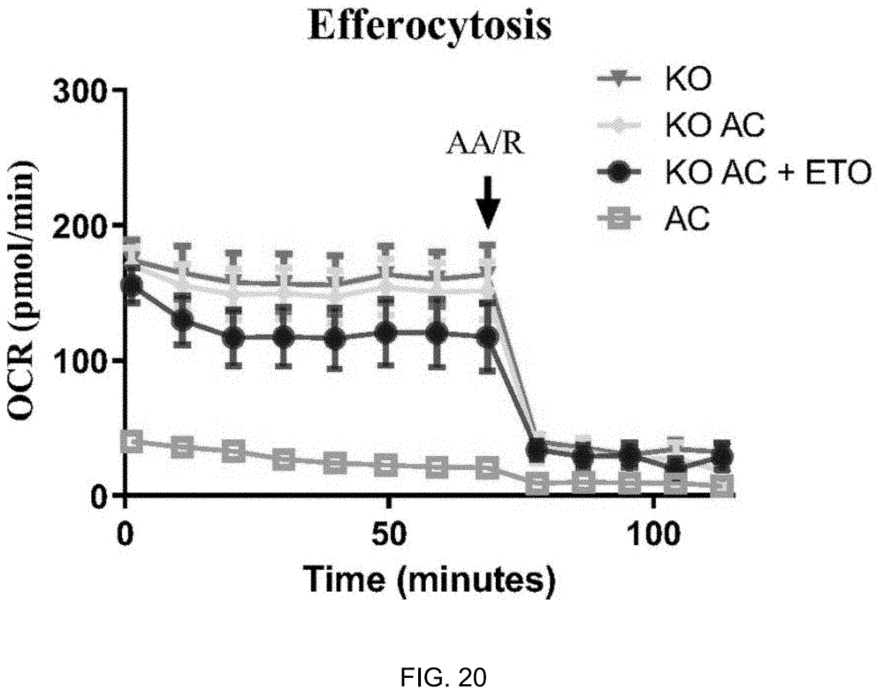

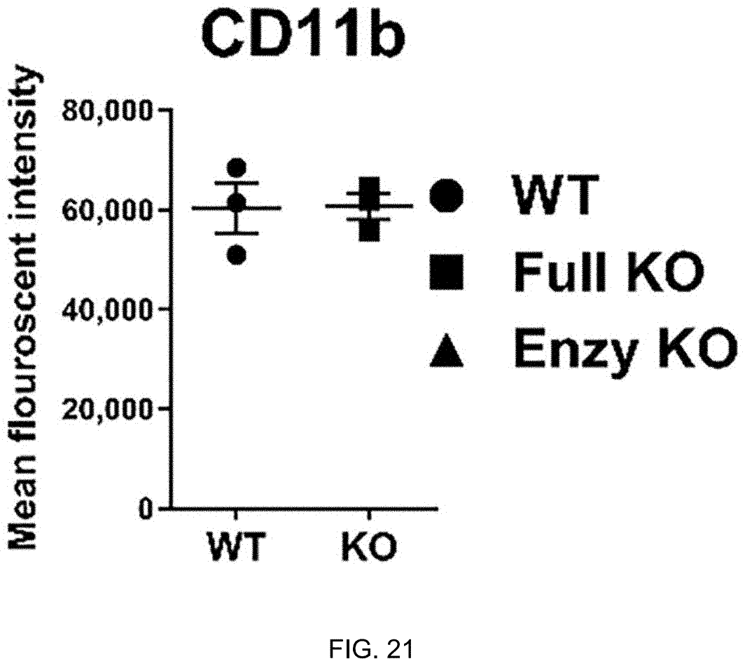

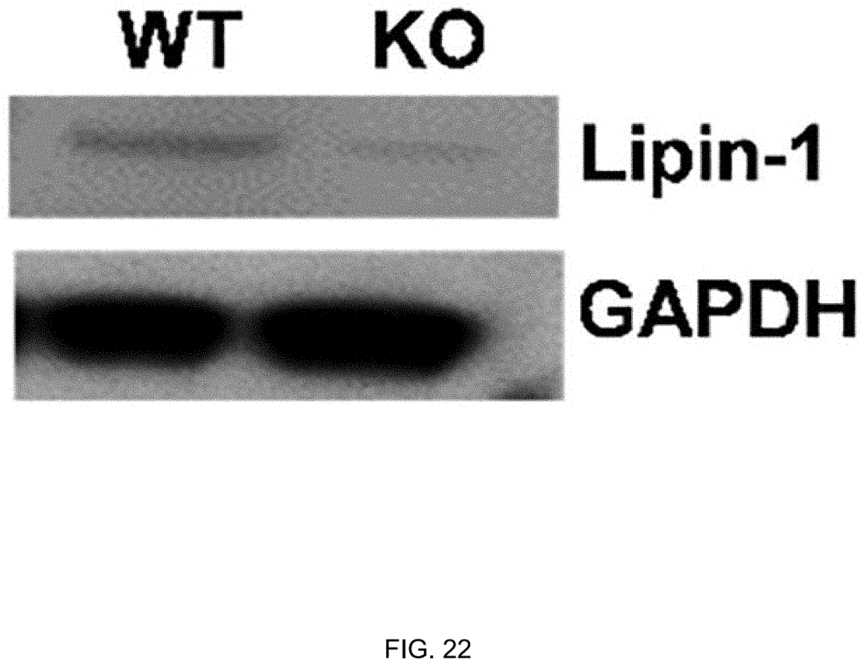

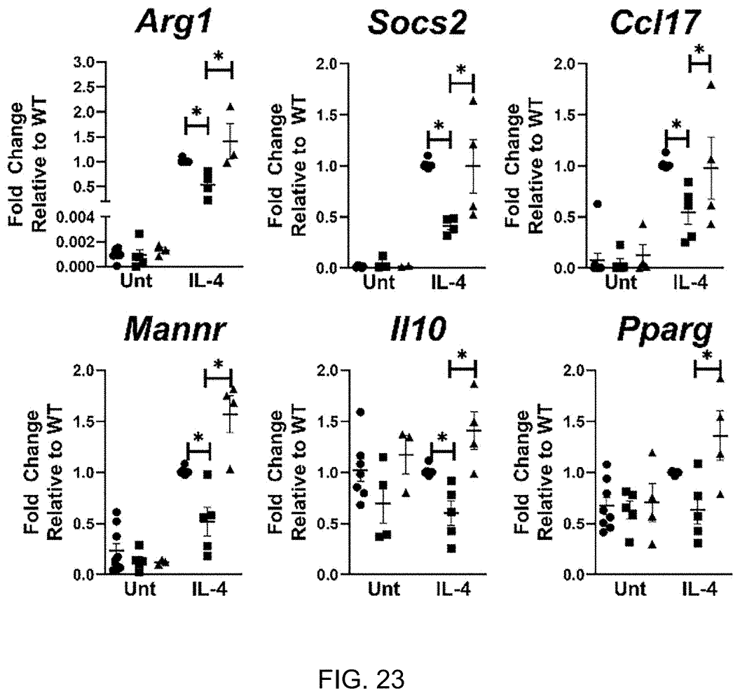

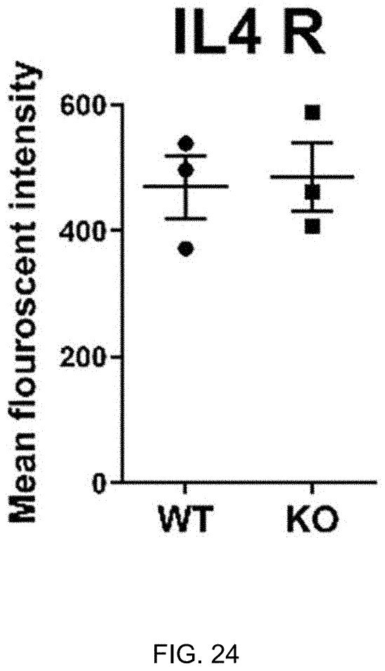

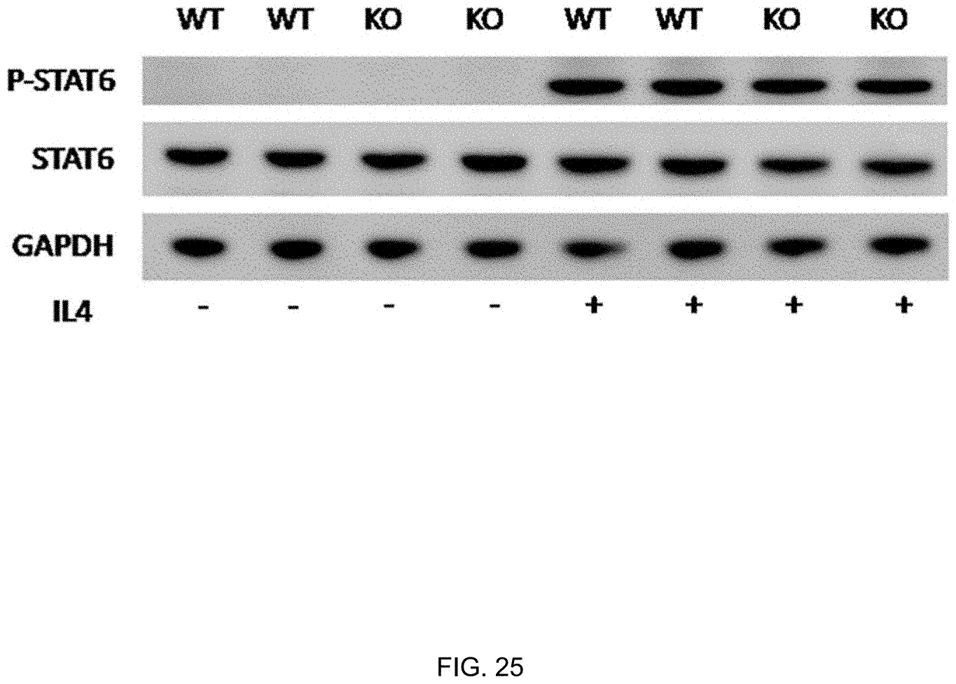

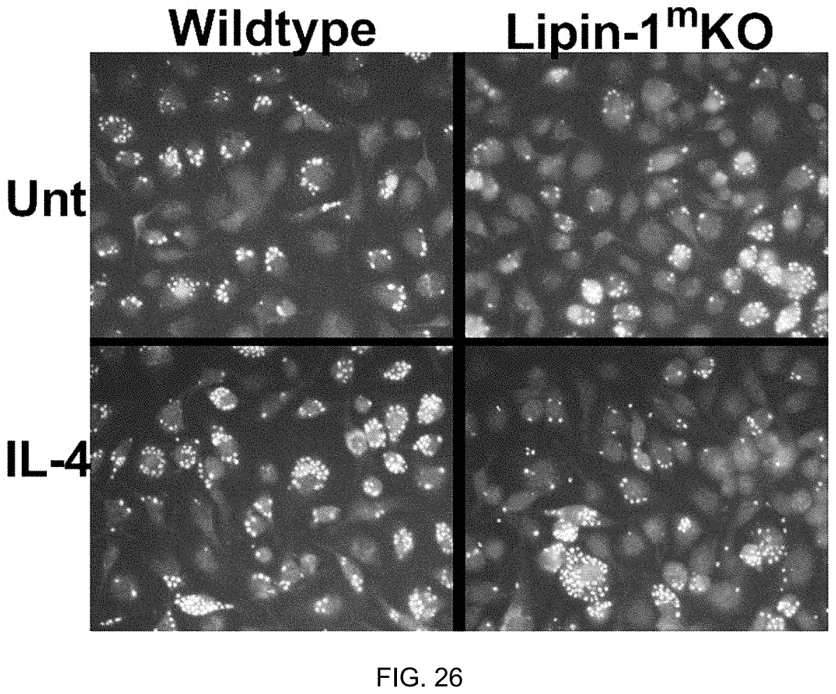

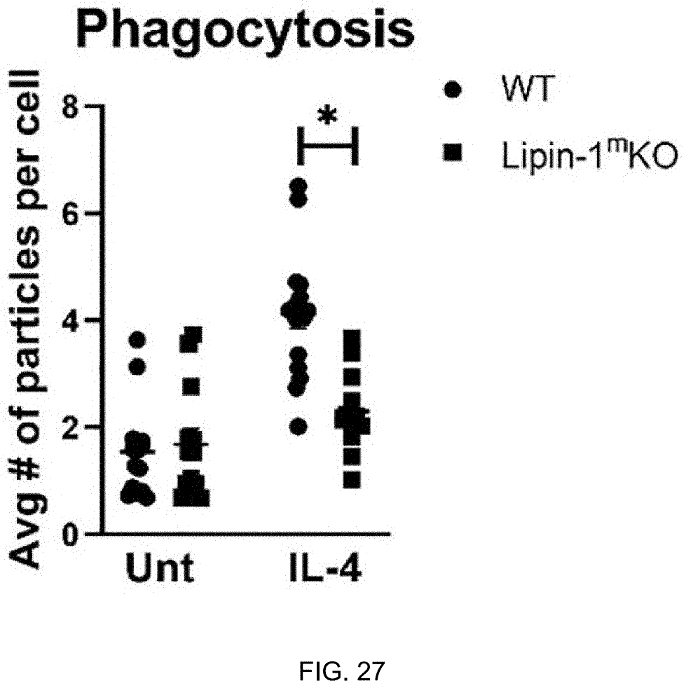

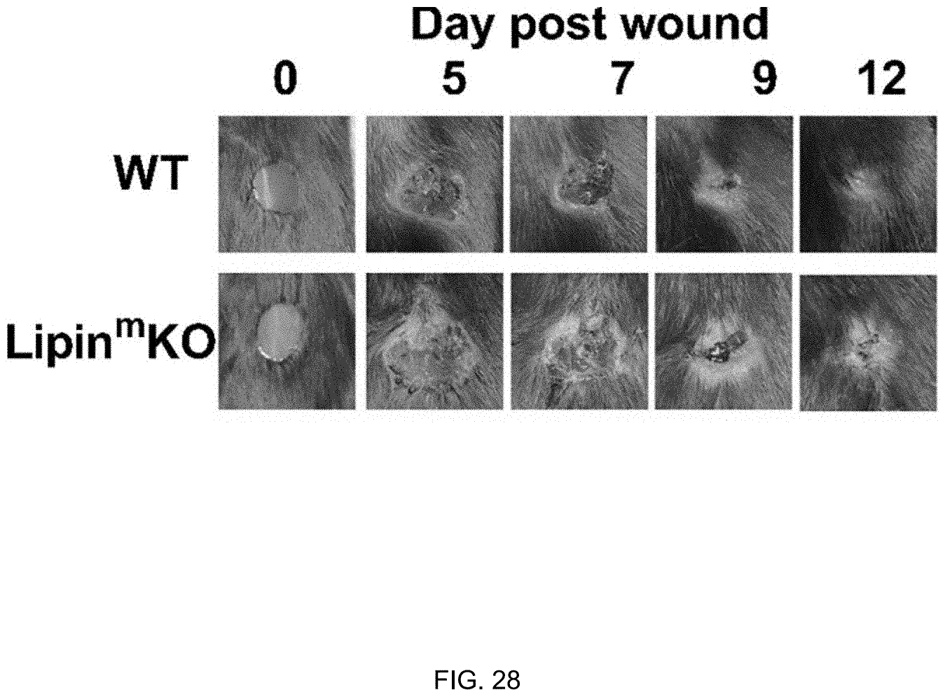

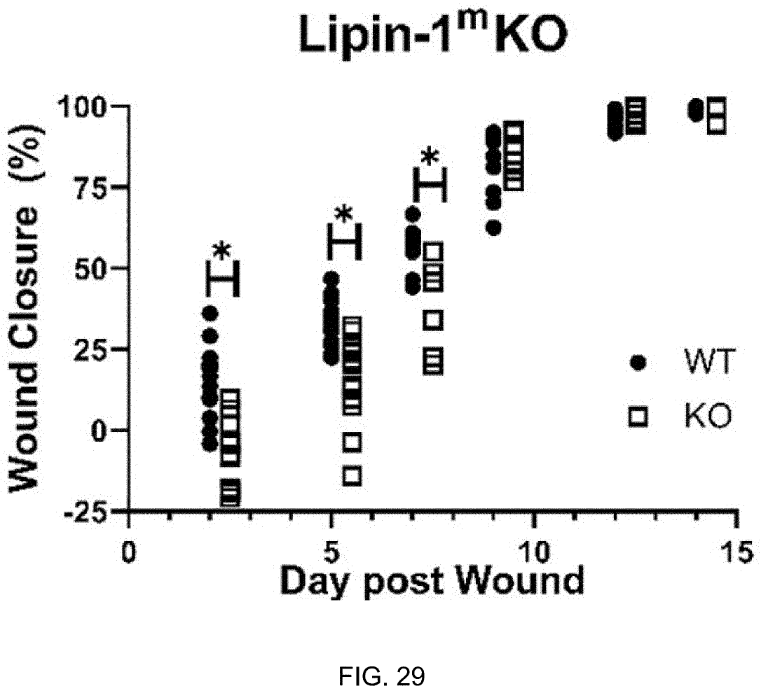

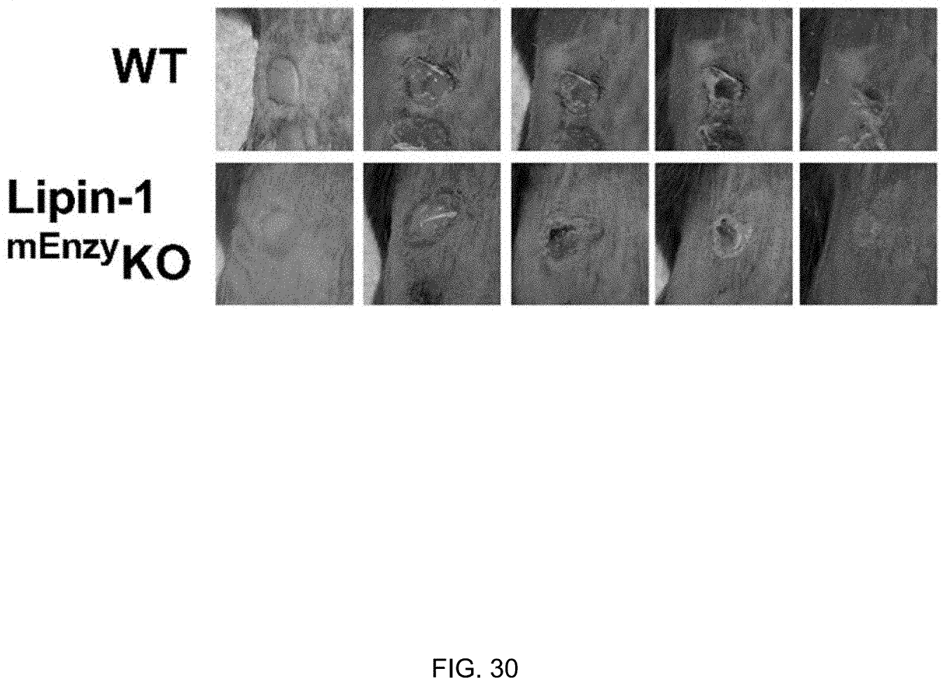

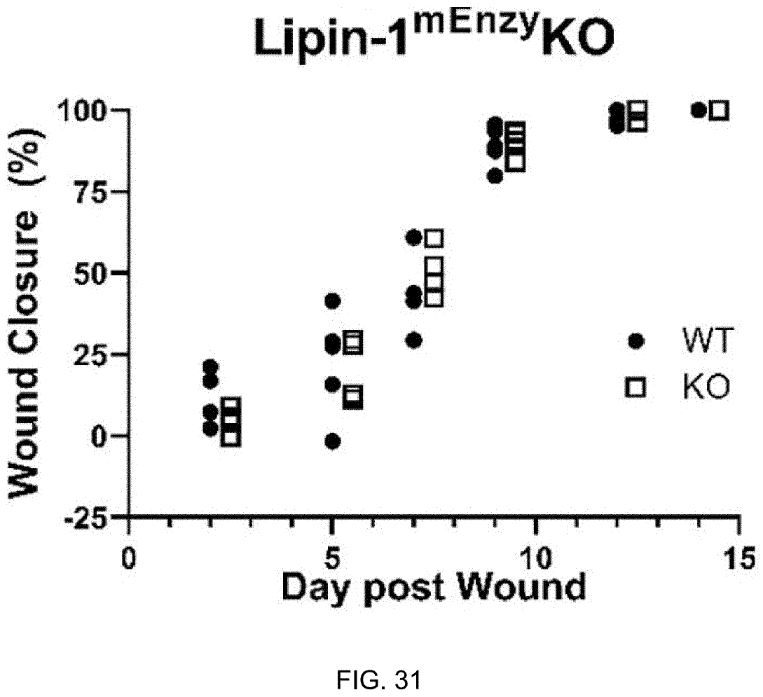

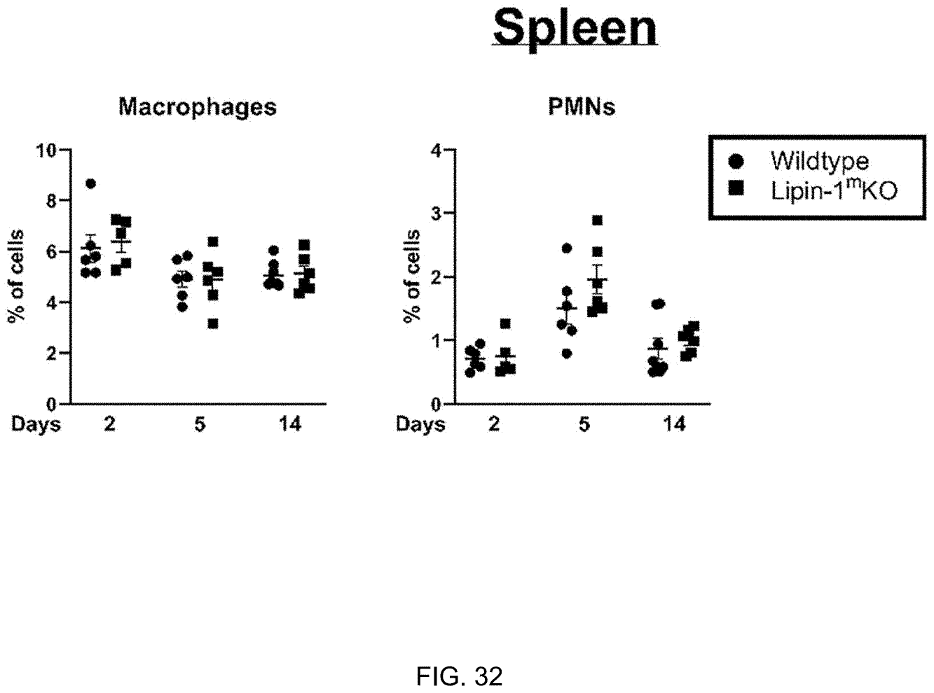

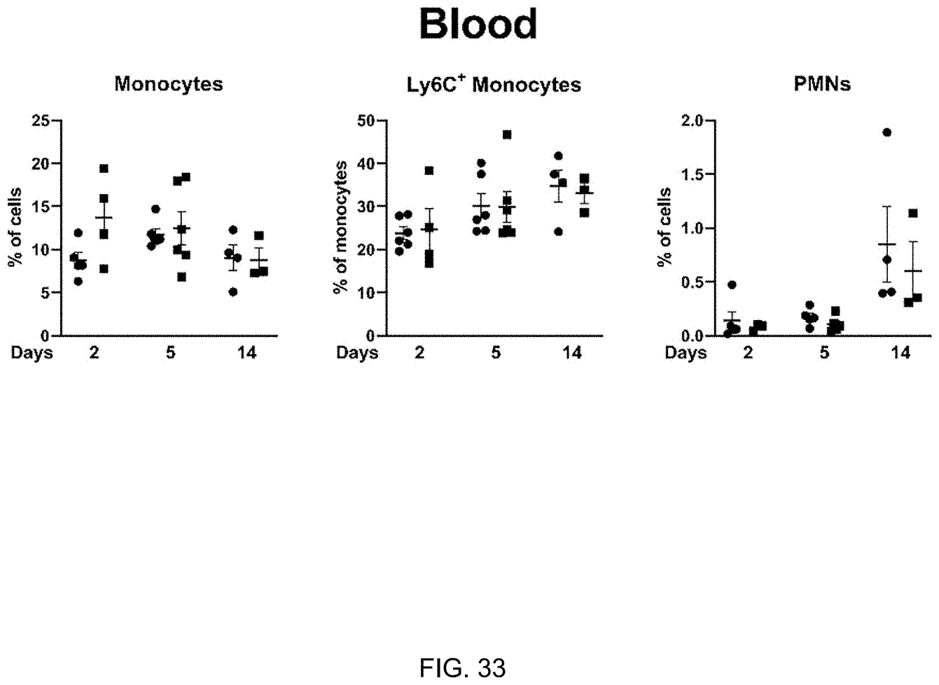

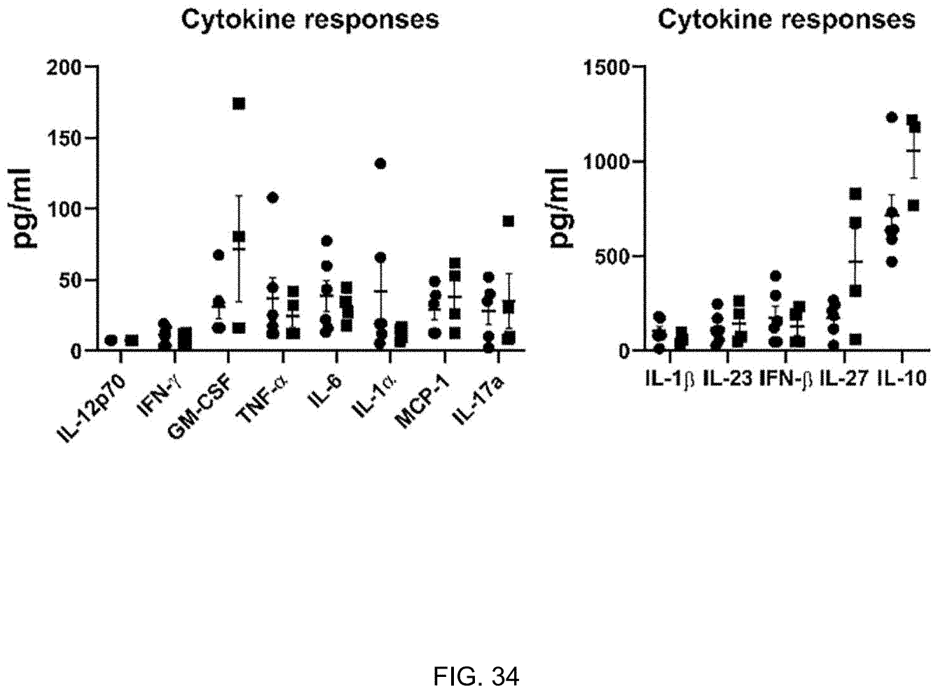

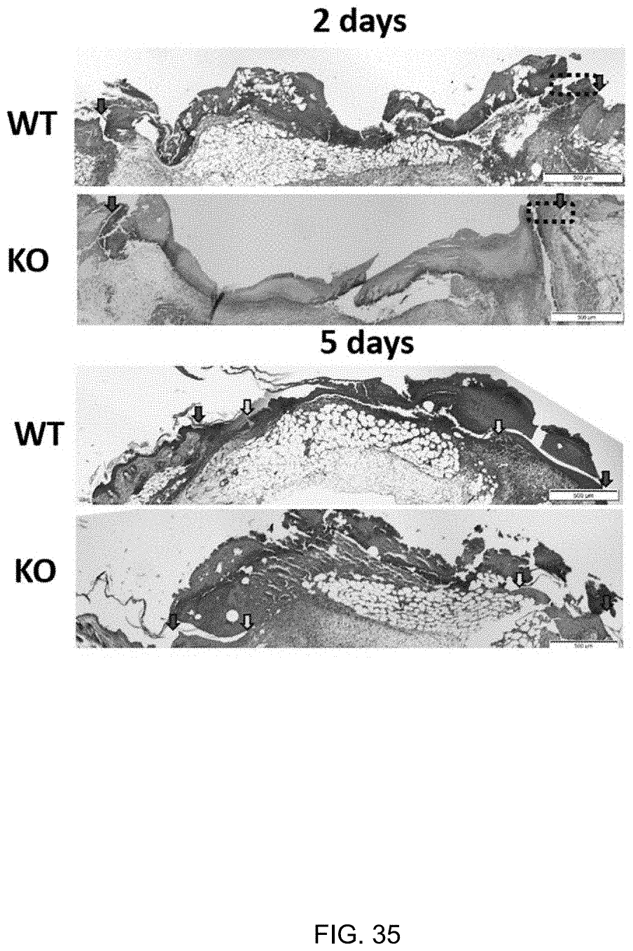

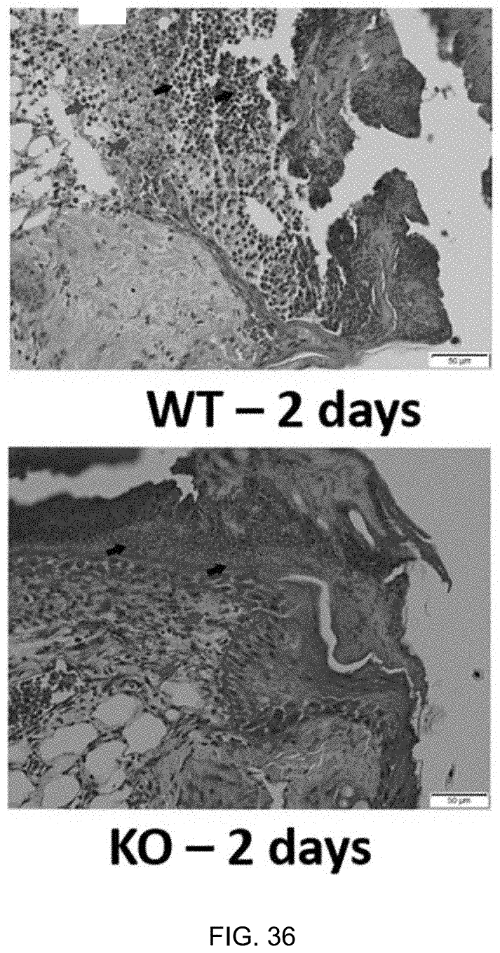

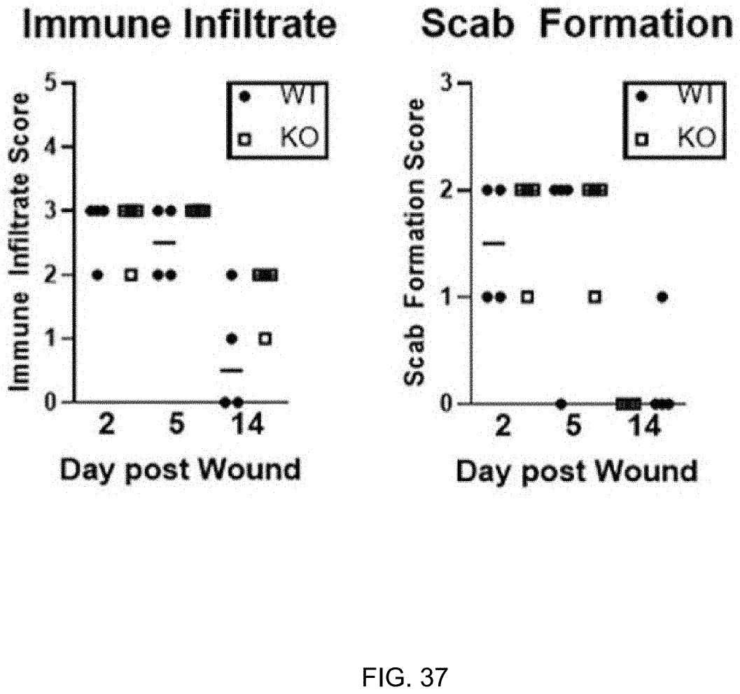

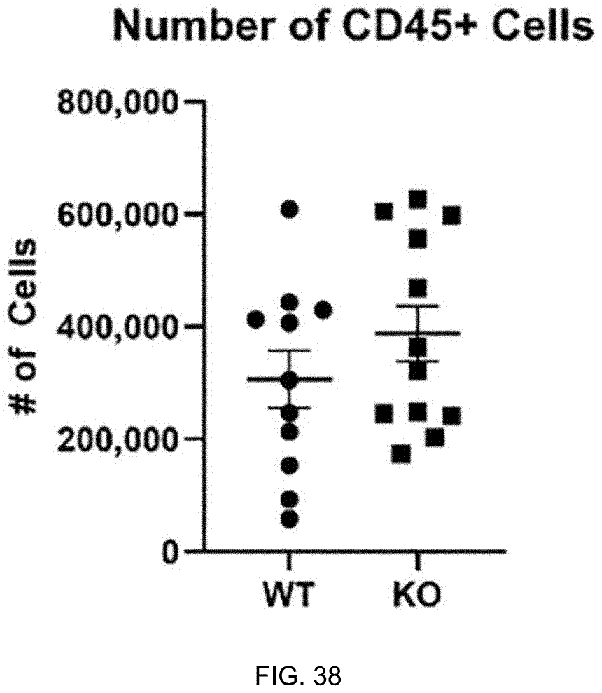

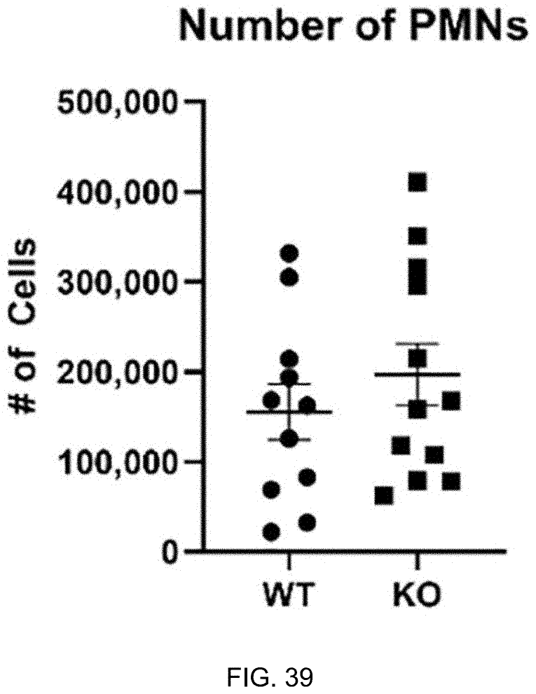

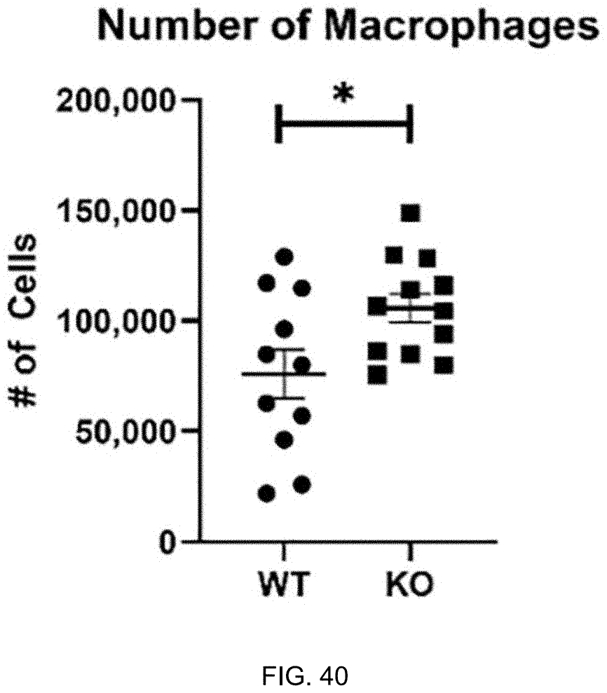

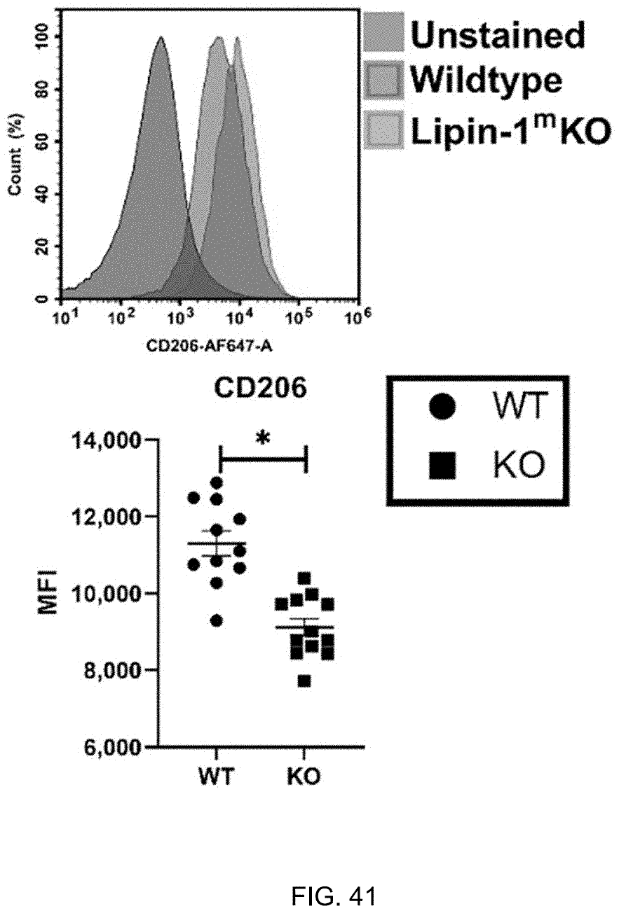

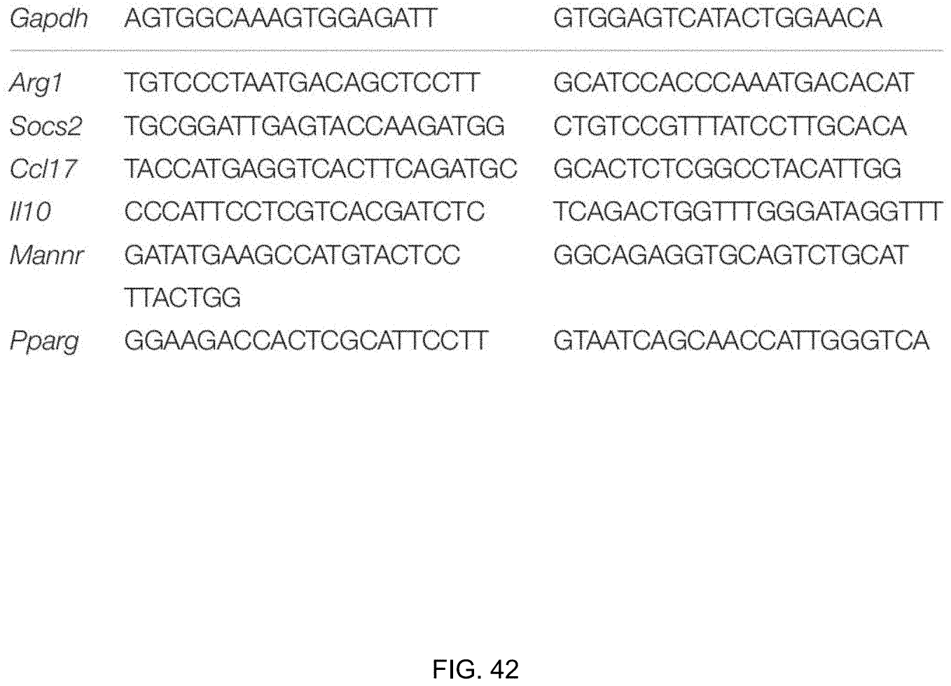

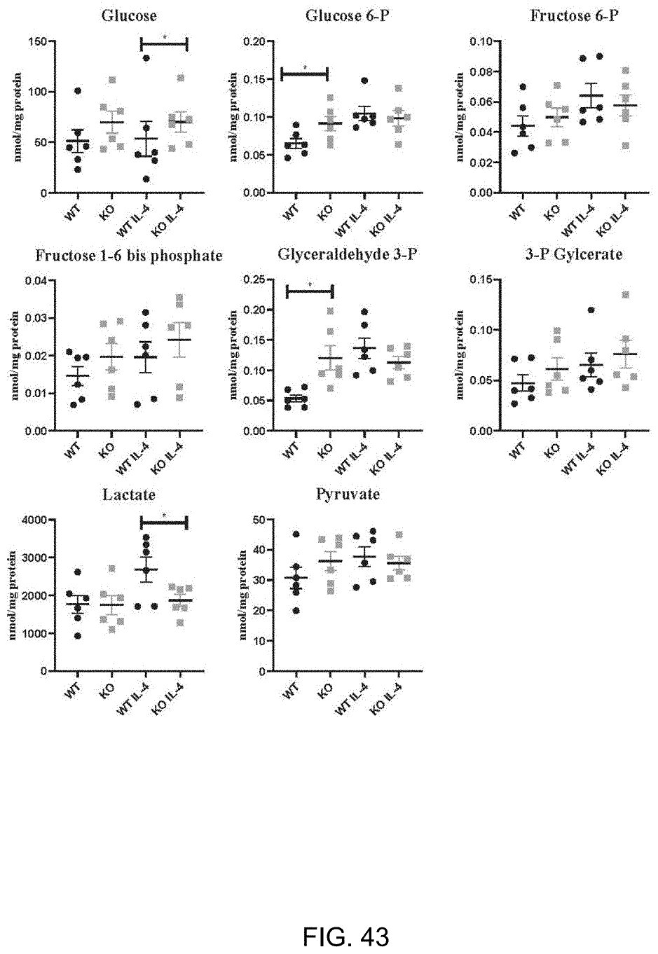

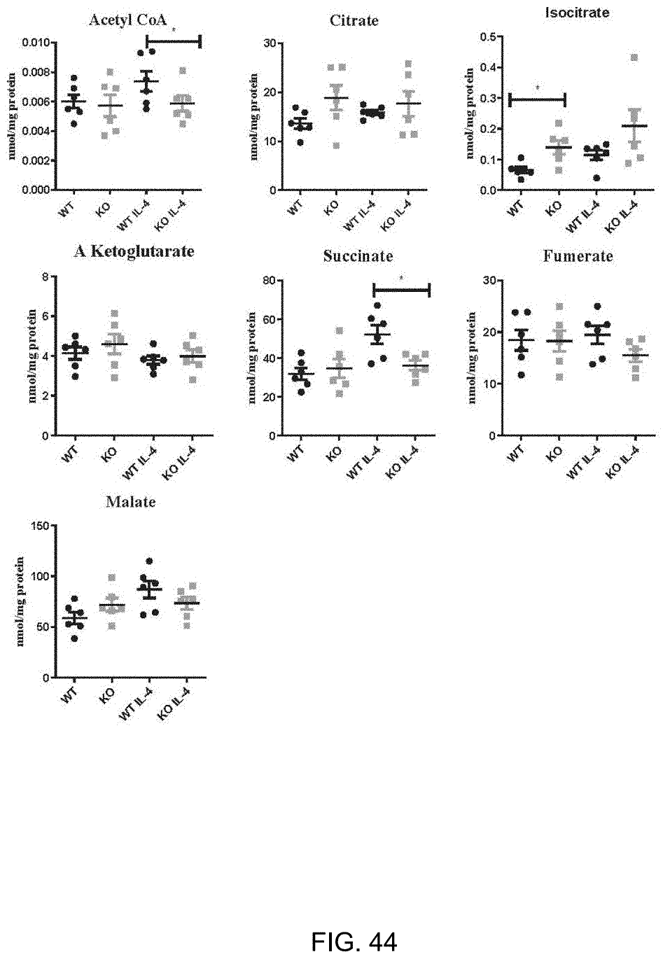

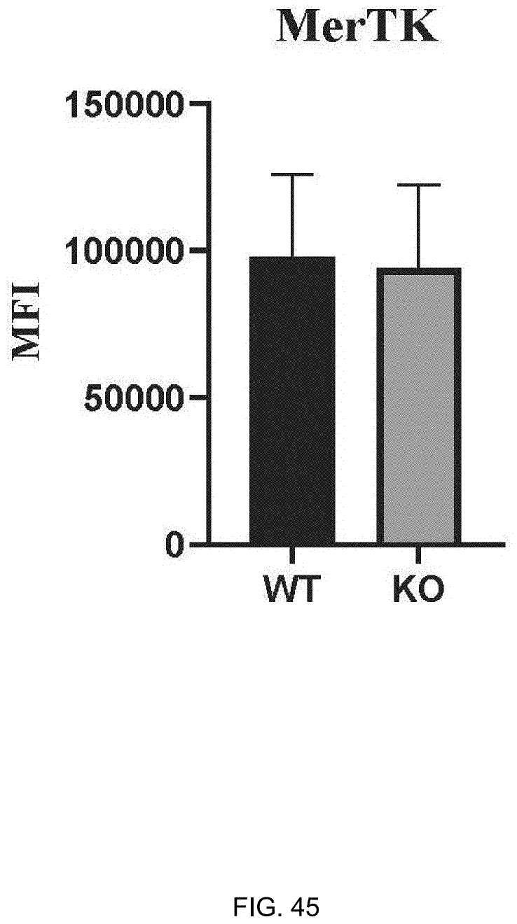

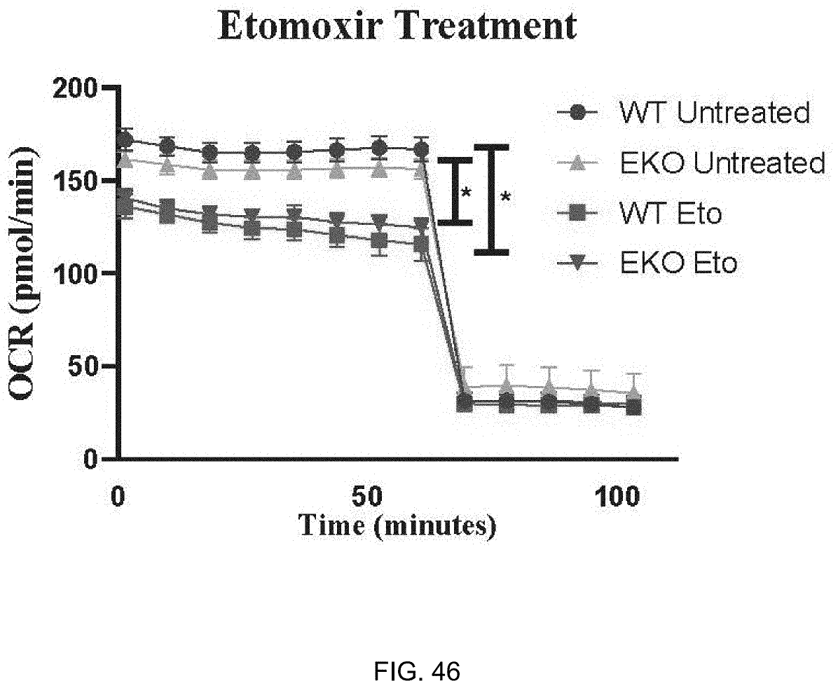

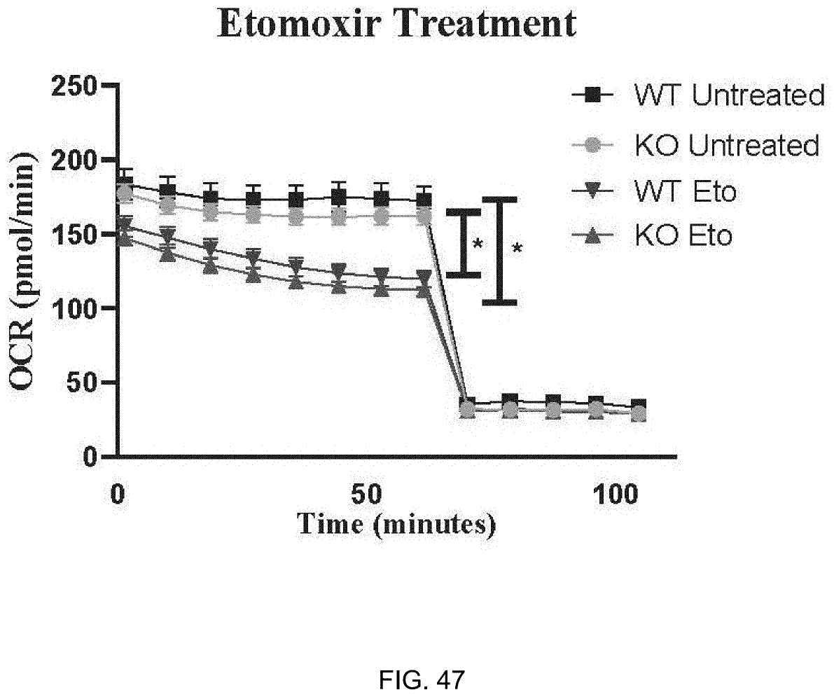

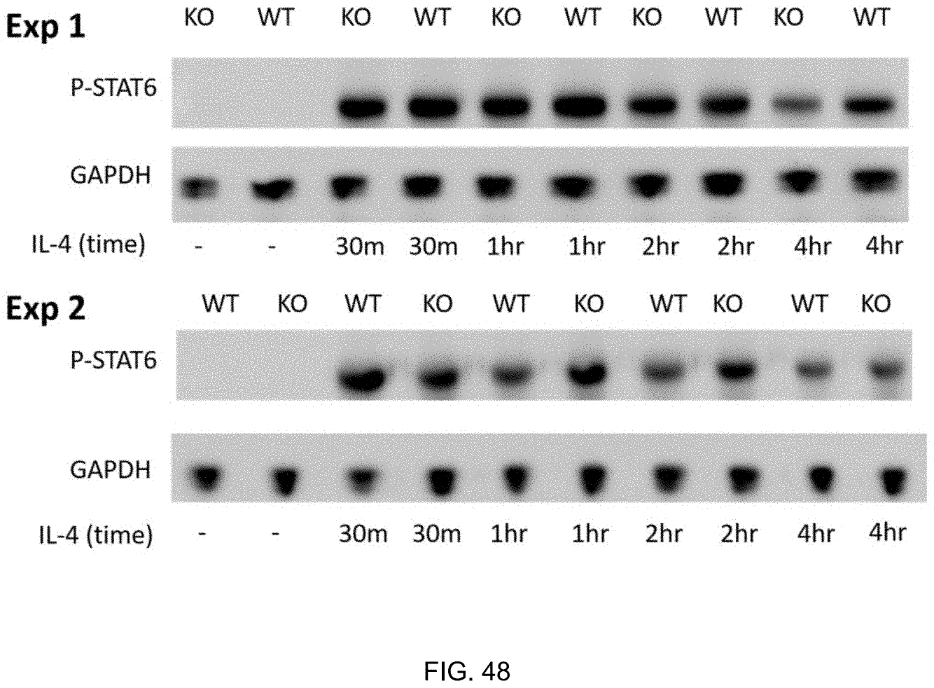

FIG. 6 shows basal and maximal (FCCP) OCR of WT and EKO BMDMs treated with and without IL-4. n=3. Graphed data represent mean OCR with SEM. *p≤0.5.

FIG. 7 shows lipin-1 is required for use of free fatty acids. BMDMs isolated from WT and KO mice were treated with 40 ng/ml for 1.5 h. Immediately before assay, 100 μM palmitate was added to requisite wells, and OCR was analyzed via Seahorse extracellular flux assay. Area under the curve was analyzed via one-way ANOVA. n=3. Graphed data represent mean OCR with SEM. *p≤0.5.

FIGS. 8 and 9 show lipin-1 does not regulate lipid uptake. BMDMs isolated from WT and KO mice were treated with 100 μM BODIPY palmitate for 1 h at either 37° C. or at 4° C. BMDMs (CD11b⁺, F4/80⁺, FITC⁺, and Ly6G⁻) were analyzed via flow cytometry to determine MFI.

FIG. 8 shows a representative histogram of WT and KO BMDMs treated with BODIPY palmitate.

FIG. 9 shows compiled MFI with SEM of WT and KO BMDMs treated with BODIPY palmitate. n=3. Data were analyzed via a Student t test.

FIGS. 10-12 show lipin-1 regulates macrophage metabolism. Central carbon analysis of WT and KO BMDMs treated with and without 40 ng/ml IL-4 for 4 h (FIG. 10). Values represent fold change compared with WT control. Quantitative analysis of NADH (FIG. 11) and NADPH (FIG. 12) levels in WT and KO BMDMs treated with and without 40 ng/ml IL-4 for 4 h. Graphed data represent mean metabolite concentration with SEM. Student t test was performed as statistical analysis. n=6. *p≤0.5.

FIGS. 13-16 show lipin-1 contributes to efferocytosis.

FIG. 12 shows WT and KO mice subjected to an excisional wound-healing model. Number of macrophages/dead cells was analyzed via flow cytometry.

FIG. 14 shows BMDMs from WT and KO mice were subjected to a dual-label in vitro model of continuing efferocytosis, and percentage of macrophages (CD11b⁺ and F4/80⁺) that took up an initial event (primary) and multiple apoptotic bodies (continuing) were analyzed via flow cytometry. WT, EKO (FIG. 15), and KO (FIG. 16) mice were subjected to a zymosan model of peritonitis followed by peritoneal injection of PHK26-labeled AC. Percentage of macrophages (CD11b⁺, F4/80⁺, PKH26⁺, and Ly6G⁻) with labeled AC were analyzed via flow cytometry. Graphed data represent mean uptake with SEM. Student t test was used to analyze data. n=8-12. *p≤0.5.

FIGS. 17 and 18 show lipin-1 enzymatic activity does not contribute to AC-derived lipid use. OCR of WT (FIG. 17) and EKO (FIG. 18) BMDMs pretreated with and without 40 μM etomoxir for 15 min followed by the addition of AC at a 4:1 ratio. Graphed data represent mean OCR with SEM. n=3. *p≤0.5.

FIGS. 19 and 20 show lipin-1 transcriptional coregulator function is required for degradation of AC-derived lipids. OCR of WT (FIG. 19) and KO (FIG. 20) BMDMs pretreated with and without 40 μM etomoxir for 15 min followed by the addition of AC at a 4:1 ratio. Graphed data represent mean OCR with SEM. n=3. *p≤0.5.

FIGS. 21-23 show lipin-1 promotes IL-4 mediated gene expression. In FIG. 21, flow cytometry was used to quantify CD 11b surface expression of BMDMs from lipin-1^{m}KO and littermate controls. Each dot represents an independent experiment. In FIG. 22, lipin-1 was quantified by Western blot analysis, representative image of three independent experiments shown. In FIG. 23, BMDMs generated from lipin-1^{m}KO, lipin-1^{mEnzy}KO and their respective littermate control mice. BMDMs were stimulated with 20 ng/ml IL-4 for 4 h. mRNA was isolated and wound healing associated genes were quantified by qRT-PCR. No difference was noted between littermate controls as such they were combined in WT. Each dot represented an individual experiment. Experiments were performed a minimum of three times. All data were normal except for WT IL-4 treated gene expression. Mann Whitney test was used for comparing WT and lipin-1^{m}KO; unpaired T test used for comparing lipin-1^{m}KO and lipin-1mEnzyKO Data presented is mean±SEM, * p<0.05.

FIGS. 24 and 25 show lipin-1 does not regulate IL-4 mediated STAT6 phosphorylation. In FIG. 24, flow cytometry was used to quantify surface expression of IL-4R in unstimulated BMDMs from lipin-1^{m}KO and litter mate controls (mean±SEM n>3 from 3 independent experiments). In FIG. 25, BMDMs from lipin-1^{m}KO and litter mate controls were stimulated with 20 ng/ml IL-4 for 30 min. Protein was isolated and p-STAT6, STAT6 was quantified by Western blot analysis. A representative blot from three independent experiments is shown.

FIGS. 26 and 27 show lipin-1 contributes to IL-4 enhancement of phagocytosis. In FIG. 26, representative microscopic images of BMDMs from lipin-1^{m}KO and littermate control mice fed pHrodo-Green zymosan particles. In FIG. 27 quantification of number of beads (zymosan beads) divided by number of nuclei in a given image. Experiment was performed 3 times with 4 random image panels taken per group for a total of 12 images. Each dot represents analysis of a single image (mean±SEM n=12, *p<0.05).

FIGS. 28-31 show loss of full lipin-1 delays wound closure.

FIGS. 28 and 30 show representative images of gross lesions.

FIGS. 29 and 31 show percent wound closure as [(area of original wound−area of current wound)/area of original wound]×100. Wound measurements were made on days 0, 2, 5, 7, 9, 12, and 14 post-wounding. KO mice are shifted by a half day in graph in order to see differences (Experiment was performed a minimum of three times and each dot represents a single animal) *p<0.05. Each symbol represents an individual mouse.

FIGS. 32-34 show loss of lipin-1 does not alter systemic immune responses. Splenocytes (FIG. 32) and blood cells (FIG. 33) were stained with a panel of antibodies to quantify monocyte/macrophage and PMN populations. Myeloid populations were defined as CD45⁺, CD3⁻CD19⁻CD11b⁺. PMNs were CD11b⁺Ly6 g⁺ and monocytes were CD11b⁺Ly6G⁻. Each dot represents an individual animal. Experiment was performed twice (mean±SEM). In FIG. 34, cytometric bead arrays to quantify cytokine concentrations in serum taken from mice 2 days after wounding Each dot represents an individual animal. Experiment was performed twice (mean±SEM).

FIGS. 35-37 show loss of full lipin-1 delays wound closure. In FIG. 35, H&E depicting epithelial closure of 5 mm wounded skin in lipin-1mKO and wild type mice post 2 and 5 days of wounding. Red arrow heads indicate host tissue-wound interphase; yellow arrow heads indicate tip of epithelial tongue.

FIG. 36 shows representative micrographs depicting immune infiltration at the host tissue-wound interphase (highlighted in 5A in rectangular box) post 2 days of wounding. Red arrow points toward monocytes and black arrow points toward neutrophils.

FIG. 37 is pathology score concerning immune cell infiltration and scab thickness. Tissue from 4 mice from each group (n=4). Scale bar for FIG. 35 is 500 μm;

FIG. 36 is 50 μm.

FIGS. 38-41 show loss of full lipin-1 leads to reduce CD206 surface expression on macrophages within the wound. Cells were isolated from wounded tissue. Cells were stained with a panel of antibodies to identify leukocytes (FIG. 38), neutrophils (FIG. 39), and macrophages (FIG. 40). The inventors also used an anti-CD206 antibody to characterize macrophage polarization within the wound.

FIG. 41 is a representative staining of macrophages from the wound and quantification of mice. Each dot represents an individual wound from 6 animals per group. The experiment was performed twice (n=12) (mean±SEM). Data was tested for normalcy and T test was used for analysis.

FIG. 42 is a list of primers used for Quantitative Real Time PCR.

FIGS. 43 and 44 show lipin-1 coordinates metabolism within macrophages. Central carbon analysis of glycolytic intermediates (FIG. 43) and TCA cycle intermediates (FIG. 44) from WT and KO BMDMs treated with and without 40 ng/mL IL-4 for 4 hours. Graphed data represents mean metabolite concentration with standard error of the mean. N=3. * indicate p≤0.05.

FIG. 45 shows lipin-1 does not regulate MerTK cell surface expression. WT and KO mice were subjected to a zymosan model of peritonitis. Flow cytometry analysis of macrophage (CD11b+, F4/80+, MerTK+, and Ly6G−) MerTK was performed on peritoneal lavages. Graphed data represents mean MFI with standard error of the mean. N=3.

FIGS. 46 and 47 show equivalent levels of basal lipid utilization. BMDMs from WT, EKO (FIG. 46), and KO (FIG. 47) mice were treated with and without etomoxir for 20 minutes. Seahorse analysis was performed to determine oxygen consumption rate (OCR). Graphed data represents mean OCR with standard error of the mean. N=3. * indicate p≤0.05.

FIG. 48 is a time course of Stat6 phosphorylation in response to IL-4.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

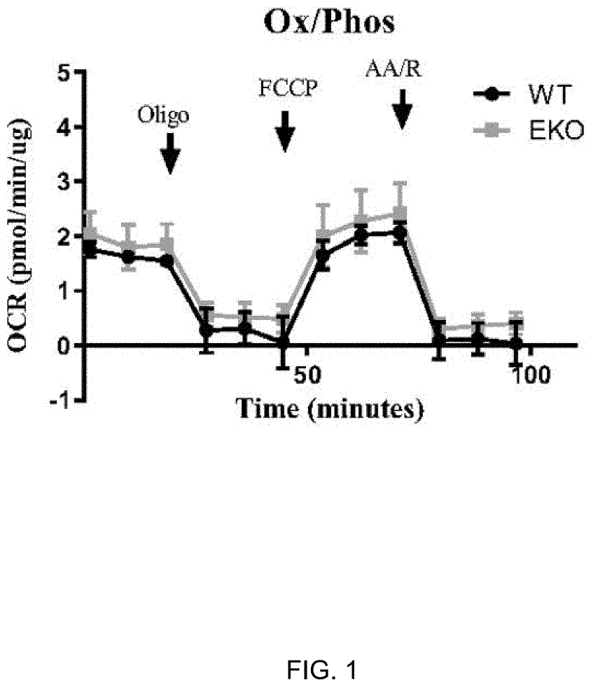

Turning now to FIGS. 1-48, a brief description concerning the various components of the present invention will now be briefly discussed.

Macrophages are innate immune cells that mediate tissue homeostasis by polarizing into unique phenotypes that range from pro-inflammatory to wound healing. Macrophage cellular responses restore normal tissue function. Defects in macrophage polarization can influence numerous disease pathologies including infectious disease, atherosclerosis, tumor growth, and impaired wound closure. Activation of macrophages via pattern recognition receptors (i.e., Toll like receptors) or through pro-inflammatory cytokine receptors (i.e., IFN-γ or TNF-α receptor) lead to pro-inflammatory activities. Conversely, IL-4, IL-10, IL-13, or TGF-β stimulation of macrophages promotes wound healing activities. The binding of these cytokines to their respective receptors leads to the activation and inhibition of numerous transcriptions factors that promote polarization of macrophages to a wound healing state. Most critical to wound healing polarization is the peroxisome proliferator-activated receptors (PPAR) family of transcription factors. PPAR activation via ligand binding and association with co-activators leads to both trans-repressive and transactivating activity. In macrophages, PPARs can transrepress NF-κB and STAT1 at the promoters of pro-inflammatory cytokines such as TNF-α. PPARs also promote the expression of genes associated with both lipid catabolism and macrophage wound-healing activity.

Lipin-1 belongs to the evolutionarily conserved three-member lipin family (lipin-1, -2, and -3) in mammals. Lipins enzymatically convert phosphatidate into diacylglycerol via dephosphorylation. Among lipin family proteins, lipin-1 exhibits the highest phosphatidate-specific phosphohydrolase activity. The inventors and others have shown that expression of a hypomorphic lipin-1 protein that lacks enzymatic activity attenuates pro-inflammatory macrophage responses by regulating glycerolipid synthesis. Lipin-1 enzymatic activity within macrophages contributes to disease pathogenesis of atherosclerosis, colitis, colon cancer, and LPS-induced inflammation. The overarching mechanism is likely due to lipin-1-mediated diacylglycerol production leading to protein kinase C and AP-1 transcription factor activation driving pro-inflammatory macrophage activities. In addition to acting as a lipid phosphatase, lipin-1 also independently acts as a transcriptional co-regulator by interacting with various DNA-bound transcription factors. It is unknown if lipin-1 transcriptional co-regulator activity is involved in regulating wound healing activity in macrophages. However, lipin-1 augments PPAR activity to promote adipogenesis in adipocytes and promotes beta-oxidation while suppressing very low-density lipoprotein production in hepatocytes. Lipin-1 also represses the activity of SREBP1, SREBP2, and NFAT4c by inhibiting the binding of these transcription factors to their respective promoters in hepatocytes. SREBP1, SREBP2, and NFAT4c have been identified to contribute to promotion of macrophage pro-inflammatory responses and inhibition of wound healing macrophage polarization. These studies suggest to the inventors that in macrophages the potential of lipin-1 transcriptional co-regulatory activity promoting PPARs and inhibiting SREBPs and NFAT4c might be important for the polarization of macrophages to a wound healing state. The inventors' data provides evidence that lipin-1 transcriptional co-regulator activity contributes to IL-4 mediated macrophage wound healing function.

### Materials and Methods

### Animals

All animal studies were approved by the LSU Health Sciences Center-Shreveport institutional animal care and use committee. All animals were cared for according to the National Institute of Health guidelines for the care and use of laboratory animals. After wounding, all mice were housed in individual filter-topped sterile cages, provided with sterile water and food ad libitum.

All animals used in this study were 8 to 10-week-old mice. Mice lacking lipin-1 enzymatic activity from myeloid cells (lipin-1^{mEnzy}KO) were generated. Briefly, mice with exons 3 and 4 of the Lpin1 gene flanked by LoxP sites (genetic background: C57BL/6J and SV129; generously provided by Brian Finck and Roman Chrast) were crossed with C57BL/6J LysM-Cre transgenic mice purchased from Jackson Laboratory (Bar Harbor, Me., United States). Exon 3 encodes the translational start site of lipin-1; however, deletion of this exon led to enforcement of an alternative start site causing expression of a truncated lipin-1 protein lacking 115 amino acids. The truncated protein lacks phosphatidic acid phosphohydrolase activity but retains cotranscriptional regulatory function. Mice fully lacking lipin-1 from myeloid cells (lipin-1^{m}KO) were generated by crossing mice with exon 7 of the Lpin1 gene flanked by LoxP sites [genetic background: C57BL/6J and SV129; generously provided by Brian Finck] with C57BL/6J LysM-Cre transgenic mice purchased from Jackson Laboratory (Bar Harbor, Me.). Deletion of exon 7 leads to frameshift, premature stop codon insertion, and a complete loss of lipin-1 protein. Age matched lipin-1^{flox/flox} littermate mice were used as controls.

### Excisional Wound Healing Model

Mice were anesthetized by 3% isoflurane (NDC, 14043-704-06) and clippers were used to remove hair from the dorsum. Exposed skin was disinfected with chlorohexidine swabs. Dorsal skin was folded, raised cranially, and mice were laterally positioned. Symmetric full thickness wounds were created using a sterile 5 mm biopsy punch (Integra). Gross images were taken and percentage of wound closure was assessed using a digital caliper at 0, 2, 5, 7, 9, 12, and 14-days post-wounding and expressed as [(area of original wound−area of current wound)/area of original wound]×100. After initial wounding, analgesic cream was applied to wounds (Aspercreme, Cattem, 0078940). Mice were routinely monitored for weight loss or any other type of distress until the end of the study.

### Generation of Bone Marrow-Derived Macrophages

Bone marrow-derived macrophages (BMDMs) were generated from lipin-1^{mEnzy}KO, lipin-1^{m}KO and littermate control mice as previously described. Briefly, femurs were excised under sterile conditions and flushed with Dulbecco's modified Eagle's Knock out medium (DMEM; Gibco, 10829) supplemented with 10% fetal bovine serum (Atlanta biologicals, S11150), 2 mM L-glutamax (Gibco-35050-061), 100 U/ml penicillin-streptomycin (Cell Gro, 30-604-CI), 1 mM sodium pyruvate (Cell gro, 25-060-CI), and 0.2% sodium bicarbonate (Quality biological, 118-085-721). Red blood cells were lysed using ammonium chloride-potassium carbonate (0.15 M NH₄Cl, 10 mM KHCO₃, 0.1 mM NA₂EDTA, adjusted to pH 7.2 and filter sterilized in 0.22 μm filter) lysis (ACK) followed by PBS wash. Isolated cells were incubated in sterile petri dishes for 7 to 10 days in BMDM differentiation medium—DMEM KO (Gibco10829) supplemented with 30% L-cell conditioned medium, 20% fetal bovine serum [Atlanta biologicals, 511150) 2 mM L-glutamax (Gibco-35050-061), 100 U/ml penicillin-streptomycin (Cell Gro, 30-604-CI), 1 mM sodium pyruvate (Cell Gro, 25-060-CI)], and 0.2% sodium bicarbonate (Quality biological, 118-085-721) at 37° C. and 5% CO₂. Once cells were 80% confluent, they were collected using 11 mM EDTA, pH 7.6 treatment. 10⁶ cells were seeded for RNA extraction and 5×10⁵ cells for protein isolation and flow cytometry analysis. After 4 h of seeding, cells were treated with 0 or 20 ng/ml IL-4 (R&D Biosystems, 404-ML-050) for various times.

### Flow Cytometry

### Interleukin-4 Receptor Staining

Bone marrow-derived macrophages were incubated with CD16/CD32 (e-Bioscience, 14-0161-86) for 20 min. BMDMs were then incubated with PECy7 conjugated anti-CD11b (e-Bioscience, 25-0112-81, clone M1/70), and PE conjugated anti-IL-4R (Biolegend, 144803) for 30 min in the dark. Cells were then fixed with 4% formaldehyde and analyzed using BD LSRII (San Jose, Calif., United States).

### Immune Composition Staining

Spleens were homogenized in FACS wash buffer (1% bovine serum albumin, 1 mM EDTA, and 0.1% sodium azide in phosphate buffered saline) followed by centrifugation at 300×g for 5 min. The supernatant was decanted and splenocytes were dislodged in 3 ml of ACK lysis buffer. Splenocytes were incubated on ice for 5 min. Splenocytes were washed in FACS wash buffer then centrifuged. The pellet was resuspended in FACS wash buffer and strained with a 40 μm cell strainer (Falcon, 352340), and counted. Splenocytes were adjusted to 1×10⁶ cells/mL in RPMI. Blood was collected in EDTA coated tubes. 100 μl of blood was lysed in 3 mls of ACK lysis buffer and then washed with FACS wash buffer. The entire sample of blood cells were stained. Splenocytes and blood cells were incubated with anti-CD16/CD32 (e-Bioscience, 14-0161-86) for 20 min. After blocking, cells were stained with a cocktail of antibodies: AF700 conjugated anti-CD45.2 (Biolegend, 109821, clone104), BV605 conjugated anti-CD3 (Biolegend, 100237, clone17A2), BV786 conjugated anti-CD11c (BD Biosciences, 563735, cloneHL3), PECy7 conjugated anti-CD11b (eBioscience, 25-0112-81, clone M1/70), PEe610 conjugated anti-CD19 (eBioscience, 61-0193-80, clone eBiolD3), FITC conjugated anti-Ly6G (BD Biosciences, 551460, clone1A8), PE conjugated anti-Ly6C (eBioscience, 12-5932-80, cloneHK1.4) and APC-Cy7 conjugated anti-CD115 (Biolegend, 135532, cloneAFS 98). Appropriate F Minus One Controls were used to correct background and exclude spectral overlap staining. Compensation control (Comp Bead, Invitrogen, 01-2222-42) were used. Flow cytometry analysis was performed using BD LSRII (San Jose, Calif.). Data analysis was done using FCS express (Denovo Software) and NovoExpress (AceaBio).

### Wound Staining

Quantification of macrophage phenotypes within the wound was performed as previously described. Briefly dorsal skin was carefully removed from the euthanized mice and placed onto filter paper. 10 mm×10 mm tissue specimen including the wounded area and adjacent tissue was made. Subcutaneous fat and muscle were removed from the tissue and wound was minced into 4-5 smaller pieces. Tissue was further digested in Dispase II enzyme cocktail (2 mg/mL, Thermo Fisher Scientific 17105-041 and 0.1-mg/mL DNase I Roche, cat. #10104159001) in a volume of 700 μL DMEM (Gibco10829) media and incubated in a shaker at 1,400 rpm, 37° C. for 2 h. After incubation, undigested debris was removed by filtering the sample through 70 μm strainer. Add 500 μL of cold FACS wash buffer to the side of strainer to wash off the remaining cells into the collection tube. Centrifuge at 4° C. for 5 min at 400×g. Remove the supernatant and resuspend the cell pellet in 200 μL cold FACS buffer. 0.5×10⁶ cells were then stained first for dead cells using Invitrogen aqua live/dead stain (Thermo Fisher Scientific L34965). After live/dead staining, cells were stained with anti-CD16/CD32 (e-Bioscience, 14-0161-86) for 20 min. After blocking, cells were stained with a cocktail of antibodies: AF700 conjugated anti-CD45.2 (Biolegend, 109821, clone104), PECy7 conjugated anti-CD11b (eBioscience, 25-0112-81, clone M1/70), FITC conjugated anti-Ly6G (BD Biosciences, 551460, clone 1A8), PE-Cy5 conjugated anti-F4/80 (Invitrogen, 15-4801-80, clone BM8), and AF647 conjugated anti-CD206 (Biolegend, 141711, clone C068C2). Appropriate F Minus One Controls were used to correct background and exclude spectral overlap staining. Compensation control (Comp Bead, Invitrogen, 01-2222-42) were used. Flow cytometry analysis was performed using Novocyte Quanteon (Aceo Bio). Data analysis was done using FCS express (Denovo Software) and NovoExpress (AceaBio).

### Western Blot

Cells were lysed in 1× NuPage LDS sample buffer [containing 100 mM dithiothreitol (DTT; Life Technologies), 1× protease inhibitor cocktail (Thermo Fisher Scientific), 1× phosphatase inhibitor cocktail 2 (Sigma Aldrich), and 1× phosphatase inhibitor cocktail 3 (Sigma Aldrich)]. Protein concentration was determined by Peirce™ 660 nm Protein Assay (Thermo Fisher Scientific) and 20 μg of each sample was separated using 4 to 12% polyacrylamide NuPAGE Novex gel (Invitrogen) run at 200 V for 55 min. Semidry transfer (Novex, SD1000) was performed for 45 min at 20 V onto a polyvinylidene difluoride (Immobilon-FL) membrane (EMD Millipore). The membranes were further blocked for 1 h at room temperature using Li-Cor blocking buffer (Li-Cor Biosciences) and incubated overnight with primary antibodies for Lipin-1 (CST #14906), P-STAT6 (CST #56554), STATE (CST #5397), and GAPDH (CST #2118). Goat anti-rabbit HRP secondary antibody (Jackson #111-035-144) was added to the membranes and incubated for 2 h at room temperature. Membranes were washed three times with tris buffered saline with tween 20 and incubated in ImmunoCruz Western blotting luminol reagent (Santa Cruz, sc-2048) for 1 min. Images were captured using an Amersham Imager 680 (GE Healthcare Bio-Sciences). Densitometry was performed using IQTL 8.1 (GE Healthcare Biosciences). Bands of interest were normalized to GAPDH.

### Quantitative Real Time PCR

Bone marrow-derived macrophages were treated with IL-4 (R&D Biosystems, 404-ML-050) for 4 h and mRNA was extracted from the cultured cells using RNeasy Mini Kit (Qiagen—74106) as per manufacturer's instructions. cDNA template was generated using qScript cDNA SuperMix (Quantabio, 95048). qRT-PCR was performed in a Biorad iCycler with SsoAdvanced Universal SYBER Green SuperMix (Biorad, 172-5271). Primers (FIG. 42) were obtained from the Harvard primer bank database. Primer specificity was confirmed using primer BLAST and by verifying the presence of a single peak in melt curve analysis. Results were expressed as fold change relative to IL-4 treated WT cells by 2^{-ΔΔCt} method after normalizing with GAPDH.

### Phagocytosis Assay

Bone marrow-derived macrophages (5×10⁵ cells) were cultured on sterile coverslips in culture wells and treated with 20 ng/ml IL-4 for 24 h. Culture medium was then replaced with DMEM, containing pHrodo™ green Zymosan A BioParticles® (Thermo Fisher Scientific, P35365) such that each well receives 0.1 mg zymosan particles. BMDMs were allowed to phagocytose for 1 h under dark incubation and then the assay was stopped by cold PBS wash. Cells seeded on coverslips were then fixed using 4% formaldehyde. Cells on cover slips were washed three times and then stained with DAPI slowfade (Invitrogen, S36938). Immunofluorescent images were taken using Olympus BX51 and evaluated using Image J (1.50a) analysis software. Phagocytic efficiency for each image was calculated by dividing the total number of fluorescent beads by the total number of nuclei in the fluorescent image, thus giving average number of beads per cell. Experiment was performed 3 times with 4 random images per group (n=12).

### Histology

Wound area was carefully excised at 2, 5, and 14 days after wounding and fixed in 10% neutral buffered formalin followed by paraffin embedding. 5 μm thick sections were cut from formalin-fixed paraffin-embedded tissue blocks. Sections were rehydrated, followed by hematoxylin-eosin (H&E) staining and dehydration. Stained sections were then imaged using Olympus BX51. 4× images were compared between each group to assess wound healing. Morphological score of inflammation: Evaluation of cellular infiltrate (polymorphonuclear and mononuclear cells) was done on H&E stained sections using the 10× objectives. The cells were counted at the wound bed and scored as 0, 1, 2, and 3 (absence of inflammation, Discrete-presence of few inflammatory cells, Moderate-many inflammatory cells and Severe-exaggerated inflammatory cellularity, respectively) for whole skin. The cellularity of the overlying crust or scab was excluded from the score. The scab was made of fibrin and polymorphonuclear cells. The scab was interpreted as either thin (scored as 1) or thick (Scored as 2) based on their morphological appearance on H&E sections. Scoring was performed in a blinded fashion.

### Cytometric Bead Array

Serum cytokine concentration was measured using Biolegend LEGENDplex (Biolegend Mouse inflammation Panel #740446). The assay was performed according to the manufacturer's instructions, and all samples were run in duplicate. Data was analyzed using the LEGENDplex Data Analysis Software.

### Statistical Analysis

GraphPad Prism 5.0 (La Jolla, Calif., United States) was used for statistical analyses. All data was tested for normalcy using the Shapiro Wilks Normalcy test. If data was normally distributed student T Test analysis was used for comparison between two data sets. If data was not normally distributed a Mann-Whitney test was performed. All other statistical significance was determined using a one-way ANOVA analysis of variance with a Dunnett's post-test. All in vivo experiments were performed a minimum of two times and all in vitro experiments were performed a minimum of three times. Figure legends provide specific details for each data set.

### Results

### Lipin-1 Contributes to IL-4 Elicited Gene Expression

Pro-inflammatory response in macrophages is influenced by lipin-1, but if lipin-1 contributes to wound healing responses by macrophages is unknown. The inventors have previously generated lipin-1^{mEnzy}KO mice that express a truncated lipin-1 protein lacking lipin-1 enzymatic activity but retain transcriptional co-regulatory function in myeloid cells. Here, the inventors generated lipin-1^{m}KO mice that lack the entire lipin-1 protein in myeloid cells. Comparing results between lipin-1^{mEnzy}KO mice and lipin-1^{m}KO mice allows the inventors to determine the contribution of lipin-1 enzymatic activity and infer the contribution of lipin-1 transcriptional coregulator activity on macrophage function. The inventors have previously demonstrated the ability to generate BMDMs from lipin-1^{mEnzy}KO mice and confirmed their phenotype. The inventors confirmed that the loss of full lipin-1 did not inhibit BMDM generation based on CD11b staining by flow-cytometry (FIG. 21). Western Blot analysis of proteins collected from cultured BMDMs demonstrated roughly an 85% reduction of lipin-1 protein in lipin-1^{m}KO BMDMs, residual lipin-1 protein is due to ineffective Cre excision of lipin-1 (FIG. 22). Having generated macrophages lacking lipin-1, the inventors investigated the contribution of lipin-1 to IL-4 mediated gene expression. BMDMs from lipin-1^{mEnzy}KO, lipin-1^{m}KO, and appropriate littermate controls were stimulated with 20 ng/ml of IL-4 for 4 h. mRNA was isolated and analyzed for the expression of several canonical wound-healing associated genes: Arg1, Socs2, Ccl17, Mannr, Il10, and Pparg. The inventors included littermate controls for both strains; however, no differences were noted between lipin-1^{m}KO and lipin-1^{mEnzy}KO littermate controls, therefore littermate controls were grouped together as wild type. Expression of several wound healing associated genes in wildtype, lipin-1^{mEnzy}KO and lipin-1^{m}KO BMDMs (FIG. 23) were comparable. However, IL-4 elicited gene expression was significantly lower in lipin-1^{m}KO BMDMs compared to either wild type or lipin-1^{mEnzy}KO BMDMs. These results demonstrate that lipin-1 enzymatic activity is dispensable for IL-4 mediated gene expression and suggests that lipin-1 transcriptional co-regulatory activity influences IL-4-mediated gene expression in macrophages.

Lipin-1 does not Influence Surface Expression of IL-4 Receptor or STAT6 Phosphorylation

Lipid membrane composition can influence the localization of receptors and/or signaling through those receptors. Lipin-1 is a regulator of glycerol lipid synthesis and the loss of lipin-1 may cause loss of either IL-4 receptor surface expression or signaling through the IL-4 receptor, thus resulting in impaired responses to IL-4. Flow cytometric evaluation of the surface expression of IL-4 receptor showed no difference between wild type and lipin-1^{m}KO BMDM (FIG. 24). Ligand binding of the IL-4 receptor-α (IL4Rα) triggers tyrosine phosphorylation at the cytoplasmic tail to facilitate recruitment and subsequent tyrosine phosphorylation of STAT6 by JAK1/JAK3 pathway. Wildtype and lipin-1^{m}KO BMDMs were stimulated with IL-4 for 30 min (FIG. 25), 1 and 4 h (FIG. 48) and protein was collected. Total STAT6 and phosphorylated STAT6 was measured by Western blot analysis. Similar levels of STAT6 phosphorylation was observed between wild type and lipin-1^{m}KO BMDMs (FIG. 25) These results show that defective IL-4 elicited gene expression in lipin-1^{m}KO BMDMs was likely not due to a failure in IL-4 binding to the IL-4 receptor and subsequent STAT6 phosphorylation.

### Lipin-1 is Required for Phagocytosis

The reduction in wound healing-associated genes in response to IL-4 suggests that lipin-1 contributes to macrophage wound healing function. Macrophages with a wound healing phenotype can have increased phagocytic capabilities. The inventors investigated the ability of BMDMs to phagocytize zymosan beads. The inventors mock treated or IL-4 treated BMDMs from lipin-1^{m}KO or litter mate controls for 24 h. The inventors then fed the macrophages pHrodo™ green Zymosan A BioParticles for 1 h. These particles do not fluoresce at 7.6 pH but do fluoresce at acidic pH, making it easier to identify internalized particles. The inventors then imaged using fluorescent microscopy and quantified average number of particles per cell. IL-4 stimulated lipin-1^{m}KO BMDMs had fewer particles per cell than wild type BMDMs (FIGS. 26 and 27). These results further implicate the importance of lipin-1 in macrophage function.

### Myeloid-Associated Lipin-1 Contributes to Wound Healing In Vivo

The inventors' in vitro studies suggest that lipin-1 contributes to IL-4 mediated macrophage polarization. The inventors next wanted to determine if these in vitro differences contribute to in vivo processes as well. Polarization of macrophages to a wound healing phenotype is required for proper wound closure in a full excision wounding model. The inventors decided to investigate if the loss of myeloid-associated lipin-1 would alter wound closure. The inventors performed full excision wounding on lipin-1^{mEnzy}KO and their respective littermate controls. The inventors monitored wound closure at early (day 2 and day 5), middle (day 7 and day 9), and late (day 12 and day 14) stages of wound healing. Lipin-1^{m}KO mice had an initial delay in wound healing (days 2, 5, and 7) as compared to litter mate controls (FIGS. 28 and 29). 9 days after wounding, wounds were of comparable size between lipin-1^{m}KO mice and litter mate controls. In contrast, lipin-1^{mEnzy}KO did not differ from littermate controls in wound healing at any stage of healing (FIGS. 30 and 31). These results demonstrate that lipin-1 enzymatic activity in myeloid cells is dispensable for full excision wound closure and suggests that lipin-1 transcriptional co-regulatory activity in myeloid cells influences full excision wound closure.

Lipin-1 Deletion does not Alter Myeloid Immune Composition

Loss of lipin-1 could potentially influence development of myeloid cells or myeloid mediated systemic responses. The inventors examined myeloid population in the spleen and blood to see if there were any alterations that may explain the delay in wound healing. The inventors isolated the spleen and blood at days 2, 5, and 14 post-wounding. Cells were isolated and stained with a panel of antibodies to quantify macrophages, monocytes, PMNs, and Ly6C⁺ monocytes. The inventors included Ly6C staining as Ly6C^{hi} and Ly6C^{lo} can both contribute to wound healing. The inventors observed no significant difference between lipin-1^{m}KO and litter mate control mice in any myeloid cell population analyzed in the blood or spleen (FIGS. 32 and 33). In addition to monitoring cellular responses, the inventors also examined mice serum cytokine concentration, 2 days post-wounding. The inventors chose day 2 as this day correlated with the biggest difference in wound size. No differences were noted in serum cytokine responses between lipin-1^{m}KO and littermate control mice (FIG. 34). These data suggest that myeloid associated lipin-1 activity that contributes to wound healing is likely mediated in the local environment rather than systemically.

### Loss of Lipin-1 Leads to Alteration in Wound Immune Composition

Impaired healing was prominent in the early stage of wound healing. Hence, further histopathological evaluation (FIG. 35) was performed by H&E staining in isolated wounds from lipin-1^{m}KO and littermate control mice at 2- and 5-days post wounding. On day 2, slightly interrupted superficial layer with void spaces were seen at the wound site; scattered mononuclear cells and neutrophils were also observed within the superficial layer in control mice. In Lipin-1^{m}KO mice, the superficial layer was poorly bridged with large void spaces with more inflammatory cells (FIG. 35). A very thick crest/scab was evident at the wound area which was highly infiltrated with mononuclear cells and neutrophils indicative of hyper inflammatory phase in Lipin-1^{m}KO mice. On day 5 epidermal tongue (depicted by yellow arrow heads) extended toward the center of the wound, indicative of wound bridging and healing in control mice. But, in lipin-1^{m}KO mice, the crest region was still thick with large number of immune infiltrates and they lacked a definitive epidermal closure and organization, suggestive of impaired healing. Wound closure (interphase between host tissue and wound depicted by red arrow heads) was also improved in the control mice. Scoring of the stained sections (0-3 inflammatory infiltrate and 0-2 crust thickness) by a blinded pathologist showed no significant difference in inflammatory recruitment in lipin-1^{m}KO mice (FIGS. 36 and 37).

The inventors wanted to further investigate whether loss of lipin-1 influence inflammation within the wound during early stage of healing and alter macrophage profiles. To analyze immune cells within the wound the inventors isolated 1 cm² skin including wound and surrounding tissue. Immune cells were isolated from digested skin and then phenotypically characterized by flow cytometry. As the inventors were looking at early time frame within the immune response to wound healing, the inventors concentrated on innate immune cells by quantifying the number of leukocytes (CD45+ cells), number of PMNs (CD45⁺, CD11b⁺, F4/80⁻, and Ly6G⁺), and number of macrophages (CD45⁺, CD11b⁺, and F4/80⁺). Although there was no significant difference in the total number of leukocytes or PMNs within the wounds, the inventors did see a significant increase in the number of macrophages within the wounds of lipin-1^{m}KO mice (FIGS. 38-40). These observations support the inventors' pathological scoring which showed no difference in immune infiltrate. The inventors next examined the surface expression of CD206 (Mannose receptor) on macrophages in the wounds to determine if loss of lipin-1 altered macrophage polarization. The inventors chose CD206 as it is well accepted as a marker for M2 polarization in vivo and the inventors had in vitro data demonstrating a significant reduction in MannR gene expression in lipin-1^{m}KO BMDMs. Macrophages from wounds of lipin-1^{m}KO mice had a significant reduction in surface expression of CD206 compared to WT mice (FIG. 41). These data suggest the importance of lipin-1 in macrophages during wound healing.

### Further Embodiment

Macrophages are innate immune cells that regulate tissue homeostasis and are critical for disease resolution. Macrophages are able to polarize toward distinct phenotypes, such as proresolving macrophages that aid in wound-healing processes or proinflammatory macrophages that clear pathogens. As macrophages polarize, they change their metabolic profile to effectively respond to stimuli. Proinflammatory macrophages predominately rely on glycolysis to generate ATP. Oxidative phosphorylation is reduced to generate reactive oxygen species and promote lipid synthesis. Proinflammatory macrophages have a break in the TCA cycle at two distinct points. The first break, a downregulation of isocitrate dehydrogenase, leads to the accumulation of citrate that is exported out of the mitochondria to be used in lipid synthetic pathways. The second break leads to the accumulation of succinate, which stabilizes HIF1α to increase expression of proinflammatory genes. Proinflammatory macrophages also increase the pentose phosphate pathway (PPP) to generate NADPH, which is used in lipid synthesis and cellular redox potential. Proresolving macrophages initially increase glycolysis to quickly generate ATP but heavily rely on oxidative metabolism to produce sufficient energy to carry out their functions. Proresolving macrophages use β-oxidation of fatty acids to produce acetyl-CoA, which is oxidized via a series of reactions in the TCA cycle. The oxidation of acetyl-CoA generates reducing equivalents, such as NADH, to fuel the electron transport chain. β-Oxidation has been shown to promote macrophage polarization toward the proresolving phenotype and is essential to disease resolution. Catabolism of apoptotic cell (AC)-derived lipids promotes IL-10 production and subsequent wound healing in a mouse model of myocardial infarction. However, how macrophages regulate lipid metabolism in response to proresolving stimuli to promote proresolving functions is not fully understood.

Lipin-1 is a phosphatidic acid phosphatase that converts phosphatidic acid into diacylglycerol to be used in lipid synthetic pathways. Lipin-1 also has enzymatically independent transcriptional coregulator activity in which lipin-1 binds to transcription factors, such as PPARs, to regulate their activity and subsequent gene expression. Lipin-1 transcriptional coregulator activity has been shown to contribute to the expression of genes that encode proteins involved in fatty acid transport and lipid catabolism in a variety of tissues and cells. Hepatic lipin-1 amplifies PGC-1α/PPARα regulatory pathway to stimulate β-oxidation. Lipin-1 not only controls fatty acid metabolism but may also regulate oxidative phosphorylation because genetic depletion of lipin-1 in skeletal muscle results in a significant decrease in oxidative phosphorylation. Loss of hepatic lipin-1 in mice results in an alteration in major metabolic pathways in a diurnal cycle, such as an increase in fatty acid synthesis and elevated peripheral glucose use. Genetic depletion of lipin-1 in Caenorhabditis elegans results in an increase in fatty acid synthesis genes, with a corresponding decrease in lipolytic genes. Individuals lacking lipin-1 have impaired β-oxidation during exercise, which can result in rhabdomyolysis. Collectively, these studies demonstrate the contribution of lipin-1 to lipid metabolism.

Macrophages balance their cellular metabolism to carry out their function and alterations in metabolic reprogramming prevent proper responses. Reduction in (3-oxidation negatively impacts macrophage function and its consequence on tissue restoration. The two independent molecular functions of lipin-1 appear to play contrasting roles in modulating macrophage polarization. Specifically, lipin-1 enzymatic activity promotes proinflammatory macrophage responses, whereas the nuclear activity promotes proresolving properties. Lipin-1-mediated diacylglycerol production promotes the activation of the AP-1 transcription factor, which leads to the production of proinflammatory cytokines IL-6, TNF-α, and IL-12 and proinflammatory eicosanoids such as PGE2. The enzymatic activity of lipin-1 in macrophages contributes to the pathogenesis of atherosclerosis, colitis, colon cancer, LPS-induced inflammation, and alcoholic liver disease. The inventors have further demonstrated that lipin-1 can also contribute to proresolving macrophage polarization (above). Specifically, the inventors' studies suggest that lipin-1 transcriptional coregulatory activity is required for promotion of macrophage proresolving/wound-healing phenotype. Mice lacking lipin-1 from myeloid cells have accelerated atherosclerosis and a defect in wound healing, which the inventors did not observe in mice lacking myeloid-associated lipin-1 enzymatic activity. However, the molecular mechanisms by which lipin-1 transcriptional coregulatory activity promotes macrophage proresolving phenotypic activity is unknown. The contribution of lipin-1 to β-oxidation in other tissues, taken together with lipin-1 involvement in polarization of macrophages toward a proresolving phenotype, led the inventors to hypothesize that lipin-1 regulates β-oxidation in macrophages stimulated with proresolving stimuli.

The inventors used a mouse model to genetically deplete either only the enzymatic activity of lipin-1 or completely deplete lipin-1 within myeloid-derived cells. This allows the inventors to elucidate which activity of lipin-1 contributes to lipid metabolism within macrophages. The inventors show that in macrophages, the transcriptional coregulatory activity of lipin-1 is required for increased oxidative metabolism in response to IL-4 and AC.

### Materials and Methods

### Animals

All animal studies were approved by the LSU Health Sciences Center—Shreveport institutional animal care and use committee. All animals were cared for according to the National Institutes of Health guidelines for the care and use of laboratory animals.

Mice lacking lipin-1 enzymatic activity from myeloid cells (lipin-1^{mEnzy}KO) were generated. Briefly, mice with exons 3 and 4 of the Lpin1 gene flanked by LoxP sites (genetic background: C57BL/6J and SV129; generously provided by B.N.F. and R. Chrast) were crossed with C57BL/6J LysM-Cre transgenic mice purchased from The Jackson Laboratory (Bar Harbor, Me.). Mice fully lacking lipin-1 from myeloid cells (lipin-1^{m}KO) were generated by crossing mice with exon 7 of the Lpin1 gene flanked by LoxP sites (genetic background: C57BL/6J and SV129; generously provided by B.N.F. with C57BL/6J LysM-Cre transgenic mice purchased from The Jackson Laboratory. Age-matched lipin-1 flox/flox littermate mice were used as controls.

### Generation of Bone Marrow-Derived Macrophages

Bone marrow-derived macrophages (BMDMs) were generated from lipin-1^{mEnzy}KO, lipin-1^{m}KO, and littermate control mice, as previously described. Briefly, femurs were excised under sterile conditions and flushed with D10: DMEM (Life Technologies) supplemented with 10% FBS (S11150; Atlanta Biologicals), 2 mM GlutaMAX (35050-061; Thermo Fisher Scientific), 100 U/ml penicillin-streptomycin (American Type Culture Collection), and 1 mM sodium pyruvate (HyClone). RBCs were lysed using ammonium chloride-potassium carbonate (0.15 M NH₄Cl, 10 mM KHCO₃, 0.1 mM NA₂EDTA, adjusted to pH 7.2 and filter sterilized in 0.22-μm filter) lysis (ACK) followed by PBS wash. Isolated cells were incubated in sterile flasks for 6 d in BMDM differentiation medium: DMEM knockout medium (11965-092; Life Technologies) supplemented with 30% L cell conditioned medium, 20% FBS (S11150; Atlanta Biologicals), 2 mM GlutaMAX (35050-061; Thermo Fisher Scientific), 100 U/ml penicillin-streptomycin (American Type Culture Collection), and 1 mM sodium pyruvate (HyClone) at 37° C. and 5% CO₂. Once cells were 80% confluent, they were collected using 11 mM EDTA (pH 7.6). BMDMs were resuspended in D10 for further experiments.

### AC Generation

Thymocytes (Jurkats) were subjected to UV light for 20 min in a 10-cm dish in sterile PBS. Cells were collected, and media was changed to D10 media followed by a 2-h incubation at 37° C. to allow for cells to become apoptotic. Cells were collected and centrifuged at 400×g for 5 min. Cells were resuspended in 2×10⁻⁶ M PKH26 for 3 min. Staining was stopped by adding equivalent volumes of FBS.

### Mitochondrial Bioenergetics

BMDMs collected from lipin-1^{mEnzy}KO, lipin-1^{m}KO, and littermate control mice were seeded at a density of 150,000 cells per well on XF24 cell culture microplates and allowed to incubate for 4 h. Experiments were conducted in XF assay medium containing 25 mM glucose, 2 mM 1-glutamine, and 1 mM sodium pyruvate and analyzed using a Seahorse XFe 24 extracellular flux analyzer (Agilent Technologies). Where indicated, the following were injected: ATP-synthesis inhibitor oligomycin (Oligo; 1 μM), carbonyl cyanide 4-(trifluoromethoxy)phenylhydrazone (FCCP) (2 μM) to uncouple ATP synthesis, rotenone (100 nM) to block complex I, and antimycin A (1 μM) (Sigma-Aldrich) to block complex III. Oxygen consumption rate (OCR) was analyzed using Wave Desktop software (Agilent Technologies).

### Palmitate Use

BMDMs collected from lipin-1^{mEnzy}KO, lipin-1^{m}KO, and littermate control mice were seeded at a density of 150,000 cells per well on XF24 cell culture microplates and allowed to incubate for 4 h. Macrophages were treated with 40 ng/ml IL-4 for 1.5 h. Media was then changed to nutrient-restricted media containing Agilent base media (100840-000) supplemented with 0.5 mM pyruvate (HyClone) and 0.5 mM GlutaMAX (35050-061; Thermo Fisher Scientific). Cells were then pretreated with 2 mM 2-deoxy-d-glucose (D6134; Sigma-Aldrich) for 15 min prior to the assay and maintained throughout. Directly before analysis, 100 μM palmitate was added, and OCR was assayed via the Seahorse extracellular flux analyzer (Agilent).

### MerTK Receptor Staining

Zymosan (0.1 mg) was injected i.p. into lipin-1^{m}KO and littermate control mice and allowed to incubate for 6 d. Mice were then sacrificed, and the peritoneal lavage was collected in FACS wash buffer (1% BSA, 1 mM EDTA, and 0.1% sodium azide in PBS). A total of 500,000 isolated peritoneal cells were blocked with CD16/CD32 (1:200) (14-0161-86; eBioscience) for 20 min. Cells were incubated with PECy7-conjugated CD11b (1:4000) (25-0112-81, clone M1/70; eBioscience), AF700-conjugated anti-CD45.2 (1:2000) (109821, clone 104; BioLegend), FITC-conjugated anti-Ly6G (1:800) (551460, clone 1A8; BD Biosciences), PECy5-conjugated anti-F4/80 (1:400) (15-4801-80, clone BM8; Invitrogen), and PE-conjugated anti-MerTK Ab (1:500) (151506; BioLegend) for 30 min in the dark. Cells were spun at 400×g for 5 min and resuspended in FACS fix (1% paraformaldehyde in FACS wash buffer). Appropriate F Minus One Controls were used to identify positive staining. Compensation controls (Comp Bead, 01-2222-42; Invitrogen) were used to exclude spectral overlap. Flow cytometry was performed using Quanteon flow cytometer (Acea Biosciences). Data analysis was performed using FCS express (Denovo Software) and NovoExpress (Acea Biosciences).

### Central Carbon Analysis

BMDMs from lipin-1^{m}KO and littermate control mice were seeded at a density of 1×10⁶/ml into Falcon tubes (60818-500; VWR). Cells were either treated with or without IL-4 (40 ng/ml) for 4 h. Cells were then collected and spun at 400×g for 5 min. Cell pellets were sent to Creative Proteomics (Shirley, N.Y.) for central carbon analysis.

### Lipid Uptake

Five hundred microliters of BMDMs from lipin-1^{mEnzy}KO, lipin-1^{m}KO, and littermate control mice were seeded at a density of 1×10⁶/ml in HBSS (SH30588.01; Thermo Fisher Scientific) containing 25 mM glucose into Falcon tubes (60818-500; VWR). BODIPY-labeled palmitate (D3821; Thermo Fisher Scientific) (100 μM) was added to macrophages and allowed to incubate for 1 h at either 37° C. or 4° C. Cells were then washed and resuspended in FACS wash, and mean fluorescent intensity (MFI) was analyzed via flow cytometry.

### In Vivo Efferocytosis

Lipin-1^{mEnzy}KO, lipin-1^{m}KO, and littermate control mice were injected i.p. with 0.1 mg of zymosan. Six days after zymosan injection, 4 million PKH26-labeled AC were injected into the peritoneal cavity of the mice and allowed to incubate for 45 min. Mice were then sacrificed, peritoneal lavages were collected, and isolated cells were blocked and stained with PECy7-conjugated anti-CD11b (1:4000) (25-0112-81, clone M1/70; eBioscience), AF700-conjugated anti-CD45.2 (1:2000) (109821, clone 104; BioLegend), FITC-conjugated anti-Ly6G (1:800) (551460, clone 1A8; BD Biosciences), and PECy5-conjugated anti-F4/80 (1:400) (15-4801-80, clone BM8; Invitrogen). Lavages were then analyzed via flow cytometry.

### In Vitro Continuing Efferocytosis

A total of 5×10⁵ BMDMs from lipin-1^{m}KO and littermate control mice were seeded into Falcon tubes. PK26-labeled AC were added at a 4:1 (AC/macrophage) ratio and allowed to incubate for 2 h. Cells were washed to remove unbound AC, then incubated with PKH67-labeled AC (4:1) for 45 min. Cells were washed and stained with PECy7-conjugated anti-CD 11b (1:4000) and PECy5-conjugated anti-F4/80 (1:400). Flow cytometry was performed to determine the ability of the macrophages to eat multiple AC.

### Statistical Analysis

GraphPad Prism 5.0 (La Jolla, Calif.) was used for statistical analyses. A Student t test analysis was used for comparison between two data sets. Area under the curve analysis followed by a one-way ANOVA was performed on palmitate and continuing efferocytosis Seahorse data. All other statistical significance was determined using a one-way ANOVA with a Dunnett posttest. All in vivo and in vitro experiments were performed a minimum of three times. Figure legends provide specific details for each data set.

### Results

### Lipin-1 is Required for Increased Metabolic Activity During Lipid Catabolic States

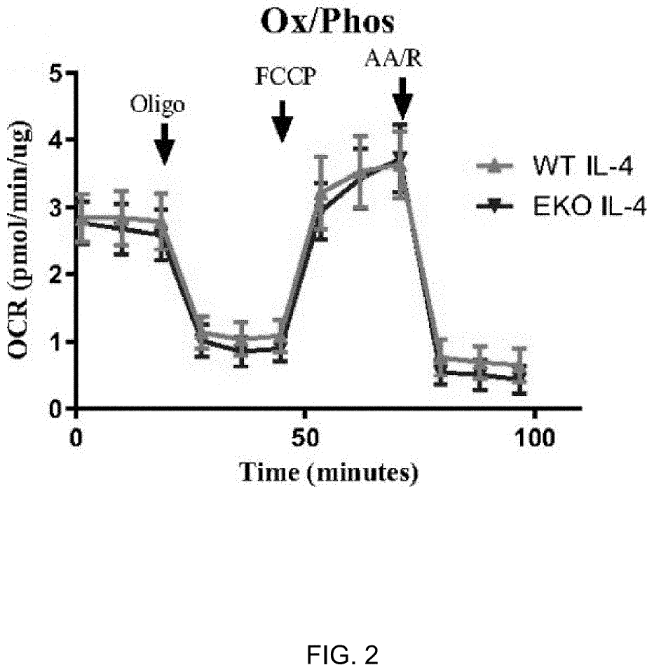

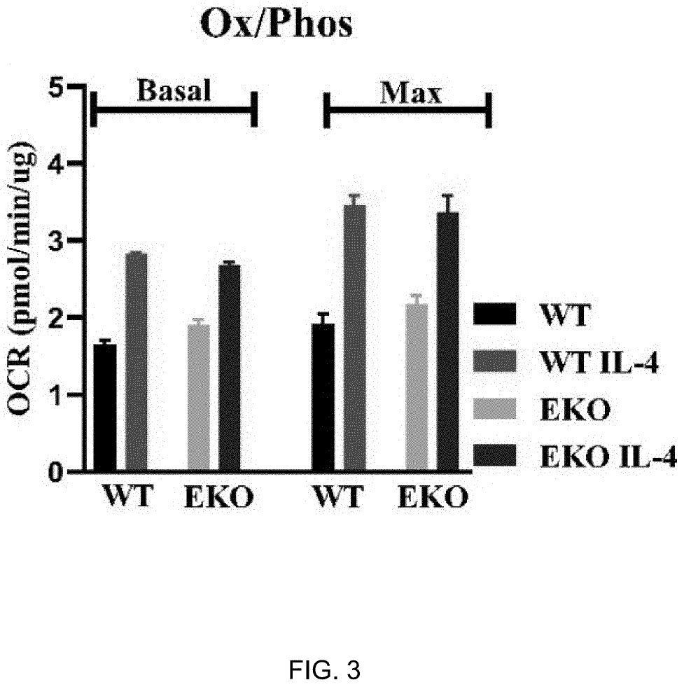

Lipin-1 enzymatic activity promotes macrophage proinflammatory responses, and the inventors have recently demonstrated that lipin-1 transcriptional coregulatory activity contributes to IL-4 elicited gene expression in macrophages. IL-4 stimulation polarizes macrophages toward a proresolving macrophage and elicits increased (3-oxidation and oxidative phosphorylation. In hepatocytes and skeletal muscle cells, lipin-1 promotes β-oxidation and oxidative phosphorylation. The inventors wanted to investigate if lipin-1 promoted oxidative phosphorylation in macrophages in response to IL-4. The inventors used BMDMs from lipin-1^{mEnzy}KO (EKO), lipin-1^{m}KO (KO), and littermate controls (wild type [WT]) to determine the contribution of the enzymatic activity and infer the contribution of the transcriptional coregulatory activity toward induction of oxidative phosphorylation. BMDMs from EKO, KO, and WT mice were stimulated with 40 ng/ml IL-4 for 4 h, and cellular bioenergetics were analyzed by Seahorse extracellular flux analyzer (Agilent Technologies). There were no differences in respiration between unstimulated WT and EKO macrophages (FIGS. 1 and 3). As expected, IL-4 stimulation increased OCR in WT BMDMs, and an equivalent increase in OCR was observed in EKO BMDMs (FIGS. 2 and 3). These data suggest that the enzymatic activity of lipin-1 is dispensable for IL-4-elicited oxygen consumption. The inventors next tested if lipin-1 transcriptional coregulatory activity contributes to oxidative metabolism in macrophages. WT and lipin-1 KO macrophages were stimulated with IL-4 for 4 h, and cellular bioenergetics were analyzed. There was no difference in respiration between unstimulated WT and lipin-1 KO BMDMs (FIGS. 4 and 6). Lipin-1 KO BMDMs failed to increase OCR in response to IL-4 stimulation, as seen in the WT BMDMs (FIGS. 5 and 6). The inability of lipin-1-deficient macrophages to increase OCR in response to IL-4, whereas macrophages lacking only lipin-1 enzymatic activity do respond to IL-4, suggests that lipin-1 transcriptional coregulator function is required for oxidative phosphorylation within IL-4-stimulated macrophages.

Lipin-1 Regulates Lipid Use within Macrophages

IL-4-stimulated macrophages shuttle fatty acids into the mitochondria to undergo β-oxidation and fuel oxidative phosphorylation. The loss of lipin-1 resulted in a failure of macrophages to increase oxygen consumption in response to IL-4, and the inventors hypothesize this defect is due to an inability to break down lipids for energy, leading to reduced oxygen consumption. To determine if lipin-1-deficient macrophages can use lipids to promote oxygen consumption, the inventors created an environment that is most favorable to using lipids for β-oxidation. The inventors stimulated WT and lipin-1 KO BMDMs with IL-4 for 1.5 h to elicit β-oxidation in nutrient- (pyruvate and glutamine) restricted media. The inventors inhibited glycolysis with the addition of 2-deoxy-d-glucose and added 100 μM palmitate to promote lipid-mediated oxygen consumption. OCR was measured via Seahorse XFe analyzer. The inventors observed no difference in oxygen consumption between IL-4-treated WT and lipin-1 KO BMDMs not treated with palmitate (FIG. 7). The addition of palmitate to WT BMDMs significantly increased OCR, suggestive of β-oxidation and oxidative phosphorylation (FIG. 7). Treatment of lipin-1 KO BMDMs with palmitate failed to increase OCR as seen in WT BMDMs (FIG. 7). These data suggest that lipin-1 is critical for the breakdown and use of lipids in response to IL-4. A potential explanation for the inability of lipin-1 KO BMDMs to increase oxygen consumption when fed palmitate is a defect in uptake. To address if lipin-1 affects palmitic acid uptake, the inventors treated WT and lipin-1 KO macrophages with IL-4 then fed them BODIPY-labeled palmitic acid and incubated for 1 h at either 4° C. or 37° C. to differentiate cellular binding from lipid uptake. Cells were analyzed via flow cytometry to determine 1MFI as a marker for palmitic acid uptake. There was no difference in MFI between WT and lipin-1 KO BMDMs (FIGS. 8 and 9). Taken together, these data suggest to the inventors that the loss of lipin-1 from macrophages is not inhibiting lipid uptake but rather lipid breakdown by β-oxidation.

### Lipin-1 Regulates Cellular Metabolism

The inventors' data suggest that lipin-1 is required for the breakdown and use of lipid in response to IL-4 stimulation. Macrophages need to align individual metabolic pathways to effectively respond to stimuli. The inventors wanted to understand the consequences of lipin-1 activity on immunometabolic responses in macrophages after stimulation with IL-4. WT and lipin-1 KO macrophages were stimulated with and without 40 ng/ml IL-4 for 4 h. Cell pellets were then sent to Creative Proteomics for central carbon analysis to determine the abundance of metabolites in major metabolic pathways, such as glycolysis, TCA cycle, and PPP. At baseline, lipin-1-deficient macrophages have an altered metabolism compared with WT macrophages. Lipin-1 KO BMDMs have multiple elevated glycolytic metabolites compared with WT BMDMs (FIGS. 10 and 43). These data suggest that the loss of lipin-1 increases glycolysis. The inventors also see an increase in metabolites within the PPP (FIG. 10). Upregulation of the PPP can result in increased NADPH (FIG. 10). There was also a significant increase in the TCA cycle metabolite isocitrate, an isomer of citrate, in lipin-1-deficient BMDMs compared with WT BMDMs (FIGS. 10 and 44). This suggests that there is a break in the TCA cycle of lipin-1-deficient BMDMs, which leads to the accumulation of isocitrate in lipin-1-deficient BMDMs.

Stimulation of WT BMDMs with IL-4 resulted in a significant increase in many glycolytic and TCA cycle metabolites with no break in the TCA cycle that is consistent with a proresolving phenotype (FIGS. 10 and 44). The alterations in metabolism in lipin-1-deficient BMDMs at baseline were exacerbated upon stimulation with IL-4. IL-4 stimulation of lipin-1 KO macrophages resulted in a significant increase in isocitrate, with a decrease in the TCA metabolite succinate (FIGS. 10 and 44). These data suggest a break in the TCA cycle that is phenotypically similar to proinflammatory macrophages, which have decreased β-oxidation and oxidative metabolism. The TCA cycle generates electron carriers (NADH) that are used by the electron transport chain to generate ATP as part of oxidative phosphorylation. The loss of lipin-1 resulted in a significant decrease in NADH levels compared with WT macrophages (FIG. 11), further supporting a break in the TCA cycle of lipin-1 KO macrophages. Lipin-1-deficient macrophages have an increase in the initial metabolites of the PPP, 6P-gluconate. The conversion of glucose-6P to 6P-gluconate generates NADPH, which can be used in lipid synthetic pathways (FIG. 10). The increase in initial metabolites of the PPP is further supported by significantly elevated NADPH levels (FIG. 12). These results suggest that lipin-1 regulates cellular metabolism in macrophages both basally and during IL-4 stimulation. Complete central carbon analysis can be found is found below:

Central Carbon Analysis of Untreated and IL-4 Treated BMDMs

| | WT Untreated | | | KO Untreated | | |
|---|---|---|---|---|---|---|
| Metabolite | Mean | SD | N | Mean | SD | N |
| Glucose | 51.17750 | 27.35046 | 6 | 69.96367 | 27.02348 | 6 |
| AMP | 1.38027 | 0.36445 | 6 | 1.64460 | 0.48146 | 6 |
| ADP | 0.54118 | 0.15461 | 6 | 0.68785 | 0.19382 | 6 |
| ATP | 0.19785 | 0.05512 | 6 | 0.24182 | 0.05098 | 6 |
| GMP | 11.49605 | 2.06806 | 6 | 15.53168 | 3.18161 | 6 |
| GDP | 0.09315 | 0.02086 | 6 | 0.11972 | 0.02965 | 6 |
| GTP | 0.03033 | 0.00543 | 6 | 0.03623 | 0.01280 | 6 |
| UMP | 11.59425 | 2.22809 | 6 | 15.84312 | 3.21107 | 6 |
| UDP | 0.19605 | 0.04795 | 6 | 0.21018 | 0.05687 | 6 |
| UTP | 4.72720 | 1.41691 | 6 | 4.81665 | 1.56620 | 6 |
| cyclic-ADPR | 0.26378 | 0.04475 | 6 | 0.34057 | 0.06694 | 6 |
| cyclic-AMP | 0.00155 | 0.00023 | 6 | 0.00215 | 0.00062 | 6 |
| cyclic-GMP | N/D | N/D | 6 | N/D | N/D | 6 |
| cyclic-UMP | 0.00267 | 0.00113 | 6 | 0.00365 | 0.00185 | 6 |
| 6P-Gluconate | 0.00952 | 0.00212 | 6 | 0.01673 | 0.00314 | 6 |
| Acetoacetyl-CoA | N/D | N/D | 6 | N/D | N/D | 6 |
| Acetyl-CoA | 0.00602 | 0.00114 | 6 | 0.00572 | 0.00181 | 6 |
| Acetylglucosamine-1P | 0.12357 | 0.02990 | 6 | 0.13195 | 0.02976 | 6 |
| Acetylglucosamine-6P | 0.34753 | 0.10287 | 6 | 0.40553 | 0.11507 | 6 |
| Acetyl-Phosphate | 0.34277 | 0.06492 | 6 | 0.45285 | 0.09686 | 6 |
| ADP-Glucose | 0.00257 | 0.00055 | 6 | 0.00305 | 0.00073 | 6 |
| DHAP | 0.03172 | 0.00814 | 6 | 0.04382 | 0.01648 | 6 |
| Erythrose-4P | 0.02757 | 0.00697 | 6 | 0.04807 | 0.02469 | 6 |
| Fructose-1P | 0.00295 | 0.00207 | 6 | 0.00542 | 0.00376 | 6 |
| Fructose-6P | 0.04412 | 0.01650 | 6 | 0.04982 | 0.01494 | 6 |
| Fructose-1,6-bisP | 0.01460 | 0.00620 | 6 | 0.01970 | 0.00870 | 6 |
| Glucosamine-6P | 0.03235 | 0.00806 | 6 | 0.04227 | 0.00890 | 6 |
| Glucose-1P | 0.02352 | 0.01006 | 6 | 0.03217 | 0.00968 | 6 |
| Glucose-6P | 0.06493 | 0.01601 | 6 | 0.09153 | 0.02314 | 6 |
| Glucose-1,6-bisP | 0.02112 | 0.00620 | 6 | 0.03325 | 0.01595 | 6 |
| Glyceraldehyde-3P | 0.05337 | 0.01434 | 6 | 0.12033 | 0.04953 | 6 |
| Glycerate-2,3P | 0.00272 | 0.00110 | 6 | 0.00133 | 0.00050 | 6 |
| Glycerate-2P | 0.00622 | 0.00230 | 6 | 0.00745 | 0.00295 | 6 |
| Glycerate-3P | 0.04750 | 0.01958 | 6 | 0.06135 | 0.02658 | 6 |
| Glycerol-3P | 0.64727 | 0.10200 | 6 | 0.99715 | 0.18017 | 6 |
| Hs-CoA | 0.04567 | 0.00881 | 6 | 0.04708 | 0.01047 | 6 |
| Malonyl-CoA | N/D | N/D | 6 | N/D | N/D | 6 |
| Mannose-6P | 0.00835 | 0.00275 | 6 | 0.01193 | 0.00340 | 6 |
| NAD+ | 0.27400 | 0.06681 | 6 | 0.35895 | 0.08451 | 6 |
| NADH | 0.24648 | 0.03568 | 6 | 0.39687 | 0.13417 | 6 |
| NADP+ | 0.10333 | 0.02168 | 6 | 0.12117 | 0.02339 | 6 |
| NADPH | 0.05775 | 0.01438 | 6 | 0.15060 | 0.06031 | 6 |
| PEP | 0.02818 | 0.01045 | 6 | 0.03588 | 0.01624 | 6 |
| Phosphocreatine | 0.16505 | 0.02658 | 6 | 0.26082 | 0.10873 | 6 |
| Ribose-1,5bisP | 0.00215 | 0.00079 | 6 | 0.00212 | 0.00047 | 6 |
| Ribose-5P | 0.04977 | 0.01576 | 6 | 0.07302 | 0.01405 | 6 |
| Ribulose-5P | 0.20308 | 0.06667 | 6 | 0.21937 | 0.05129 | 6 |
| Ribulose-1,5bisP | 0.01832 | 0.00429 | 6 | 0.02352 | 0.00382 | 6 |
| Sedoheptulose-7P | 0.03347 | 0.00912 | 6 | 0.04923 | 0.01551 | 6 |
| Succinyl-CoA | 0.01032 | 0.00233 | 6 | 0.01348 | 0.00441 | 6 |
| UDP-acetylglucosamine | 0.89497 | 0.21920 | 6 | 1.08968 | 0.28093 | 6 |
| UDP-Glucose | 0.39158 | 0.08451 | 6 | 0.49960 | 0.08908 | 6 |
| Acetoacetate | N/D | N/D | 6 | N/D | N/D | 6 |
| α-Ketoglutarate | 4.13838 | 0.75903 | 6 | 4.59542 | 1.20611 | 6 |
| Citrate | 13.62303 | 2.54623 | 6 | 18.90555 | 6.15514 | 6 |
| Fumarate | 18.45858 | 4.86432 | 6 | 18.25340 | 4.87964 | 6 |
| Glycolate | 15.98108 | 3.95365 | 6 | 22.06985 | 5.84236 | 6 |
| Isocitrate | 0.06610 | 0.02311 | 6 | 0.13932 | 0.05405 | 6 |
| Latate | 1766.93353 | 578.48516 | 6 | 1746.80590 | 601.60449 | 6 |
| Malate | 58.91552 | 14.13779 | 6 | 72.12150 | 15.94465 | 6 |
| Pyruvate | 30.74097 | 8.55534 | 6 | 36.28857 | 7.76691 | 6 |
| Succinate | 31.87855 | 7.40460 | 6 | 34.62477 | 11.90977 | 6 |
| | WT-IL-4 Treated | | | KO IL-4 Treated | | |
| | Mean | SD | N | Mean | SD | N |
| Glucose | 53.46483 | 42.37231 | 6 | 70.05433 | 24.89516 | 6 |
| AMP | 2.31558 | 0.52330 | 6 | 2.12143 | 0.49597 | 6 |
| ADP | 0.87580 | 0.25365 | 6 | 0.61712 | 0.11426 | 6 |
| ATP | 0.27355 | 0.05624 | 6 | 0.24000 | 0.04647 | 6 |
| GMP | 15.29183 | 3.24820 | 6 | 14.33038 | 3.31270 | 6 |
| GDP | 0.14788 | 0.03016 | 6 | 0.10978 | 0.01872 | 6 |
| GTP | 0.04348 | 0.00905 | 6 | 0.03030 | 0.00383 | 6 |
| UMP | 18.00397 | 3.19011 | 6 | 16.84180 | 4.16485 | 6 |
| UDP | 0.23513 | 0.05336 | 6 | 0.18030 | 0.02738 | 6 |
| UTP | 5.48045 | 1.23303 | 6 | 4.40850 | 0.91682 | 6 |
| cyclic-ADPR | 0.35188 | 0.05085 | 6 | 0.32667 | 0.05081 | 6 |
| cyclic-AMP | 0.00362 | 0.00065 | 6 | 0.00310 | 0.00050 | 6 |
| cyclic-GMP | N/D | N/D | 6 | N/D | N/D | 6 |
| cyclic-UMP | 0.00372 | 0.00205 | 6 | 0.00447 | 0.00164 | 6 |
| 6P-Gluconate | 0.01622 | 0.00197 | 6 | 0.02073 | 0.00781 | 6 |
| Acetoacetyl-CoA | N/D | N/D | 6 | N/D | N/D | 6 |
| Acetyl-CoA | 0.00738 | 0.00167 | 6 | 0.00588 | 0.00127 | 6 |
| Acetylglucosamine-1P | 0.13607 | 0.04500 | 6 | 0.10845 | 0.02050 | 6 |
| Acetylglucosamine-6P | 0.51317 | 0.17227 | 6 | 0.41278 | 0.09335 | 6 |
| Acetyl-Phosphate | 0.49598 | 0.10003 | 6 | 0.46855 | 0.10759 | 6 |
| ADP-Glucose | 0.00298 | 0.00069 | 6 | 0.00260 | 0.00069 | 6 |
| DHAP | 0.05342 | 0.01460 | 6 | 0.05052 | 0.01150 | 6 |
| Erythrose-4P | 0.05778 | 0.00918 | 6 | 0.03703 | 0.00709 | 6 |
| Fructose-1P | 0.00392 | 0.00188 | 6 | 0.00893 | 0.00761 | 6 |
| Fructose-6P | 0.06408 | 0.01990 | 6 | 0.05762 | 0.01736 | 6 |
| Fructose-1,6-bisP | 0.01960 | 0.01004 | 6 | 0.02422 | 0.01128 | 6 |
| Glucosamine-6P | 0.04035 | 0.00802 | 6 | 0.03932 | 0.00815 | 6 |
| Glucose-1P | 0.03848 | 0.01123 | 6 | 0.03615 | 0.01276 | 6 |
| Glucose-6P | 0.10443 | 0.02227 | 6 | 0.09843 | 0.02489 | 6 |
| Glucose-1,6-bisP | 0.04287 | 0.01103 | 6 | 0.04135 | 0.01323 | 6 |
| Glyceraldehyde-3P | 0.13652 | 0.04147 | 6 | 0.11277 | 0.02510 | 6 |
| Glycerate-2,3P | 0.00063 | 0.00012 | 6 | 0.00052 | 0.00023 | 6 |
| Glycerate-2P | 0.00803 | 0.00360 | 6 | 0.00902 | 0.00382 | 6 |
| Glycerate-3P | 0.06530 | 0.02847 | 6 | 0.07625 | 0.03384 | 6 |
| Glycerol-3P | 0.98198 | 0.22363 | 6 | 0.86308 | 0.21596 | 6 |
| Hs-CoA | 0.05170 | 0.01263 | 6 | 0.04430 | 0.00934 | 6 |
| Malonyl-CoA | N/D | N/D | 6 | N/D | N/D | 6 |
| Mannose-6P | 0.01458 | 0.00397 | 6 | 0.01255 | 0.00345 | 6 |
| NAD+ | 0.38838 | 0.08258 | 6 | 0.35507 | 0.08136 | 6 |
| NADH | 0.51585 | 0.12104 | 6 | 0.35150 | 0.05329 | 6 |
| NADP+ | 0.14310 | 0.03281 | 6 | 0.11395 | 0.02484 | 6 |
| NADPH | 0.11218 | 0.03541 | 6 | 0.17523 | 0.11594 | 6 |
| PEP | 0.03792 | 0.01627 | 6 | 0.04492 | 0.02136 | 6 |
| Phosphocreatine | 0.27473 | 0.04129 | 6 | 0.23377 | 0.06966 | 6 |
| Ribose-1,5bisP | 0.00238 | 0.00086 | 6 | 0.00195 | 0.00066 | 6 |
| Ribose-5P | 0.08472 | 0.02548 | 6 | 0.07803 | 0.01578 | 6 |
| Ribulose-5P | 0.26308 | 0.10146 | 6 | 0.18277 | 0.05131 | 6 |
| Ribulose-1,5bisP | 0.02488 | 0.00746 | 6 | 0.02412 | 0.00487 | 6 |
| Sedoheptulose-7P | 0.04902 | 0.01527 | 6 | 0.05303 | 0.01916 | 6 |
| Succinyl-CoA | 0.02933 | 0.00752 | 6 | 0.02340 | 0.00601 | 6 |
| UDP-acetylglucosamine | 1.09593 | 0.25603 | 6 | 0.93945 | 0.23716 | 6 |
| UDP-Glucose | 0.54668 | 0.10616 | 6 | 0.52665 | 0.12672 | 6 |
| Acetoacetate | N/D | N/D | 6 | N/D | N/D | 6 |
| α-Ketoglutarate | 3.78465 | 0.51476 | 6 | 3.98525 | 0.79468 | 6 |
| Citrate | 15.89095 | 1.17582 | 6 | 17.69467 | 6.25746 | 6 |
| Fumarate | 19.47978 | 4.31749 | 6 | 15.48413 | 2.95582 | 6 |
| Glycolate | 19.94110 | 2.78066 | 6 | 20.91850 | 4.66375 | 6 |
| Isocitrate | 0.11463 | 0.03969 | 6 | 0.20935 | 0.12834 | 6 |
| Latate | 2685.46775 | 806.81105 | 6 | 1872.38690 | 385.27969 | 6 |
| Malate | 87.06528 | 20.49614 | 6 | 73.43453 | 14.78257 | 6 |
| Pyruvate | 37.77848 | 7.98538 | 6 | 35.60545 | 5.54853 | 6 |
| Succinate | 52.06932 | 11.88115 | 6 | 36.14515 | 6.06897 | 6 |

### Lipin-1 Promotes Efferocytosis

Macrophages mediate tissue homeostasis by the clearance of dead and dying cells by a process termed efferocytosis. Engagement of AC elicits proresolving responses from macrophages. Recent studies have demonstrated that β-oxidation of AC-derived lipids is necessary for IL-10 production (proresolving cytokine) and subsequent wound healing in a mouse model of myocardial infarction. Defects in efferocytosis lead to unresolved inflammation that perpetuates disease pathogenesis, as seen in atherosclerosis, obesity, diabetes, and autoimmunity. The inventors have demonstrated that mice lacking myeloid-associated lipin-1 have increased necrotic cores within atherosclerotic plaques and have delayed wound closure (above). Inhibition of efferocytosis can contribute to increased necrotic core formation during atherosclerosis and delayed wound closure. The inventors re-examined data of the number of macrophages to the number of dead cells within wounds of lipin-1^{m}KO mice and littermate controls. Although there was no difference in the total number of dead cells in the wound between lipin-1^{m}KO mice and littermate controls, the inventors observed a greater ratio of macrophages/dead cells within wounds of lipin-1^{m}KO mice as compared with littermate controls (FIG. 13). When cell death is extensive, as seen in atherosclerotic plaques or within wounds, successive uptake of ACs by proresolving macrophages termed “continuing efferocytosis” is necessary. This may suggest that macrophages from lipin-1^{m}KO mice may have a defect in either efferocytosis or continuing efferocytosis.

The inventors decided to determine if lipin-1 is required for efferocytosis/continuing efferocytosis. To examine both primary efferocytosis (engulfment of a single AC) and continuing efferocytosis (engulfment of a second cell), the inventors performed an in vitro continuing efferocytosis experiment. The inventors selected incubation times based on previous work investigating continuing efferocytosis. Briefly, PKH26-labeled AC were added to WT and KO BMDMs at a 4:1 ratio and allowed to incubate for 2 h. Two hours allows the macrophages to engulf and begin to degrade AC-derived macromolecules from the primary efferocytic event. Cells were washed, and PKH67-labeled AC were added to the BMDMs at a 4:1 ratio and allowed to incubate for 45 min, which is sufficient time for engulfment of a second AC (continuing efferocytosis). Cells were then stained, and the percentage of PKH26⁺PKH67⁺ macrophages was quantified via flow cytometry. Although there was no difference in the ability to take in one AC (FIG. 14), there was a significant reduction in the uptake of multiple AC (continuing efferocytosis) in lipin-1-deficient macrophages compared with WT controls (FIG. 14). Although the difference in continuing efferocytosis between WT and KO BMDMs was modest, typically these modest differences are more exaggerated in vivo. To examine efferocytosis in vivo, the inventors subjected lipin-1^{mEnzy}KO, lipin-1^{m}KO, and littermate control mice to a zymosan model of inflammation. Briefly, mice were injected i.p. with 0.1 mg of zymosan. The inflammatory response was allowed to continue for 6 d to establish a proresolving milieu. Labeled AC were injected into the peritoneal cavity for 45 min. Peritoneal lavages were collected, and percentages of macrophages with labeled AC were quantified via flow cytometry. Lipin-1^{m}KO mice had a significant reduction in the percentage of macrophages with AC compared with both WT and lipin-1^{mEnzy}KO (FIGS. 15 and 16). Macrophages in the peritoneal cavity likely encounter AC prior to the addition of labeled AC, suggesting to the inventors a defect in secondary efferocytosis as observed in vitro. Lipin-1 transcriptional coregulatory function regulates PPARs, which have been shown to increase efferocytic receptors such as MerTK. A reduction in efferocytosis receptors in lipin-1 KO macrophages could explain the defect in efferocytosis. To determine if the loss of lipin-1 results in an alteration in cell surface expression of MerTK, the inventors stained peritoneal lavages for macrophages (CD11b and F4/80) and analyzed the presence of MerTK via flow cytometry. There was no significant difference in cell surface expression of MerTK in WT and lipin-1 KO macrophages (FIG. 45), suggesting the defect in efferocytosis is not due to the lack of MerTK. These data demonstrate that lipin-1 enzymatic activity is not involved in efferocytosis and suggest the transcriptional coregulatory activity promotes continuing efferocytosis in macrophages.

The inventors have shown that lipin-1 is required for increased OCR in response to free fatty acids, but the inventors wanted to know if lipin-1 regulates β-oxidation in response to AC during efferocytosis. BMDMs from WT, lipin-1^{mEnzy}KO, and lipin-1^{m}KO mice were either pretreated with or without etomoxir to prevent fatty acid transport into the mitochondria followed by the addition of AC to the macrophages at an AC/BMDM ratio of 4:1. AC were incubated with BMDMs for 30 min prior to the start of Seahorse analysis. This is sufficient time for differences in OCR due to (3-oxidation of AC-derived lipids to be observed. After 1 h of OCR readings, antimycin A and rotenone were added to the cells to inhibit electron import into the electron transport chain. This ensures that the observed oxygen consumption is specific to the mitochondria. OCR was then analyzed via Seahorse XFe analyzer (Agilent Technologies). WT and lipin-1 EKO macrophages significantly increased OCR in response to AC, with a significant reduction with pretreatment of etomoxir (FIGS. 17 and 18). This suggests that the increase in oxidative metabolism is due to the catabolism of AC-derived lipids in both WT and EKO macrophages. However, lipin-1 KO macrophages failed to respond to AC compared with WT macrophages, with no significant difference in OCR with pretreatment of etomoxir (FIGS. 19 and 20). Etomoxir treatment of unstimulated (no AC) WT, lipin-1 EKO, and lipin-1 KO macrophages allowed the inventors to determine basal lipid use. There was a significant reduction in OCR in WT, lipin-1 EKO, and lipin-1 KO macrophages with treatment with etomoxir alone (FIGS. 46 and 47). These data suggest that there is no defect in basal β-oxidation but an inability to undergo β-oxidation upon lipid loading in the inventors' lipin-1 KO macrophages. Together, these data imply that the transcriptional coregulatory function of lipin-1 is required for β-oxidation of AC-derived lipids during efferocytosis, whereas the enzymatic activity of lipin-1 is not required.

### Discussion

Using the inventors' lipin-1^{m}KO and lipin-1^{mEnzy}KO mice, the inventors demonstrated that lipin-1 enzymatic activity is dispensable for wound healing macrophage polarization and provided evidence suggesting that lipin-1 transcriptional co regulator function is required. Only macrophages lacking the entire lipin-1 protein failed to fully express canonical wound healing associated genes in response to IL-4. Furthermore, impaired healing of full excision wound was also observed in lipin-1^{m}KO mice but not lipin-1^{mEnzy}KO mice. There was no alteration in systemic myeloid immune composition of lipin-1^{m}KO mice after wounding, the inventors did observe increased macrophage content and these macrophages had a reduction in the wound healing associated marker CD206. Combined, these data suggest to the inventors that lipin-1 transcriptional co-regulator activity contributes to macrophage wound healing function that promotes wound closure.

Lipin-1 is a multi-functional protein having both enzymatic and transcriptional coregulator function. The removal of exons 3 and 4 of lipin-1 in the inventors' lipin-1^{mEnzY}KO mice results in truncated lipin-1 that lacks enzymatic activity but retains the ability of lipin-1 to bind to transcription factors such as PPARα and PPARγ. Removal of exon 7 from lipin-1 in the inventors' lipin-1^{m}KO mice causes a missense protein leading to loss of lipin-1 (and both activities). BMDMs from the lipin-1^{mEnzy}KO mice had equivalent expression of IL-4 elicited genes as WT BMDMs suggesting that lipin-1 enzymatic activity is dispensable for IL-4 mediated gene expression. Lipin-1^{m}KO BMDMs had reduced expression of IL-4 mediated wound healing genes. IL-4 binding to the IL-4R leads to phosphorylation and activation of STAT6 leading to macrophage wound healing polarization. STAT6 binds to DNA promoters that leads to recruitment of PPARγ:RXR transcription factors to promote gene expression in macrophages. In adipocytes and hepatocytes, lipin-1 binds to and augments the activity of both PPARγ and PPARγ. In addition to augmentation of PPARγ and PPARγ activity, lipin-1 inhibits SREBP and NFAT by displacing them from their native promoters. The inventors propose lipin-1 transcriptional co-regulator activity promotes macrophages to a wound healing state during IL-4 stimulation. In support of this, macrophages lacking PPARγ fail to polarize to a wound healing phenotype, similar to the phenotype the inventors observed in macrophage lacking full lipin-1. SREBP activity promotes the activation of the NLRP3 inflammasome leading to pro-inflammatory responses. While not typically active in macrophages, continued stimulation of macrophages lead to NFAT activity and promotion of pro-inflammatory responses such as IL-6 and TNF-α. IL-6 and TNF-α are known to inhibit wound healing polarization. Thus, lipin-1 may also be repressing the activity of NFAT and SREBP allowing wound healing polarization. The inventors' results and these published observations suggest to the inventors that lipin-1 transcriptional coregulator activity promotes wound healing polarization.

Macrophage polarization is critical for effective in vivo wound healing where the number and phenotype of the resident and recruited macrophages determine the extent and efficiency of healing. Up to 1 day after wounding, pro-inflammatory macrophages initiate an acute inflammatory response; after that time frame, wound healing macrophages promote angiogenesis and tissue formation. The loss of enzymatic activity of lipin-1 reduces pro-inflammatory macrophage polarization. The inventors observed no defect in wound closure in lipin-1^{mEnzy}KO mice compared to litter mate controls demonstrating that lipin-1 enzymatic activity is dispensable for myeloid-mediated wound closure. In contrast, mice lacking both lipin-1 activities had a defect in wound closure. The inventors propose that the lipin-1 transcriptional co-regulatory activity in myeloid cells is responsible for aiding in wound closure during a full excisional wound. Mice lacking PPARγ from myeloid cells (LysMCre model) exhibit a significant delay in wound healing due to compromised granulation, collagen deposition, angiogenesis and a failure in clearance of apoptotic cells. PPARγ activation also promotes macrophage associated CD206 expression during a mouse model of liver wounding, providing evidence that PPAR activity promotes CD206 expression on macrophages during wounding. The inventors see a significant reduction in CD206 gene expression in lipin-1^{m}KO BMDMs and surface expression on macrophages isolated from the wounds of lipin-1^{m}KO mice. Myeloid associated SREBP activity also contributes to wound closure, as mice with loss of SREBP activity in myeloid cells (LysMCre model) had enhanced wound closure. Lipin-1 can inhibit the activity of SREBP. Taking together the inventors propose that the lipin-1 transcriptional co-regulatory activity in myeloid cells promotes beneficial wound closure responses.

The inventors propose that the loss of wound healing observed in the inventors' lipin-1^{m}KO mice is due to loss of transcriptional co-regulator activity from monocytes and macrophages. However, LysM-Cre was used to knockout lipin-1 in the inventors' mice, and LysM expression is not restricted to monocytes and macrophages. Analysis of LysM-Cre-mediated gene deletion demonstrates gene excision in dendritic cells (DC) and neutrophils, as well as monocytes and macrophages. The inventors suggest that loss of lipin-1 in DC is not responsible for the difference in wound closure in the inventors' lipin-1^{m}KO mice. DCs enhance T cell/B cell responses, rather than innate immune responses, and the inventors' difference in wound closure is more prevalent in earlier phase of healing (likely prior to T cell responses). The contribution of lipin-1 to DC function is completely unknown and needs to be looked at in the future. Neutrophils clearly contribute to wound healing and the inventors observe PMNs within the wounds of both WT and Lipin-1^{m}KO mice. Lipin-1 is not readily detected in neutrophils, however, if inflammation drive increases in lipin-1 expression in neutrophils is unknown. Future work will need to address the possibility of neutrophil-associated lipin-1 contribution to wound closure. The inventors propose though, that the most likely effect of myeloid loss of lipin-1 is on macrophage function. The inventors observe reduction in wound healing associated gene in lipin-1^{m}KO BMDMS that are known to contribute to wound closure, and a reduction in CD206 surface expression on macrophages within the wounds of lipin-^{m}KO mice. Thus, though possible loss of lipin-1 in other cells beside macrophages may contribute to reduction in wound closure, at the very least the loss of lipin-1 in macrophages is also a contributing factor.

The inventors' data highlight the role of lipin-1 transcriptional co-regulator activity within macrophage function, specifically for wound healing polarization. Furthermore, the inventors provide evidence that the lack of myeloid-associated lipin-1 transcriptional co-regulator activity has in vivo consequences. Macrophage responses are now recognized to play crucial roles in a diverse array of pathologies like atherosclerosis, arthritis, osteoporosis, and sterile inflammation. Beyond sterile inflammation, IL-4 mediated macrophage responses are critical to control and clearance of numerous parasitic infections as well. Thus, the contribution of lipin-1 to myeloid cells function is likely to be important beyond sterile inflammation. Future work will be needed to better understand the mechanisms by which lipin-1 transcriptional co-regulator activity drives macrophage function in different pathological conditions of sterile inflammation and parasitic infections.

### Further Embodiment

### Discussion

Proresolving macrophages undergo drastic metabolic changes to effectively respond to stimuli. These proresolving macrophages significantly increase β-oxidation and oxidative phosphorylation, and perturbations in these pathways are deleterious to macrophage function. The inventors have previously demonstrated that lipin-1 contributes to atherosclerotic progression and macrophage polarization toward a proresolving phenotype in response to IL-4. Using the inventors' lipin-1^{mEnzy}KO, lipin-1^{m}KO, and littermate control mice, the inventors provide evidence that lipin-1 transcriptional coregulator function is critical for induction of β-oxidation in response to proresolving stimuli. The inventors also provide evidence that the loss of lipin-1 transcriptional coregulatory activity also impacts efferocytic function of macrophages, which may contribute to impairment in disease resolution. These results are some of the first (to the inventors' knowledge) to implicate lipin-1 activity in regulating continuing efferocytosis.

IL-4 stimulation, as well as efferocytosis, leads to activation of transcription factors such as STATE and PPARs to drive expression of genes involved in fatty acid transport and β-oxidation. Lipin-1 binds to and augments PPAR activity in a variety of tissues. The removal of exons 3 and 4 of lipin-1 in the inventors' lipin-1^{mEnzy}KO mice results in truncated lipin-1 that lacks enzymatic activity but retains the ability of lipin-1 to bind to transcription factors such as PPARα and PPARγ. Lipin-1^{mEnzy}KO macrophages had equivalent levels of OCR in response to both IL-4 stimulation and AC. Removal of exon 7 from lipin-1 in the inventors' lipin-1^{m}KO mice causes a missense protein, leading to loss of lipin-1 and both activities. BMDMs from lipin-1^{m}KO mice showed a significant reduction in OCR in response to IL-4 and ACs. Loss of lipin-1 did not result in reduction of either free lipid or AC, suggesting that the defect in β-oxidation is not due to the inability of the macrophages to take in the lipid or perform primary efferocytosis. Further studies will investigate trafficking and use of the lipid within the macrophage after either free fatty acid feeding or AC feeding.

Macrophage metabolism is directly connected to their polarization state and role during the inflammatory response. Central carbon analysis of WT and lipin-1 KO macrophages untreated and treated with IL-4 revealed an overall increase in glycolysis in lipin-1-deficient macrophages. Interestingly, the loss of lipin-1 led to a significant decrease in lactate, with no alteration in pyruvate. Lactate can be shuttled into the mitochondria to be converted to pyruvate to fuel the TCA cycle. Untreated and IL-4-treated lipin-1 KO macrophages have an accumulation of isocitrate, an isomer of citrate. Downstream TCA cycle metabolites after isocitrate were reduced in lipin-1 KO macrophages compared with WT macrophages. This is further supported by a decrease in NADH levels as well as a significant decrease in OCR in lipin-1-deficient macrophages. Isocitrate is an isomer of citrate, which can be exported out of the mitochondria to be used in lipid synthetic pathways, similar to proinflammatory macrophages. Lipin-1-deficient macrophages have an increase in the initial metabolites of the PPP, resulting in increased NADPH levels. NADPH is used by the enzymes of lipid synthetic pathways as well as for redox potential within the cell. Glycerol-3-phosphate is used as a backbone for glycerol lipid synthesis and is elevated in the lipin-1 KO macrophages (table above). Altogether, the loss of lipin-1 results in a metabolic profile that is supportive of lipid synthesis, rather than lipid catabolism. Interestingly, previous studies have shown that de novo lipid synthesis, such as ceramides, is detrimental to efferocytosis, supporting data shown in the inventors' current study. The inability to undergo β-oxidation of exogenous lipid, together with the metabolic profile of lipin-1 KO macrophages, may suggest that the incoming free fatty acids are being used for de novo lipid synthesis.

Efferocytosis is a complex process that leads to the accumulation of macromolecules within the macrophage that need to be processed and cleared. AC-derived arginine is metabolized, leading to RAC1 activation and further actin polymerization to allow for uptake of multiple AC). Lipid is a major component of AC. Mitochondria are critical for the process of efferocytosis. Mitochondria must undergo fission to mobilize around the engulfed AC to allow for continuing efferocytosis. This is presumably to accept incoming fatty acids for β-oxidation. β-Oxidation of AC-derived lipids is required for anti-inflammatory cytokine production and wound healing in a mouse model of myocardial infarction. These studies eloquently show that β-oxidation contributes to functional outcomes of efferocytosis during disease resolution. The inventors demonstrated that loss of lipin-1 did not impact primary efferocytosis but rather secondary or continuing efferocytosis. Furthermore, the inventors demonstrate a lack of increased β-oxidation in response to AC stimulation. The inventors speculate that during primary efferocytosis, β-oxidation is not required for engulfment of AC. The inventors' work with zymosan particles supports this idea as baseline engulfment of these particles is not impacted by loss of lipin-1 in macrophages, but there is a reduction in zymosan particle uptake during IL-4-mediated enhancement. The inventors believe that following engulfment of the first AC, there is an abundance of lipid that must be processed and degraded via β-oxidation that is mediated by lipin-1 before the macrophage is able to take in another AC (continuing efferocytosis). Currently the mechanism connecting β-oxidation is unknown, but there are several potential hypotheses. There may be a limit on the amount of engulfed lipid that macrophage can handle, which must be cleared prior to accepting a second AC. The inventors feel this is unlikely based on the amount of lipid macrophage foam cells engulf. There is the potential that β-oxidation supplies energy more efficiently than other mechanisms, allowing for continuing efferocytosis. There may be downstream metabolites generated from the degradation of AC-derived lipid that mediate signaling cascades to promote continuing efferocytosis, similar to Arginine-mediated enhancement of continuing efferocytosis.

The inventors' data suggest that not only is the transcriptional coregulatory activity of lipin-1 required for increased oxidative phosphorylation and lipid catabolism in macrophages in response to proresolving stimuli, but it is required for functional outcomes of that stimulation as well. The inventors show that lipin-1 is required for efficient continuing efferocytosis. Mice lacking myeloid-associated lipin-1 have reduced AC uptake in a zymosan model of efferocytosis. Furthermore, the inventors' data above demonstrate that the loss of lipin-1 results in a reduction in proresolving/wound-healing polarization in response to IL-4 and defects in wound healing in vivo. The inventors would propose that lipin-1 transcriptional coregulatory activity is critical to macrophage proresolving polarization. Further studies will investigate how lipin-1 transcriptional coregulatory activity primes β-oxidation in macrophages, if β-oxidation is required for macrophages to perform continuing efferocytosis, and how β-oxidation allows for continuing efferocytosis.

### Wound Healing

Traditional hemostatic materials include tourniquets, bandages and sterilized dressings. Other hemostatic materials include fibrin glues (FG), oxidized celluloses (OC), oxidized regenerated celluloses (ORC) and mineral zeolite-based hemostats.

Fibrin glues are hemostatic adhesives that are biocompatible, and which likely mimic the spontaneous coagulation process while being independent of platelets and coagulation factors. Commercially available fibrin-based glue products include Beriplast P, Hemaseel, Biocol, Boheal and Quixil, etc. However, fibrin glues are costly to produce, may be a source of blood-borne diseases and infections, are complicated to apply and are slow in arresting bleeding.

Oxidized celluloses and oxidized regenerated celluloses are degradable, have antibacterial and hemostatic properties, and are especially effective in arresting slow bleeding. The hemostatic mechanism with these materials is proposed to be that the acidic carboxyl group in the molecule binds with the Fe³⁺ ion in the hemoglobin to generate the acidic Fe³⁺-hemin in blood, whereby red-brown gel blocks are formed to close the end of capillaries thereby arresting the bleeding. Nevertheless, the oxidized celluloses and oxidized regenerated celluloses may expand, which in turn may cause neurothlipsis. Examples of commercially available OC and ORC hemostats include the Oxycel series and the Surgical series.

Inert mineral zeolite particles were first found to have a hemostatic effect in the 1980s (see U.S. Pat. No. 4,822,349). In 2002, Z-Medica Corporation produced a type of new hemostatic material under the name of QuikClot™. These zeolite-based materials are apparently superior to other hemostatic materials in hemostatic efficacy. The hemostatic mechanism of mineral zeolites mainly resides in their extraordinary selective adsorption of water relative to erythrocytes, platelets and other coagulation factors, which leads to a quick hemostasis by concentrating the clotting factors at the injury site. However, mineral zeolite hemostats may be recognized as “foreign” and are not biodegradable.

The invention provides methods and compositions to reduce or arrest the flow of blood from a wound, e.g., by accelerating would healing, and optionally preventing or inhibiting microbial infection in the wound area, or any combination thereof. In one embodiment, the invention provides a substrate, e.g., a protective covering for a wound comprising a composition of the invention. The compositions of the invention promote and accelerate healing as a result of the presence of a combination of agents. In one embodiment, the composition includes an absorbent agent, such as aluminum silicate, and a blood vessel constricting agent, such as aluminum sulfate or caffeine, and a macrophage proresolving polarizer such as lipin-1. The composition may also include a blood clotting agent, e.g. fibrin, in an effective amount. In another embodiment, the composition includes a blood vessel constricting agent, e.g., aluminum sulfate or caffeine, and a blood clotting agent in an effective amount. In one embodiment, the invention provides an anhydrous mixture, e.g., in powder form, comprising a combination of agents forming a composition of the invention. Compositions of the invention can be hydrated in the presence of blood, wound exudate, or other selected liquid or aqueous media which promotes clotting of the blood. In one embodiment, the invention provides a gel comprising a composition of the invention. In one embodiment, the invention provides an aerosol comprising a composition of the invention.

In one embodiment, the invention provides a support, e.g., a bandage or other wound dressing, that includes a macrophage proresolving polarizer (e.g., lipin-1), an absorbent agent, a blood vessel constricting agent, and optionally an antiseptic agent such as an antimicrobial agent, optionally a topical analgesic or anesthetic agent, optionally isolated fibrin or components that yield fibrin, e.g., isolated fibrinogen and isolated thrombin, or any combination of optional component(s). In one embodiment, the invention provides a support that includes a blood vessel constricting agent and isolated fibrin or components that yield fibrin, e.g., isolated fibrinogen and isolated thrombin, and optionally an antiseptic agent such as an antimicrobial agent, and optionally a topical analgesic or anesthetic agent.

Thus, in one embodiment the invention provides a composition for wound healing comprising an amount of a macrophage proresolving polarizer (e.g., lipin-1) and may also include IL-4 and apoptotic cell (AC) derived lipids. The composition may also include aluminum sulfate. The composition may also include an amount of a blood clotting agent. In one embodiment, the composition is in powder form. In one embodiment, the composition is in aerosol form. In one embodiment, the composition is in gel form. In one embodiment, the composition is applied to a support, e.g., a bandage. The composition may further include a local anesthetic agent and/or an antiseptic such as an antimicrobial agent.

In another embodiment, the invention provides a composition for wound healing comprising an amount a macrophage proresolving polarizer (e.g., lipin-1) and may also include IL-4 and apoptotic cell (AC) derived lipids and an amount of fibrin or an amount of fibrinogen and thrombin. The composition may also include aluminum sulfate. In one embodiment, the composition is in powder form. In one embodiment, the composition is in aerosol form. In one embodiment, the composition is in gel form. In one embodiment, the composition is applied to a support, e.g., a bandage. The composition may further include a local anesthetic agent and/or an antiseptic such as an antimicrobial agent.

In another embodiment, the invention provides a composition for wound healing comprising an amount of a macrophage proresolving polarizer (e.g., lipin-1). The composition may be in any form which may effectively deliver a macrophage proresolving polarizer (e.g., lipin-1) to a wound, such as a powder form, an aerosol form, or a gel form. In one embodiment, the composition is applied to a support, e.g., a bandage. The composition may further include a blood vessel constricting agent, such as aluminum sulfate, a blood clotting agent, such as fibrin, a local anesthetic agent, and/or an antiseptic such as an antimicrobial agent.

In one embodiment, a composition of the invention comprises a gel comprising an absorbent agent and a blood vessel constricting agent, and optionally an antiseptic agent such as an antimicrobial agent, optionally a topical analgesic or anesthetic agent, optionally isolated fibrin or components that yield fibrin, e.g., isolated fibrinogen and isolated thrombin, or any combination of optional components. In one embodiment, a composition of the invention comprises a gel comprising a blood constricting agent and isolated fibrin or components that yield fibrin, e.g., isolated fibrinogen and isolated thrombin, and optionally an antiseptic agent such as an antimicrobial agent, and optionally a topical analgesic or anesthetic agent.

In one embodiment, a composition of the invention comprises an aqueous liquid comprising an a macrophage proresolving polarizer (e.g., lipin-1), and an absorbent agent and a blood vessel constricting agent, and optionally an antiseptic agent such as an antimicrobial agent, optionally a topical analgesic or anesthetic agent, optionally isolated fibrin or components that yield fibrin, e.g., isolated fibrinogen and isolated thrombin, or any combination of optional component(s). In another embodiment, a composition of the invention comprises an aqueous liquid comprising macrophage proresolving polarizer (e.g., lipin-1), a blood vessel constricting agent and isolated fibrin or components that yield fibrin, e.g., isolated fibrinogen and isolated thrombin, and optionally an antiseptic agent such as an antimicrobial agent, and optionally a topical analgesic or anesthetic agent.

In one embodiment, the composition of the invention is a solid, e.g., a powder form. In another embodiment, the composition of the invention comprises an aerosol or spray having a macrophage proresolving polarizer (e.g., lipin-1), optionally an absorbent agent and a blood vessel constricting agent, and optionally an antiseptic agent such as an antimicrobial agent, optionally a topical analgesic or anesthetic agent, optionally isolated fibrin or components that yield fibrin, e.g., isolated fibrinogen and isolated thrombin, or a blood vessel constricting agent and isolated fibrin or components that yield fibrin, e.g., isolated fibrinogen and isolated thrombin, and optionally an antiseptic agent such as an antimicrobial agent, and optionally a topical analgesic or anesthetic agent. In one embodiment, a substantially anhydrous composition of the invention is provided. A composition of the invention may be applied as a powder, a liquid, an aerosol or spray, or a dry support, e.g., dressing, having a composition of the invention applied thereto and/or embedded therein.

Also provided are methods of making and using the compositions and supports having the compositions of the invention.

Embodiments of the disclosed invention relate to devices, e.g., supports such as bandages, and compositions, e.g., gels, aqueous liquids, powders and sprays, to facilitate the reduction in bleeding, to initiate and provide for hemostasis, and/or to promote wound healing, and/or methods of making and using the devices and compositions.

In one embodiment, the invention provides a support, e.g., a bandage or other wound dressing comprising an amount of a macrophage proresolving polarizer (e.g., lipin-1), and optionally a blood vessel constricting agent and an amount of isolated fibrin or components that yield fibrin, e.g., isolated fibrinogen and isolated thrombin, optionally an adsorbent or absorbent agent, optionally an antiseptic agent such as an antimicrobial agent, optionally a topical analgesic or anesthetic agent, or any combination of optional components. In another embodiment, the invention provides a support comprising an amount of macrophage proresolving polarizer (e.g., lipin-1), optionally a blood vessel constricting agent and an amount of an absorbent agent, optionally an amount of isolated fibrin or components that yield fibrin, optionally an antiseptic agent, optionally a topical analgesic or anesthetic agent, or any combination of optional components. A “wound dressing” includes any pharmaceutically acceptable wound covering or support matrix such as, for example, a film, including those of a semipermeable or a semi-occlusive nature such as polyurethane copolymers, acrylamides, acrylates, paraffin, polysaccharides, cellophane and lanolin, and hydrocolloids including carboxymethylcellulose, protein constituents of gelatin, pectin, and complex polysaccharides including Acacia gum, guar gum and karaya. These materials may be utilized in the form of a flexible foam or, in the alternative, formulated in polyurethane or, in a further alternative, formulated as an adhesive mass such as polyisobutylene; starch or propylene glycol; which typically contain about 80% to about 90% water and are conventionally formulated as sheets, powders, pastes and gels in conjunction with cross-linked polymers such as polyethylene oxide, polyvinyl pyrollidone, acrylamide, propylene glycol; a foam such as polysaccharide which consist of a hydrophilic open-celled contact surface and hydrophobic closed-cell polyurethane, and other materials including pine mesh gauze, paraffin and lanolin-coated gauze, polyethylene glycol-coated gauze, knitted viscose, rayon, and polyester and cellulose-like polysaccharide such as alginates, including calcium alginate, which may be formulated as non-woven composites of fibers or spun into woven composites.

In one embodiment, the invention provides a device for promoting wound healing, and preferably the clotting of blood, thereby controlling bleeding and infection. The device comprises a gauze substrate, e.g., cotton cellulose formed as woven or non-woven gauze, and a composition of the invention disposed on the gauze substrate. Upon the application of the device to the bleeding wound, at least a portion of the components of the composition comes into contact with the blood to cause a macrophage proresolving polarizing effect and a hemostatic effect. In another embodiment, the bandage has a flexible substrate and a gauze substrate mounted thereon.

According to another aspect, the invention provides a wound dressing, such as a bandage that can be applied to a bleeding wound to promote wound healing and the clotting of blood, thereby controlling bleeding and infection. In one embodiment, the bandage comprises a substrate, a mesh mounted on the substrate, and a composition of the invention retained in the mesh. The mesh has a plurality of members arranged to define openings that allow for the flow of blood into the mesh and into the composition of the invention, thereby producing a clotting effect.

In one embodiment, a patch bandage comprises an absorbent fiber pad which is backed up by and located at the center of a holding strip, the pad having a composition of the invention applied to the surface and/or embedded therein and the strip having an adhesive surface with the pad being affiliated to the surface. The bandage may be placed over a cut or wound to cover the wound with the pad covering the wound to permit the pad to absorb the blood flow therefrom and permit the adhesive surface of the strip to adhere to the skin and hold the bandage in place. The bandage may be provided in a closed sterile receptacle or container or the like.

In one embodiment, the invention provides a wound healing and hemostatic sponge that can be applied to a bleeding wound to promote healing, clot blood and control bleeding. Such a sponge comprises a substrate, and a macrophage proresolving polarizer (e.g., lipin-1) composition, of the invention disposed on a first surface of the substrate or dispersed in the substrate. Another type of sponge has first and second substrates. A macrophage proresolving polarizer (e.g., lipin-1) composition, of the invention is applied to the first substrate, and the second substrate is placed on the macrophage proresolving polarizer (e.g., lipin-1) composition. When this sponge is used to treat a bleeding wound, applying the sponge causes at least a portion of the macrophage proresolving polarizer material, and optionally hemostatic material, to come into contact with blood through at least one of the substrates. The sponge may comprise a film and a macrophage proresolving polarizer of the invention incorporated into the film; a substrate, a macrophage proresolving polarizer of the invention disposed on the substrate, and a film disposed over the macrophage proresolving polarizer composition of the invention; or a macrophage proresolving polarizer composition of the invention sandwiched between two substrates.

Thus, the composition can be used in solid form (e.g., retained in a mesh or in a film), or it can be used in powder form (e.g., deposited on a fibrous substrate to form a gauze or a sponge).

In one embodiment, the combination of agents disclosed herein greatly enhances wound healing, inhibits microbial infection, and/or accelerates blood clotting, and may be combined with a covering or carrier (support) such as a bandage, cotton gauze and the like. In one embodiment, a wound treated with a composition of the invention is subsequently covered with a suitable wound covering or dressing. In another embodiment, a wound covering or dressing is impregnated or coated with a dry powder form of a composition of the invention and applied to the wound. Thus, the present invention can also be practiced in conjunction with wound coverings, dressings, and protective materials, such as bandages, cotton gauze, and the like.

The invention also concerns kits comprising in one or more containers or packages having a plurality of components for a composition of the invention. In one embodiment, a composition of the invention is packaged in a container that is designed in a manner so as to preserve the anhydrous nature of the composition until the container is opened. A kit of the invention can also comprise a container having a quantity of suitable powder or spray liquid or aqueous media, for application to a wound. In one embodiment, the powder spray or liquid or aqueous media is provided in sterile form. A kit of the present invention can also comprise a wound covering, dressing, or other wound or surgical site protective material, e.g., one maintained in sterile form until the package or container is opened for use.

In one embodiment, a kit comprises a dressing that includes a pad that contains a composition of the invention within and/or on the surface of the pad. In one embodiment, the pad is composed of porous foam that is sufficiently open to allow a free flow of powder to fill the voids in the porous foam. The open voids can either be random (like a foam air conditioning filter) or organized into tunnels. The tunnels can keep compositions from mixing until needed. The tunnels can be round holes or geometric shapes. Around the perimeter of the randomly open foam a less porous border may be used to contain the composition. The pad can be designed so that lateral pressure can compress the foam or tunnels and hold the composition in place for inverted application.

In another embodiment, a kit comprises a dressing having a pad with fibers perpendicularly oriented to the plane of the pad, wherein the fibers can hold and release a composition of the present invention. The dressing can be provided with or without an integrated foam or fabric or substrate backing. The dressing can be pre-loaded with a composition of the present invention. The dressing can be of a design wherein the fibers remain attached to the dressing during and/or after application to a wound or surgical site.

In one embodiment, a kit comprises a wound dressing with a flocked pad wherein the pad has a foam (e.g., polyurethane) portion and a flocked fibers portion. In one embodiment, the foam portion is a porous foam as described above. In this embodiment, a composition of the invention can be loaded onto the side of the foam opposite that of the fibers and the composition could then travel or flow through the foam and onto the fibers. The fibers can be attached to the foam portion and can be made, for example, out of calcium alginate. The fibers can be a woven or non-woven material. The fibers can be composed of any suitable material such as cotton, wool, etc. In one embodiment, the fibers are composed of a velvet fabric. The fibers can be coated or flocked with a composition of the present invention. Optionally, the fibers can be composed of dissolvable material (e.g., polyvinyl alcohol) or a biodegradable material (e.g., starch, calcium alginate, polysaccharides, etc.). In one embodiment, the fibers can be composed of a material that can dissolve in a solution, such as a saline solution. In another embodiment, the fibers themselves do not dissolve in solution but are attached to the pad portion via a substance or material that itself can dissolve in solution. This permits a solution to be contacted with a dressing that has been applied to a site where blood has coagulated and formed a scab, wherein the fibers dissolve or the attachment dissolves and the pad portion of the dressing can then be easily removed without ripping the scab off the wound.

Compositions of the invention may be stored under substantially anhydrous conditions. Compositions of the invention can be provided in a sterile form.

Compositions of the subject invention can also comprise additional optional compounds or agents that provide for anti-microbial, analgesic or anesthetic, increased absorptive, and/or increased wound healing properties.

The dosage or amount of the components of a composition of the invention to be typically administered can be readily determined and will be dependent on various factors, such as the size and type of wound, the amount of blood or fluid present in the wound, and physical characteristics of the patient, as well as other drugs or treatments the patient is receiving.

The compositions of the invention are easy to use and to apply to a wound, and absorb wound exudates (which reduces odors and microbial action at the wound site) and so may also be used to treat lesions, trauma, injuries, incisions, and/or burns wherein stopping or slowing the flow of blood from a wound, incision, or medical treatment site is indicated.

Following application of a composition of the invention, e.g., a spray or gel, the wound may be left exposed to the air, or the wound may optionally be covered with a bandage or other suitable wound covering, e.g., one that includes a composition of the invention.

Exemplary Compositions

Disclosed herein are wound healing and optionally hemostatic devices and wound healing and optionally hemostatic agents that are applicable to would to promote healing and to bleeding wounds to promote hemostasis. In one embodiment, the wound healing agents generally include a macrophage proresolving polarizer such as lipin-1 that promote macrophage proresolving. In one embodiment hemostatic agents generally include absorbent agents such as silica-based materials that, when brought into contact with a bleeding wound, can minimize or stop blood flow by absorbing at least portions of the liquid phases of the blood, thereby facilitating clotting. In one embodiment, the absorbent agent of the invention is mixed with or otherwise used in conjunction with other materials to provide additional clotting functions and/or improved efficacy, including a blood vessel constricting agent, and optionally agents that provide an antiseptic environment at the wound site or to provide functions that are supplemental to the clotting functions, including, but are not limited to, pharmaceutically-active compositions such as antibiotics, antifungal agents, antimicrobial agents, anti-inflammatory agents, analgesics, antihistamines (e.g., cimetidine, chloropheniramine maleate, diphenhydramine hydrochloride, and promethazine hydrochloride), compounds containing silver or copper ions, combinations of the foregoing, and the like. Other materials that can be incorporated to provide additional hemostatic functions include ascorbic acid, tranexamic acid, rutin, and thrombin. Botanical agents having desirable effects on the wound site may also be added.

In one embodiment, the substrate is an absorbent gauze material that defines a matrix. Other materials from which the substrate may be fabricated include woven fabric, non-woven fabric, paper (e.g., kraft paper and the like), and cellulose material (e.g., cotton in the forms of balls, swabs, and the like), as well as other materials such as rayon/polyester cellulose blends and the like are also within the scope of the present invention.

In one embodiment, a composition of the present invention is woven into the fibers of the substrate. For example, a composition which includes an effective amount of a macrophage proresolving polarizer (e.g., lipin-1), and which optionally includes aluminum sulfate and other ingredients such as fibrin, a local anesthetic agent, and/or an antiseptic agent, may be woven into the fibers of an absorbent tissue/paper product. The product may then be used in wound cleaning and to blot up blood resulting from minor cuts or scrapes. It may also be used in a bandage or other wound dressing to facilitate wound healing, to promote blood clotting, to provide an anesthetic effect, and/or to provide an antiseptic effect.

In one embodiment, the invention provides a composition which includes an effective amount of a macrophage proresolving polarizer (e.g. lipin-1) optionally having isolated fibrin, or isolated fibrinogen and isolated thrombin, and also optionally aluminum sulfate and/or a local anesthetic agent and/or an antiseptic agent. In one embodiment, a composition of the invention includes a macrophage proresolving polarizer (e.g. lipin-1) and an absorbent agent such as aluminum sulfate and a blood vessel constricting agent (e.g., aluminum sulfate or caffeine). In one embodiment, a composition of the invention includes a macrophage proresolving polarizer (e.g. lipin-1) and an absorbent agent such as aluminum sulfate, a blood vessel constricting agent (e.g., aluminum sulfate or caffeine), and a local (e.g., topical) anesthetic agent. In one embodiment, a composition of the invention includes a macrophage proresolving polarizer (e.g. lipin-1) and an absorbent agent such as aluminum sulfate, a blood vessel constricting agent (e.g., aluminum sulfate or caffeine), a local anesthetic agent and an antiseptic agent. In one embodiment, a composition of the invention includes a macrophage proresolving polarizer (e.g. lipin-1) and an absorbent agent such as aluminum sulfate, a blood vessel constricting agent (e.g., aluminum sulfate or caffeine), isolated fibrinogin and isolated thrombin (or isolated fibrin). In one embodiment, a composition of the invention includes a macrophage proresolving polarizer (e.g. lipin-1) and an absorbent agent such as aluminum sulfate, a blood vessel constricting agent (e.g., aluminum sulfate or caffeine), isolated fibrinogin and isolated thrombin (or isolated fibrin), and a local anesthetic agent. In one embodiment, a composition of the invention includes a macrophage proresolving polarizer (e.g. lipin-1) and an absorbent agent such as aluminum sulfate, a blood vessel constricting agent (e.g., aluminum sulfate or caffeine), isolated fibrinogin and isolated thrombin (or isolated fibrin), a local anesthetic agent and an antiseptic agent. In one embodiment, the invention provides a bandage, gel or spray having a composition of the invention.

In one embodiment, a composition of the invention has a macrophage proresolving polarizer (e.g. lipin-1) and a local (e.g., topical) anesthetic agent. In one embodiment, a composition of the invention has a blood vessel constricting agent (e.g., aluminum sulfate or caffeine), a local anesthetic agent and an antiseptic agent. In one embodiment, a composition of the invention has a blood vessel constricting agent (e.g., aluminum sulfate or caffeine), isolated fibrinogin and isolated thrombin (or isolated fibrin). In one embodiment, a composition of the invention has a blood vessel constricting agent (e.g., aluminum sulfate or caffeine), isolated fibrinogin and isolated thrombin (or isolated fibrin), and a local anesthetic agent. In one embodiment, a composition of the invention has a blood vessel constricting agent (e.g., aluminum sulfate or caffeine), isolated fibrinogin and isolated thrombin (or isolated fibrin), a local anesthetic agent and an antiseptic. In one embodiment, the invention provides a bandage, gel or spray having a composition of the invention.

In one embodiment, a macrophage proresolving polarizer (e.g. lipin-1) either an absorbent agent ore a blood vessel constricting agent (e.g., aluminum sulfate and/or caffeine) are in a ratio of 0.01:1 to 1:1 by volume. In one embodiment, a macrophage proresolving polarizer (e.g. lipin-1), an absorbent agent, a blood vessel constricting agent (e.g., aluminum sulfate and/or caffeine), plus a local anesthetic agent such as lidocaine, tetracaine, or benzocaine or prilocaine, are employed in a composition, e.g., one in which the formulation ranges from 0.01:1 to 1:1 a macrophage proresolving polarizer (e.g. lipin-1) to absorbent agent or blood vessel constricting agent, with an effective amount of a local anesthetic agent. In one embodiment, a macrophage proresolving polarizer (e.g. lipin-1), an absorbent agent, a blood vessel constricting agent (e.g., aluminum sulfate and/or caffeine), a local anesthetic agent such as lidocaine, tetracaine, or benzocaine, and an antimicrobial, such as neomycin sulfate, polymyxin B, bacitracin zinc, pramoxine or prilocaine, are employed in a composition of the invention. In one embodiment, a macrophage proresolving polarizer (e.g. lipin-1), an absorbent agent and/or a blood vessel constricting agent (e.g., aluminum sulfate and/or caffeine), and isolated fibrinogin and isolated thrombin (or isolated fibrin) are employed in a composition, e.g., one in which the formulation ranges from 0.0001:1 to 1:1 macrophage proresolving polarizer (e.g. lipin-1) to absorbent agent and/or blood vessel constricting agent, with an effective amount of fibrin. In one embodiment, a macrophage proresolving polarizer (e.g. lipin-1), an absorbent agent and/or a blood vessel constricting agent, isolated fibrinogin and thrombin (or isolated fibrin), and a local anesthetic agent such as lidocaine, tetracaine, or benzocaine or prilocaine, are employed, in which the formulation ranges from 0.0005:1 to 1:1 macrophage proresolving polarizer (e.g. lipin-1) to absorbent agent and/or blood vessel constricting agent, with effective amounts of fibrin and a local anesthetic agent. In one embodiment, the composition comprises a macrophage proresolving polarizer (e.g. lipin-1), an absorbent agent, a blood vessel constricting agent (e.g., aluminum sulfate and/or caffeine), isolated fibrinogin and isolated thrombin (or isolated fibrin), a local anesthetic agent such as lidocaine, tetracaine, or benzocaine or prilocaine, and an antimicrobial antiseptic such as neomycin sulfate, polymyxin B, bacitracin zinc, or pramoxine, are employed. An exemplary formulation range is from 0.001:1 to 1:1 macrophage proresolving polarizer (e.g. lipin-1) to either absorbent agent to blood vessel constricting agent, with effective amounts of fibrin, a local anesthetic agent, and an antiseptic agent.

In one embodiment, to deliver the therapeutic agents, a powder may be used to treat wounds and, optionally, clot blood when applied directly to minor cut or scrapes. In one embodiment, a powder is available in spray form. In one embodiment, the powder is woven in fibers of pad in adhesive bandage or gauze. Other substances may also be woven into fibers of pad in adhesive bandages or gauze or fibers of paper like tissue to be used for blotting (added to facial tissue or smaller tissues similar to those used for facial oil absorbing). In one embodiment, a component may be adhered to a pad in adhesive bandage using bonding agent (e.g., glycerin). In one embodiment, the substance added to an antiseptic dispensing device or composition (gel, liquid, or spray). In one embodiment, a powder is compressed into stick form or into a mold that is packaged along with a disposable applicator. In one embodiment, the composition is available in a snap q-tip form (plastic tube in the middle, snapped releases substance to cotton tip for application).

To prepare compositions in accordance with the present invention, the individual components may be combined to result in the composition. The components may be combined using any method that does not negatively affect the functionality of each component and provides uniform or nearly uniform distribution of the components in the composition, such as but not limited to mixing under shear forces or under agitation. The components used in the composition are of pharmaceutically or medically acceptable purity.

In some embodiments, the macrophage proresolving polarizer (e.g. lipin-1) also includes one of, two of, or all three of IL-4, apoptotic cell (AC) derived lipids, ACs. In some embodiments the macrophage proresolving polarizer is a lipin-1 transcriptional coregulatory activity promoter. In some embodiments the lipin-1 transcriptional coregulatory activity promoter is in the form of RNA. In some embodiments the lipin-1 transcriptional coregulatory activity promoter is an inhibitor of lipin-1 macrophage pro-inflammatory responses enzymatic activity. In some embodiments, the lipin-1 macrophage pro-inflammatory responses enzymatic activity inhibitor is an antibody that inhibits. The amount of the macrophage proresolving polarizer used will depend on factors such as the size of the wound and severity, but can be from 0.001 g, 0.01 g, 0.1 g, 1.0 g, 5.0 g, and 10.0 g, for example.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A composition for wound healing, comprising:
an amount of a macrophage proresolving polarizer, wherein the amount is effective to promote wound healing.

2. The composition of claim 1, wherein the macrophage proresolving polarizer is lipin-1.

3. The composition of claim 2, wherein the composition includes one, two, or three of IL-4, apoptotic cells (ACs), and AC derived lipids.

4. The composition of claim 1, wherein the macrophage proresolving polarizer is a lipin-1 transcriptional coregulatory activity promoter.

5. The composition of claim 4, wherein the lipin-1 transcriptional coregulatory activity promoter is an inhibitor of lipin-1 macrophage pro-inflammatory responses enzymatic activity.

6. The composition of claim 5, wherein the lipin-1 macrophage pro-inflammatory response enzymatic activity inhibitor is an antibody.

7. The composition of claim 1, further comprising the composition being in one of powder form, aerosol form, and gel form.

8. The composition of claim 1, wherein the composition is applied to a support.

9. The composition of claim 1 further comprising an adsorbent.

10. The composition of claim 1, further comprising a blood clotting agent.

11. The composition of claim 1, further comprising a blood vessel constricting agent.

12. The composition of claim 1, further comprising a local anesthetic agent.

13. The composition of claim 1, further comprising an antimicrobial agent.

14. A method for promoting wound healing, comprising:
contacting a wound on a skin of a mammal with the composition of claim 1.

15. A kit comprising one or more containers including the composition of claim 1, in sterile packaging.

16. A wound healing device comprising:
a substrate; and
an amount of an amount of a macrophage proresolving polarizer, wherein the amount is effective to promote wound healing.

17. The wound healing device of claim 16, further comprising one, two, or three of a blood vessel constricting agent, an adsorbent, and a blood clotting agent.

18. The wound healing device of claim 16, wherein the macrophage proresolving polarizer is lipin-1.

19. The wound healing device of claim 18, wherein the composition includes one, two, three, four, or five of IL-4, apoptotic cells (ACs), AC derived lipids, Ly6C^{hi}, and Ly6C^{lo}.

20. The wound healing device of claim 16, wherein the macrophage proresolving polarizer is a lipin-1 transcriptional coregulatory activity promoter.
